(19) United States
(12) Patent Application Publication
(10) Pub. No.: US 2024/0327472 A1
(43) Pub. Date: Oct. 3, 2024

(54) MODIFIED CLOSTRIDIAL NEUROTOXINS

(71) Applicant: IPSEN BIOPHARM LIMITED, Wrexham (GB)

(72) Inventors: Kevin MOORE, Wrexham (GB); Andrew SPLEVINS, SR., Wrexham (GB); Teresa BARATA, Wrexham (GB); Daniel HIGAZI, Wrexham (GB); David SPENCER, Wrexham (GB)

(21) Appl. No.: 18/549,871

(22) PCT Filed: Mar. 11, 2022
(86) PCT No.: PCT/GB2022/050641

§ 371 Date: Sep. 8, 2023

(30) Foreign Application Priority Data
Mar. 11, 2021 (GB) 2103372.5

(51) Int. Cl. C07K 14/33; A61K 8/66; A61K 38/00; C12N 9/52

(52) U.S. Cl. CPC C07K 14/33; A61K 8/66; C12N 9/52; A61K 38/00; C12Y 304/24069

(57) ABSTRACT

The present invention is directed to a modified clostridial neurotoxin comprising a botulinum neurotoxin A (BoNT/A) H_{cc} domain, wherein the H_{cc} domain comprises a modification of methionine 1144 (MI 144), and wherein the modification increases oxidative resistance of the modified clostridial neurotoxin when compared to an otherwise identical clostridial neurotoxin lacking the modification. Also provided are (inter alia) corresponding methods for producing the same, methods for selecting oxidation resistant clostridial neurotoxins, nucleic acids encoding the same, and therapeutic uses of said modified clostridial neurotoxins.

### FIELD OF THE INVENTION

The present invention relates to modified clostridial neurotoxins, in particular, modified clostridial neurotoxins having increased oxidative resistance.

### BACKGROUND

Bacteria in the genus Clostridia produce highly potent and specific protein toxins, which can poison neurons and other cells to which they are delivered. Examples of such clostridial neurotoxins include the neurotoxins produced by C. tetani (TeNT) and by C. botulinum (BoNT) serotypes A-G, and X (see WO 2018/009903 A2), as well as those produced by C. baratii and C. butyricum.

The clostridial neurotoxins are among some of the most potent toxins known. For example, botulinum neurotoxins have median lethal dose (LD₅₀) values for mice ranging from 0.5 to 5 ng/kg, depending on the serotype. Both tetanus and botulinum neurotoxins act by inhibiting the function of affected neurons, specifically the release of neurotransmitters. While botulinum toxin acts at the neuromuscular junction and inhibits cholinergic transmission in the peripheral nervous system, tetanus toxin acts in the central nervous system.

In nature, clostridial neurotoxins are synthesised as a single-chain polypeptide that is modified post-translationally by a proteolytic cleavage event to form two polypeptide chains joined together by a disulphide bond. Cleavage occurs at a specific cleavage site, often referred to as the activation site, that is located between the cysteine residues that provide the inter-chain disulphide bond. It is this di-chain form that is the active form of the toxin. The two chains are termed the heavy chain (H-chain), which has a molecular mass of approximately 100 kDa, and the light chain (L-chain), which has a molecular mass of approximately 50 kDa. The H-chain comprises an N-terminal translocation component (H_{N} domain) and a C-terminal targeting component (Hc domain). The cleavage site is located between the L-chain and the translocation domain components. Following binding of the He domain to its target neuron and internalisation of the bound toxin into the cell via an endosome, the H_{N} domain translocates the L-chain across the endosomal membrane and into the cytosol, and the L-chain provides a protease function (also known as a non-cytotoxic protease).

Non-cytotoxic proteases act by proteolytically cleaving intracellular transport proteins known as SNARE proteins (e.g. SNAP-25, VAMP, or Syntaxin)—see Gerald K (2002) “Cell and Molecular Biology” (4th edition) John Wiley & Sons, Inc. The acronym SNARE derives from the term Soluble NSF Attachment Receptor, where NSF means N-ethylmaleimide-Sensitive Factor. SNARE proteins are integral to intracellular vesicle fusion, and thus to secretion of molecules via vesicle transport from a cell. The protease function is a zinc-dependent endopeptidase activity and exhibits a high substrate specificity for SNARE proteins. Accordingly, once delivered to a desired target cell, the non-cytotoxic protease is capable of inhibiting cellular secretion from the target cell. The L-chain proteases of clostridial neurotoxins are non-cytotoxic proteases that cleave SNARE proteins.

In view of the ubiquitous nature of SNARE proteins, clostridial neurotoxins such as botulinum neurotoxin have been successfully employed in a wide range of therapies.

For example, William J. Lipham, Cosmetic and Clinical Applications of Botulinum Toxin (Slack, Inc., 2004) describes the use of clostridial neurotoxins, such as botulinum neurotoxins (BoNTs), BoNT/A, BoNT/B, BoNT/C₁, BoNT/D, BoNT/E, BoNT/F and BoNT/G, and tetanus neurotoxin (TeNT), to inhibit neuronal transmission in a wide variety of therapeutic and cosmetic applications—as an example, BOTOX™ is currently approved as a therapeutic for the following indications: achalasia, adult spasticity, anal fissure, back pain, blepharospasm, bruxism, cervical dystonia, essential tremor, glabellar lines or hyperkinetic facial lines, headache, hemifacial spasm, hyperactivity of bladder, hyperhidrosis, juvenile cerebral palsy, multiple sclerosis, myoclonic disorders, nasal labial lines, spasmodic dysphonia, strabismus and VII nerve disorder. In addition, clostridial neurotoxin therapies are described for treating neuromuscular disorders (see U.S. Pat. No. 6,872,397); for treating uterine disorders (see US 2004/0175399); for treating ulcers and gastroesophageal reflux disease (see US 2004/0086531); for treating dystonia (see U.S. Pat. No. 6,319,505); for treating eye disorders (see US 2004/0234532); for treating blepharospasm (see US 2004/0151740); for treating strabismus (see US 2004/0126396); for treating pain (see U.S. Pat. Nos. 6,869,610, 6,641,820, 6,464,986, and 6,113,915); for treating fibromyalgia (see U.S. Pat. No. 6,623,742, US 2004/0062776); for treating lower back pain (see US 2004/0037852); for treating muscle injuries (see U.S. Pat. No. 6,423,319); for treating sinus headache (see U.S. Pat. No. 6,838,434); for treating tension headache (see U.S. Pat. No. 6,776,992); for treating headache (see U.S. Pat. No. 6,458,365); for reduction of migraine headache pain (see U.S. Pat. No. 5,714,469); for treating cardiovascular diseases (see U.S. Pat. No. 6,767,544); for treating neurological disorders such as Parkinson's disease (see U.S. Pat. Nos. 6,620,415, 6,306,403); for treating neuropsychiatric disorders (see US 2004/0180061, US 2003/0211121); for treating endocrine disorders (see U.S. Pat. No. 6,827,931); for treating thyroid disorders (see U.S. Pat. No. 6,740,321); for treating cholinergic influenced sweat gland disorders (see U.S. Pat. No. 6,683,049); for treating diabetes (see U.S. Pat. Nos. 6,337,075, 6,416,765); for treating a pancreatic disorder (see U.S. Pat. Nos. 6,261,572, 6,143,306); for treating cancers such as bone tumors (see U.S. Pat. Nos. 6,565,870, 6,368,605, 6,139,845, US 2005/0031648); for treating otic disorders (see U.S. Pat. Nos. 6,358,926, 6,265,379); for treating autonomic disorders such as gastrointestinal muscle disorders and other smooth muscle dysfunction (see U.S. Pat. No. 5,437,291); for treatment of skin lesions associated with cutaneous cell-proliferative disorders (see U.S. Pat. No. 5,670,484); for management of neurogenic inflammatory disorders (see U.S. Pat. No. 6,063,768); for reducing hair loss and stimulating hair growth (see U.S. Pat. No. 6,299,893); for treating downturned mouth (see U.S. Pat. No. 6,358,917); for reducing appetite (see US 2004/40253274); for dental therapies and procedures (see US 2004/0115139); for treating neuromuscular disorders and conditions (see US 2002/0010138); for treating various disorders and conditions and associated pain (see US 2004/0013692); for treating conditions resulting from mucus hypersecretion such as asthma and COPD (see WO 00/10598); and for treating non-neuronal conditions such as inflammation, endocrine conditions, exocrine conditions, immunological conditions, cardiovascular conditions, bone conditions (see WO 01/21213). All of the above publications are hereby incorporated by reference in their entirety.

The use of non-cytotoxic proteases such as clostridial neurotoxins (e.g. BoNTs and TeNT) in therapeutic and cosmetic treatments of humans and other mammals is anticipated to expand to an ever-widening range of diseases and ailments that can benefit from the properties of these toxins. In view of this, there is an increasing demand for large-scale manufacture of clostridial neurotoxins.

The large-scale manufacture of biotherapeutics, and clostridial neurotoxins in particular, is challenging, with the possibility for unwanted polypeptide modification and/or degradation at multiple stages of the process. One such unwanted modification is oxidation, which can occur during cellular expression of a clostridial neurotoxin, purification, bioprocessing, formulation, and/or storage. Indeed, oxidation is one of the principal degradation pathways for biotherapeutics. Oxidizing agents such as peroxides, dissolved oxygen, metal ions, light and free-radicals can catalyse oxidation of amino acids, such as methionine, cysteine, histidine, tryptophan, tyrosine, and phenylalanine (Torosantucci et al (2014), Pharm Res, 31, 541-553). In bioprocessing and formulation, metal catalysts may come from metal contaminated buffers and/or metal contact surfaces, with the metal-catalysed oxidation of histidine and methionine residues having been demonstrated to cause loss of activity, for example due to aggregation and/or precipitation of oxidized polypeptides. Moreover, oxidizing agents are typically employed for decontamination in large-scale manufacture. Clostridial neurotoxins are large polypeptides having many surface exposed amino acid residues that are candidates for oxidation.

For at least the reasons provided above, there is a need for a robust clostridial neurotoxin that is resistant to oxidation, thereby minimising unwanted modification and/or degradation during large-scale manufacture and/or storage. Moreover, such a clostridial neurotoxin could exhibit improved stabilisation at room temperature, e.g. as part of a liquid or lyophilised formulation. Advantageously, this would remove the need for maintenance of low temperatures during manufacture.

The present invention overcomes one or more of the above-mentioned problems and/or provides one or more of the advantages detailed above.

### SUMMARY OF THE INVENTION

The present inventors have found oxidation at methionine 1144 (M1144) of botulinum neurotoxin A (BoNT/A) to be a principal cause of oxidation-dependent activity loss. Advantageously, by modifying M1144 to prevent its oxidation (e.g. via substitution with an oxidation resistant amino acid residue and/or deletion of M1144), the inventors have found that oxidation-dependent activity loss can be minimised/avoided. Surprisingly, modification of M1144 not only minimises/avoids oxidation-dependent activity loss but may, in fact, increase activity of the resultant modified neurotoxin.

M1144 is an amino acid present in the C-terminal portion of the He domain of BoNT/A (Hec domain) and is involved in binding of BoNT/A to synaptic vesicle glycoprotein (SV2) on target neuronal cells. Given that this residue is involved in SV2 binding, it is believed that any clostridial neurotoxin comprising a BoNT/A H_{cc} domain suitably modified at M1144 will exhibit the improved properties described herein.

### DETAILED DESCRIPTION

In one aspect, the invention provides a modified clostridial neurotoxin comprising a botulinum neurotoxin A (BoNT/A) H_{cc} domain, wherein the H_{cc} domain comprises a modification of methionine 1144 (M1144), and wherein the modification increases oxidative resistance of the modified clostridial neurotoxin when compared to an otherwise identical clostridial neurotoxin lacking the modification.

The term “modified clostridial neurotoxin” as used herein refers to a clostridial neurotoxin that does not exist in nature and that comprises a modification of methionine 1144 (M1144), wherein the modification increases oxidative resistance of the modified clostridial neurotoxin when compared to an otherwise identical clostridial neurotoxin lacking the modification. Thus, the term “modified clostridial neurotoxin” excludes a natural variant clostridial neurotoxin that comprises botulinum neurotoxin A (BoNT/A) H_{cc} domain, wherein the H_{cc} domain comprises an amino acid that is resistant to oxidation at amino acid 1144.

A modified clostridial neurotoxin may be a single-chain modified clostridial neurotoxin or a di-chain modified clostridial toxin. Preferably, a modified clostridial neurotoxin is a di-chain modified clostridial toxin comprising a light chain and a heavy chain joined together by a disulphide bond.

A modified clostridial neurotoxin of the invention binds to a clostridial neurotoxin target cell, specifically at least a BoNT/A target cell. Said modified clostridial neurotoxin binds to SV2 (e.g. SV2c). In one embodiment, a modified clostridial neurotoxin of the invention binds to SV2 with a higher affinity than an otherwise identical clostridial neurotoxin lacking a claimed modification. In particular, as shown herein, a modified clostridial neurotoxin comprising a M1144V or M1144L substitution binds to SV2 with a higher affinity than an otherwise identical clostridial neurotoxin lacking a claimed modification. A higher affinity may be expressed as a lower K_{D} value (preferably a statistically-significantly lower value) when compared to that of an otherwise identical clostridial neurotoxin lacking a claimed modification. A K_{D} value is preferably determined using an assay described in Example 6 herein.

A modified clostridial neurotoxin of the invention preferably exhibits improved activity (e.g. statistically-significantly improved activity) when compared with an otherwise identical clostridial neurotoxin lacking a claimed modification.

The amino acid position numbering referred to herein is defined by alignment with SEQ ID NO: 2. For example, a position presented as 1144 may not be amino acid number 1144 of a given polypeptide, but instead is the methionine residue that corresponds to M1144 of SEQ ID NO: 2 when said polypeptide is aligned with SEQ ID NO: 2. SEQ ID NO: 2 is an unmodified BoNT/A1 polypeptide sequence that exists in nature. As an example, M1140 of BoNT/A3 corresponds to M1144 of BoNT/A1. Thus, modification of M1144 as used herein in reference to BoNT/A3 means modification of position M1144 when aligned with SEQ ID NO: 2 (i.e. modification of BoNT/A3 residue M1140). Alignment may be carried out using any of the methods described herein for determining sequence homology and/or % sequence identity.

An “otherwise identical clostridial neurotoxin” that lacks a modification of the invention may be an unmodified clostridial neurotoxin described herein and/or a clostridial neurotoxin comprising an unmodified BoNT/A H_{cc} domain described herein. Said “otherwise identical clostridial neurotoxin” may be one that exists in nature.

Any modification that increases oxidative resistance of the modified clostridial neurotoxin may be employed in the present invention. A modification may be a substitution of M1144, a deletion of M1144 or an indel at a site comprising (preferably consisting of) M1144. Preferably, a modification is substitution of M1144 with an amino acid that is resistant to oxidation.

The term “deletion” as used herein refers to removal of one or more amino acid residues of a polypeptide without replacement of one or more amino acid residues at the site of deletion. Thus, where one amino acid residue has been deleted from a polypeptide sequence having x number of amino acid residues (for example), the resultant polypeptide has x−1 amino acid residues.

The term “indel” as used herein refers to deletion of one or more amino acid residues of a polypeptide and insertion at the deletion site of a different number of amino acid residues (either greater or fewer amino acid residues) when compared to the number of amino acid residues deleted. Thus, for an indel where two amino acid residues have been deleted from a polypeptide sequence having x number of amino acid residues (for example), the resultant polypeptide has x−1 amino acid residues or x+≥1 amino acid residues. The insertion and deletion can be carried out in any order, sequentially or simultaneously.

The term “substitution” as used herein refers to replacement of one or more amino acid residues with the same number of amino acid residues at the same site. Thus, for a substitution of a polypeptide sequence having x number of amino acid residues (for example), the resultant polypeptide also has x amino acid residues. Preferably a substitution is a substitution at a single amino acid position.

The term “insertion” as used herein refers to addition of one or more amino acid residues of a polypeptide without deletion of one or more amino acid residues of the polypeptide at the site of insertion. Thus, where one amino acid residue has been inserted into a polypeptide sequence having x number of amino acid residues (for example), the resultant polypeptide has x+1 amino acid residues.

As discussed in more detail later, typically a modification is carried out by either modifying a nucleic acid encoding a native clostridial neurotoxin such that the modified clostridial neurotoxin encoded by the nucleic acid comprises the modification(s). Alternatively, a nucleic acid that encodes a modified clostridial neurotoxin comprising the modification(s) can be synthesized.

An “amino acid that is resistant to oxidation” as used herein preferably means any amino acid that is more resistant to oxidation than methionine. An amino acid that is resistant to oxidation may be selected from: valine, leucine, glycine, threonine, alanine, isoleucine, aspartic acid, glutamic acid, arginine, lysine, asparagine, glutamine, serine, and proline. In one embodiment an amino acid that is resistant to oxidation may be selected from: valine, leucine, and glycine. Preferably, an amino acid that is resistant to oxidation is selected from valine and leucine. In one embodiment, an amino acid that is resistant to oxidation is not glycine.

Alternatively, an amino acid that is resistant to oxidation may be a non-natural or non-standard amino acid, such as norleucine, isovaline, α-methyl valine, cycloleucine, or allo-threonine.

A modified clostridial neurotoxin of the invention may be at least 1%, 5%, 10%, 20%, 50%, 75%, 90% or 100% more resistant to oxidation than an otherwise identical clostridial neurotoxin lacking the modification.

Oxidation (including resistance to oxidation) of a clostridial neurotoxin may be assessed using a forced oxidation assay. Preferably, oxidation (e.g. resistance to oxidation) of a clostridial neurotoxin is assessed using a forced oxidation assay described in the present Examples (see “Forced Oxidation Study”).

In one embodiment, a modified clostridial neurotoxin of the invention may be substantially resistant to oxidation at a position corresponding to M1144 of SEQ ID NO: 2 when exposed to forced oxidation conditions. The term “substantially resistant to oxidation” as used in this context may mean that less than 10%, 5%, or 1% (preferably less than 0.1%) of modified clostridial neurotoxins in a composition are oxidised at a position corresponding to M1144 of SEQ ID NO: 2 under forced oxidation conditions. In other words, preferably, a modified clostridial neurotoxin of the invention is incapable of being oxidised at a position corresponding to M1144 of SEQ ID NO: 2 under forced oxidation conditions. In this context, the position corresponding to M1144 is the modified position.

A modified clostridial neurotoxin may comprise one or more further modifications of one or more surface exposed amino acid residues, e.g. one or more surface exposed amino acid residues susceptible to oxidation. Thus, a modified clostridial neurotoxin may comprise one or more further modifications of the following amino acids, which are susceptible to oxidation: methionine, cysteine, histidine, tryptophan, tyrosine, and phenylalanine (preferably methionine). For example, a modified clostridial neurotoxin may comprise one or more further modifications at an amino acid present in the H_{cc} domain (preferably in the SV2 binding domain), e.g. when compared to an unmodified H_{cc} domain (preferably an unmodified SV2 binding domain). Preferably, however, the modified clostridial neurotoxin does not comprise one or more further modifications in the H_{cc} domain, e.g. when compared to an unmodified H_{cc} domain, more preferably does not comprise one or more further modifications in the SV2 binding domain, e.g. when compared to an unmodified SV2 binding domain. In some embodiments the modified clostridial neurotoxin does not comprise one or more further modifications in the He domain, e.g. when compared to an unmodified He domain. In one embodiment the modified clostridial neurotoxin does not comprise one or more further modifications in the heavy chain, e.g. when compared to an unmodified heavy chain. In one embodiment the modified clostridial neurotoxin does not comprise one or more further modifications in the light chain, e.g. when compared to an unmodified light chain. In another embodiment the modified clostridial neurotoxin does not comprise one or more further modifications in the light chain e.g. when compared to an unmodified light chain, and does not comprise one or more further modifications in the heavy chain, e.g. when compared to an unmodified heavy chain.

In one aspect the invention provides a modified clostridial neurotoxin comprising a modified BoNT/A1 H_{cc} domain, a modified BoNT/A3 H_{cc} domain or a modified BoNT/A4 H_{cc} domain comprising RX₁X₂VX₃TTNIYLNSX₄LYX₅GT (SEQ ID NO: 102), wherein: X, is D or G; X₂ is S or N; X₃ is an amino acid that is resistant to oxidation; X₄ is S or T; and X₅ is M or R. Preferably X₄ is S. In said modified clostridial neurotoxin M1144 has been substituted with an amino acid that is resistant to oxidation.

SEQ ID NO: 102 is a consensus sequence comprised in the BoNT/A1, BoNT/A3, and BoNT/A4 SV2c binding domain of the H_{cc} domain, but wherein X₃ is an amino acid that is resistant to oxidation. In the native sequence, X₃ is methionine as shown in the table below:

| SEQ ID NO: | Sequence | Strain |
|---|---|---|
| 103 (part of SEQ | RGSVMTTNIYLNSSLYRGT | A1 Hall Str |
| ID NO: 2) | | (Reference) |
| 104 | RGNVMTTNIYLNSSLYMGT | A1 CDC297 |
| 105 | RGSVMTTNIYLNSTLYMGT | A3 Loch |
| (part of SEQ ID | | Maree |
| NO: 36) | | |
| 106 (part of SEQ | RDNVMTTNIYLNSSLYMGT | A4 |
| ID NO: 54) | | |

Any modified clostridial neurotoxin described herein may comprise a modified H_{cc} domain comprising (preferably consisting of) RX₁X₂VX₃TTNIYLNSX₄LYX₅GT (SEQ ID NO: 102), wherein: X, is D or G; X₂ is S or N; X₃ is an amino acid that is resistant to oxidation; X₄ is S or T; and X₅ is M or R. Preferably X₄ is S.

A modified clostridial neurotoxin described herein may comprise a modified H_{cc} domain comprising (preferably consisting of): RGSVXTTNIYLNSSLYRGT (SEQ ID NO: 107), RGNVXTTNIYLNSSLYMGT (SEQ ID NO: 108), RGSVXTTNIYLNSTLYMGT (SEQ ID NO: 109) or RDNVXTTNIYLNSSLYMGT (SEQ ID NO: 110), wherein X is an amino acid that is resistant to oxidation.

In one aspect the invention provides a modified clostridial neurotoxin comprising a modified BoNT/A1 H_{cc} domain, a modified BoNT/A3 H_{cc} domain or a modified BoNT/A4 H_{cc} domain comprising RX₁X₂VTTNIYLNSX₃LYX₄GT (SEQ ID NO: 111), wherein: X, is D or G; X₂ is S or N; X₃ is S or T; and X₄ is M or R. Preferably X₃ is S. In said modified clostridial neurotoxin M1144 has been deleted.

Any modified clostridial neurotoxin described herein may comprise a modified H_{cc} domain comprising (preferably consisting of) RX₁X₂VTTNIYLNSX₃LYX₄GT (SEQ ID NO: 111), wherein: X, is D or G; X₂ is S or N; X₃ is S or T; and X₄ is M or R. Preferably X₃ is S.

A modified clostridial neurotoxin described herein may comprise a modified H_{cc} domain comprising (preferably consisting of): RGSVTTNIYLNSSLYRGT (SEQ ID NO: 112), RGNVTTNIYLNSSLYMGT (SEQ ID NO: 113), RGSVTTNIYLNSTLYMGT (SEQ ID NO: 114) or RDNVTTNIYLNSSLYMGT (SEQ ID NO: 115).

In one embodiment, a modified clostridial neurotoxin of the invention does not comprise a BoNT/A2 H_{cc} domain. In one embodiment, a modified clostridial neurotoxin of the invention does not comprise a BoNT/A5 H_{cc} domain. In one embodiment, a modified clostridial neurotoxin of the invention does not comprise a BoNT/A6 H_{cc} domain. In one embodiment, a modified clostridial neurotoxin of the invention does not comprise a BoNT/A7 H_{cc} domain. In one embodiment, a modified clostridial neurotoxin of the invention does not comprise a BoNT/A8 H_{cc} domain. In one embodiment, a modified clostridial neurotoxin of the invention does not comprise a BoNT/A2 H_{cc} domain, a BoNT/A5 H_{cc} domain, a BoNT/A6 H_{cc} domain, a BoNT/A7 H_{cc} domain, or a BoNT/A8 H_{cc} domain.

Preferably, a modified clostridial neurotoxin of the invention is not a BoNT/A2, a BoNT/A5, a BoNT/A6, a BoNT/A7, or a BoNT/A8. A BoNT/A2 may comprise a polypeptide sequence having at least 70% sequence identity to SEQ ID NO: 139. For example, a BoNT/A2 may comprise a polypeptide sequence having at least 80%, 90%, 95%, or 98% sequence identity to SEQ ID NO: 139. Preferably BoNT/A2 comprises (or consists of) SEQ ID NO: 139. A BoNT/A5 may comprise a polypeptide sequence having at least 70% sequence identity to SEQ ID NO: 140 or 141. For example, a BoNT/A5 may comprise a polypeptide sequence having at least 80%, 90%, 95%, or 98% sequence identity to SEQ ID NO: 140 or 141. Preferably BoNT/A5 comprises (or consists of) SEQ ID NO: 140 or 141. A BoNT/A6 may comprise a polypeptide sequence having at least 70% sequence identity to SEQ ID NO: 142. For example, a BoNT/A6 may comprise a polypeptide sequence having at least 80%, 90%, 95%, or 98% sequence identity to SEQ ID NO: 142. Preferably BoNT/A6 comprises (or consists of) SEQ ID NO: 142. A BoNT/A7 may comprise a polypeptide sequence having at least 70% sequence identity to SEQ ID NO: 143. For example, a BoNT/A7 may comprise a polypeptide sequence having at least 80%, 90%, 95%, or 98% sequence identity to SEQ ID NO: 143. Preferably BoNT/A7 comprises (or consists of) SEQ ID NO: 143. A BoNT/A8 may comprise a polypeptide sequence having at least 70% sequence identity to SEQ ID NO: 144. For example, a BoNT/A8 may comprise a polypeptide sequence having at least 80%, 90%, 95%, or 98% sequence identity to SEQ ID NO: 144. Preferably BoNT/A8 comprises (or consists of) SEQ ID NO: 144.

Preferably, a modified clostridial neurotoxin of the invention does not comprise an H_{cc} domain corresponding to (or of): UniProtKB Accession No. D3|V23 (Sequence Version 1); UniProtKB Accession No. C7BEA8 (Sequence Version 1); or UniProtKB Accession No. C11PK2 (Sequence Version 1). Most preferably, a modified clostridial neurotoxin of the invention does not comprise a polypeptide sequence corresponding to (or of): UniProtKB Accession No. D3IV23 (Sequence Version 1); UniProtKB Accession No. C7BEA8 (Sequence Version 1); or UniProtKB Accession No. C11PK2 (Sequence Version 1).

A modified clostridial neurotoxin of the invention may comprise a modified BoNT/A1 H_{cc} domain, a modified BoNT/A3 H_{cc} domain or a modified BoNT/A4 H_{cc} domain, preferably a modified BoNT/A1 H_{cc} domain.

In one embodiment, a modified clostridial neurotoxin of the invention may comprise a modification at M1144 of an unmodified BoNT/A1 H_{cc} domain. An unmodified BoNT/A1 H_{cc} domain may comprise a polypeptide sequence having at least 70% sequence identity to any one of SEQ ID NOs: 62, 70 or 78. In one embodiment, an unmodified BoNT/A1 H_{cc} domain may comprise a polypeptide sequence having at least 80%, 90%, 95%, or 98% sequence identity to any one of SEQ ID NOs: 62, 70 or 78. Preferably, an unmodified BoNT/A1 H_{cc} domain comprises (more preferably consists of) any one of SEQ ID NOs: 62, 70 or 78. Of the sequences indicated, SEQ ID NO: 70 is most preferred.

A modified BoNT/A1 H_{cc} domain may comprise a polypeptide sequence having at least 70% sequence identity to any one of SEQ ID NOs: 63-69, 71-77 or 79-85. In one embodiment, a modified BoNT/A1 H_{cc} domain may comprise a polypeptide sequence having at least 80%, 90%, 95%, or 98% sequence identity to any one of SEQ ID NOs: 63-69, 71-77 or 79-85. Preferably, a modified BoNT/A1 H_{cc} domain comprises (more preferably consists of) any one of SEQ ID NOs: 63-69, 71-77 or 79-85. Of the sequences indicated, SEQ ID NOs: 71-77 are most preferred.

In one embodiment, a modified clostridial neurotoxin of the invention may comprise a modification at M1144 of an unmodified BoNT/A3 H_{cc} domain. An unmodified BoNT/A3 H_{cc} domain may comprise a polypeptide sequence having at least 70% sequence identity to SEQ ID NO: 86. In one embodiment, an unmodified BoNT/A3 H_{cc} domain may comprise a polypeptide sequence having at least 80%, 90%, 95%, or 98% sequence identity to SEQ ID NO: 86. Preferably, an unmodified BoNT/A3 H_{cc} domain comprises (more preferably consists of) SEQ ID NO: 86.

A modified BoNT/A3 H_{cc} domain may comprise a polypeptide sequence having at least 70% sequence identity to any one of SEQ ID NOs: 87-93. In one embodiment, a modified BoNT/A3 H_{cc} domain may comprise a polypeptide sequence having at least 80%, 90%, 95%, or 98% sequence identity to any one of SEQ ID NOs: 87-93. Preferably, a modified BoNT/A3 H_{cc} domain comprises (more preferably consists of) any one of SEQ ID NOs: 87-93.

In one embodiment, a modified clostridial neurotoxin of the invention may comprise a modification at M1144 of an unmodified BoNT/A4 H_{cc} domain. An unmodified BoNT/A4 H_{cc} domain may comprise a polypeptide sequence having at least 70% sequence identity to SEQ ID NO: 94. In one embodiment, an unmodified BoNT/A4 H_{cc} domain may comprise a polypeptide sequence having at least 80%, 90%, 95%, or 98% sequence identity to SEQ ID NO: 94. Preferably, an unmodified BoNT/A4 H_{cc} domain comprises (more preferably consists of) SEQ ID NO: 94.

A modified BoNT/A4 H_{cc} domain may comprise a polypeptide sequence having at least 70% sequence identity to any one of SEQ ID NOs: 95-101. In one embodiment, a modified BoNT/A4 H_{cc} domain may comprise a polypeptide sequence having at least 80%, 90%, 95%, or 98% sequence identity to any one of SEQ ID NOs: 95-101. Preferably, a modified BoNT/A4 H_{cc} domain comprises (more preferably consists of) any one of SEQ ID NOs: 95-101.

A modified clostridial neurotoxin of the invention may be a modified BoNT/A1, a modified BoNT/A3 or a modified BoNT/A4. Preferably, a modified clostridial neurotoxin of the invention is a modified BoNT/A1.

In one embodiment, a modified clostridial neurotoxin of the invention may comprise a modification at M1144 of an unmodified BoNT/A1. An unmodified BoNT/A1 may comprise a polypeptide sequence having at least 70% sequence identity to any one of SEQ ID NOs: 2, 11, 20, 29 or 38. In one embodiment, an unmodified BoNT/A1 may comprise a polypeptide sequence having at least 80%, 90%, 95%, or 98% sequence identity to any one of SEQ ID NOs: 2, 11, 20, 29 or 38. Preferably, an unmodified BoNT/A1 comprises (more preferably consists of) any one of SEQ ID NOs: 2, 11, 20, 29 or 38. Of the sequences indicated, SEQ ID NO: 11 is most preferred.

A modified BoNT/A1 may comprise a polypeptide sequence having at least 70% sequence identity to any one of SEQ ID NOs: 3-9, 12-18, 21-27, 30-36 or 39-45. In one embodiment, a modified BoNT/A1 may comprise a polypeptide sequence having at least 80%, 90%, 95%, or 98% sequence identity to any one of SEQ ID NOs: 3-9, 12-18, 21-27, 30-36 or 39-45. Preferably, a modified BoNT/A1 comprises (more preferably consists of) any one of SEQ ID NOs: 3-9, 12-18, 21-27, 30-36 or 39-45. Of the sequences indicated, SEQ ID NOs: 12-18 are most preferred.

In one embodiment, a modified clostridial neurotoxin of the invention may comprise a modification at M1144 of an unmodified BoNT/A3. An unmodified BoNT/A3 may comprise a polypeptide sequence having at least 70% sequence identity to SEQ ID NO: 46. In one embodiment, an unmodified BoNT/A3 may comprise a polypeptide sequence having at least 80%, 90%, 95%, or 98% sequence identity to SEQ ID NO: 46. Preferably, an unmodified BoNT/A3 comprises (more preferably consists of) SEQ ID NO: 46.

A modified BoNT/A3 may comprise a polypeptide sequence having at least 70% sequence identity to any one of SEQ ID NOs: 47-53. In one embodiment, a modified BoNT/A3 may comprise a polypeptide sequence having at least 80%, 90%, 95%, or 98% sequence identity to any one of SEQ ID NOs: 47-53. Preferably, a modified BoNT/A3 comprises (more preferably consists of) any one of SEQ ID NOs: 47-53.

In one embodiment, a modified clostridial neurotoxin of the invention may comprise a modification at M1144 of an unmodified BoNT/A4. An unmodified BoNT/A4 may comprise a polypeptide sequence having at least 70% sequence identity to SEQ ID NO: 54. In one embodiment, an unmodified BoNT/A4 may comprise a polypeptide sequence having at least 80%, 90%, 95%, or 98% sequence identity to SEQ ID NO: 54. Preferably, an unmodified BoNT/A4 comprises (more preferably consists of) SEQ ID NO: 54.

A modified BoNT/A4 may comprise a polypeptide sequence having at least 70% sequence identity to any one of SEQ ID NOs: 55-61. In one embodiment, a modified BoNT/A4 may comprise a polypeptide sequence having at least 80%, 90%, 95%, or 98% sequence identity to any one of SEQ ID NOs: 55-61. Preferably, a modified BoNT/A4 comprises (more preferably consists of) any one of SEQ ID NOs: 55-61.

The unmodified BoNT/A1 H_{cc} domains having SEQ ID NOs: 70 and 78 and corresponding BoNT/A1 polypeptides having SEQ ID NOs: 11, 20, 29, and 38 are themselves modified with respect to SEQ ID NOs: 2 and 62 (which exist in nature). Said clostridial neurotoxins are taught in WO 2015/004461 A1, which is incorporated herein by reference in its entirety. The modifications present in said clostridial neurotoxins provides increased potency and/or duration of action thereby allowing reduced dosages of the neurotoxins to be used compared to known clostridial neurotoxin therapeutics (or increased dosages without any additional adverse effects), thus providing further advantages. Indeed, said neurotoxins demonstrate a reduction in, or absence of, side effects compared to the use of an equivalent clostridial neurotoxin lacking said one or more amino acid modifications. This is achieved via modifications of surface exposed amino acid residues that increase the isoelectric point of the modified neurotoxins. Without wishing to be bound by theory, it is believed that said neurotoxins display longer tissue retention times at the site of administration due to favourable electrostatic interactions between the modified clostridial neurotoxin and anionic extracellular components (such as cell membranes and heparin sulphate proteoglycans) at the site of administration.

For the purpose of the present invention, SEQ ID NOs: 11, 20, 29, 38, 70, and 78 are categorised as “unmodified” as they comprise methionine at position 1144.

WO 2015/004461 A1 teaches that suitable modification of one or more of ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277 increases the isoelectric point of the modified clostridial neurotoxin to provide the above-mentioned advantages.

Thus, a modified clostridial neurotoxin may comprise a modified BoNT/A H_{cc} domain (preferably BoNT/A1 H_{cc} domain) comprising a further modification at one or more amino acid residue(s) selected from: ASN 1188, ASP 1213, GLY 1215, ASN 1216, GLN 1229, ASN 1242, ASN 1243, SER 1274, and THR 1277.

Thus, a modified clostridial neurotoxin (preferably modified BoNT/A1) may comprise a further modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277.

The modification may be a modification when compared to SEQ ID NO: 2, wherein the amino acid residue numbering is determined by alignment with SEQ ID NO: 2.

The further amino acid residue(s) indicated for modification are surface exposed amino acid residue(s).

It is preferred that a modified clostridial neurotoxin comprises a further modification at one or more amino acid residue(s) selected from: ASN 886, ASN 930, ASN 954, SER 955, GLN 991, ASN 1025, ASN 1026, ASN 1052, ASN 1188, ASP 1213, GLY 1215, ASN 1216, GLN 1229, ASN 1242, ASN 1243, SER 1274 and THR 1277.

In a particularly preferred embodiment, a modified clostridial neurotoxin of the invention comprises only a modification at M1144 and a further modification at one or more of: ASN 886, ASN 930, ASN 954, SER 955, GLN 991, ASN 1025, ASN 1026, ASN 1052, ASN 1188, ASP 1213, GLY 1215, ASN 1216, GLN 1229, ASN 1242, ASN 1243, SER 1274 and THR 1277. For example, in one embodiment, a modified clostridial neurotoxin of the invention comprises SEQ ID NO: 2 modified only at M1144 and a further modification at one or more of: ASN 886, ASN 930, ASN 954, SER 955, GLN 991, ASN 1025, ASN 1026, ASN 1052, ASN 1188, ASP 1213, GLY 1215, ASN 1216, GLN 1229, ASN 1242, ASN 1243, SER 1274 and THR 1277. Put another way, it is preferred that the modifications when compared to SEQ ID NO: 2 consist of modifications at M1144 and a further modification at one or more of: ASN 886, ASN 930, ASN 954, SER 955, GLN 991, ASN 1025, ASN 1026, ASN 1052, ASN 1188, ASP 1213, GLY 1215, ASN 1216, GLN 1229, ASN 1242, ASN 1243, SER 1274 and THR 1277.

A modified clostridial neurotoxin may comprise at least 2, 3, 4, 5, 6 or 7 (preferably 7) further modifications at the indicated amino acid residue(s) in addition to a modification at M1144. A modified clostridial neurotoxin may comprise (preferably consist of) 1-30, 3-20, or 5-10 further amino acid modifications.

The further modification may be selected from:
i. substitution of an acidic surface exposed amino acid residue with a basic amino acid residue;
ii. substitution of an acidic surface exposed amino acid residue with an uncharged amino acid residue;
iii. substitution of an uncharged surface exposed amino acid residue with a basic amino acid residue;
iv. insertion of a basic amino acid residue; and
v. deletion of an acidic surface exposed amino acid residue.

A further modification as indicated above results in a modified clostridial neurotoxin that has an increased positive surface charge and increased isoelectric point when compared to the corresponding unmodified clostridial neurotoxin.

The isoelectric point (pI) is a specific property of a given protein. As is well known in the art, proteins are made from a specific sequence of amino acids (also referred to when in a protein as amino acid residues). Each amino acid of the standard set of twenty has a different side chain (or R group), meaning that each amino acid residue in a protein displays different chemical properties such as charge and hydrophobicity. These properties may be influenced by the surrounding chemical environment, such as the temperature and pH. The overall chemical characteristics of a protein will depend on the sum of these various factors.

Certain amino acid residues (detailed below) possess ionisable side chains that may display an electric charge depending on the surrounding pH. Whether such a side chain is charged or not at a given pH depends on the pKa of the relevant ionisable moiety, wherein pKa is the negative logarithm of the acid dissociation constant (Ka) for a specified proton from a conjugate base.

For example, acidic residues such as aspartic acid and glutamic acid have side chain carboxylic acid groups with pKa values of approximately 4.1 (precise pKa values may depend on temperature, ionic strength and the microenvironment of the ionisable group). Thus, these side chains exhibit a negative charge at a pH of 7.4 (often referred to as “physiological pH”). At low pH values, these side chains will become protonated and lose their charge.

Conversely, basic residues such as lysine and arginine have nitrogen-containing side chain groups with pKa values of approximately 10-12. These side chains therefore exhibit a positive charge at a pH of 7.4. These side chains will become de-protonated and lose their charge at high pH values.

The overall (net) charge of a protein molecule therefore depends on the number of acidic and basic residues present in the protein (and their degree of surface exposure) and on the surrounding pH. Changing the surrounding pH changes the overall charge on the protein.

Accordingly, for every protein there is a given pH at which the number of positive and negative charges is equal and the protein displays no overall net charge. This point is known as the isoelectric point (pI). The isoelectric point is a standard concept in protein biochemistry with which the skilled person would be familiar.

The isoelectric point (pI) is therefore defined as the pH value at which a protein displays a net charge of zero. An increase in pI means that a higher pH value is required for the protein to display a net charge of zero. Thus, an increase in pI represents an increase in the net positive charge of a protein at a given pH. Conversely, a decrease in pI means that a lower pH value is required for the protein to display a net charge of zero. Thus, a decrease in pI represents a decrease in the net positive charge of a protein at a given pH.

Methods of determining the pI of a protein are known in the art and would be familiar to a skilled person. By way of example, the pI of a protein can be calculated from the average pKa values of each amino acid present in the protein (“calculated pI”). Such calculations can be performed using computer programs known in the art, such as the Compute pI/MW Tool from ExPASy (https://web.expasy.org/compute_pi/), which is the preferred method for calculating pI in accordance with the present invention. Comparisons of pI values between different molecules should be made using the same calculation technique/program.

Where appropriate, the calculated pI of a protein can be confirmed experimentally using the technique of isoelectric focusing (“observed pI”). This technique uses electrophoresis to separate proteins according to their pI. Isoelectric focusing is typically performed using a gel that has an immobilised pH gradient. When an electric field is applied, the protein migrates through the pH gradient until it reaches the pH at which it has zero net charge, this point being the pI of the protein. Results provided by isoelectric focusing are typically relatively low-resolution in nature, and thus the present inventors believe that results provided by calculated pI (as described above) are more appropriate to use.

Throughout the present specification, “pI” means “calculated pI” unless otherwise stated.

The pI of a protein may be increased or decreased by altering the number of basic and/or acidic groups displayed on its surface. This can be achieved by modifying one or more amino acids of the protein. For example, an increase in pI may be provided by reducing the number of acidic residues, or by increasing the number of basic residues.

A modified clostridial neurotoxin comprising a further modification may have a pI value that is at least 0.2, 0.4, 0.5 or 1 pI units higher than that of an unmodified clostridial neurotoxin (e.g. SEQ ID NO: 2). Preferably, a modified clostridial neurotoxin may have a pI of at least 6.6, e.g. at least 6.8.

The properties of the 20 standard amino acids are indicated in the table below:

| Amino Acid | | | Side Chain |
|---|---|---|---|
| Aspartic acid | Asp | D | Charged (acidic) |
| Glutamic acid | Glu | E | Charged (acidic) |
| Arginine | Arg | R | Charged (basic) |
| Lysine | Lys | K | Charged (basic) |
| Histidine | His | H | Uncharged (polar) |
| Asparagine | Asn | N | Uncharged (polar) |
| Glutamine | Gln | Q | Uncharged (polar) |
| Serine | Ser | S | Uncharged (polar) |
| Threonine | Thr | T | Uncharged (polar) |
| Tyrosine | Tyr | Y | Uncharged (polar) |
| Methionine | Met | M | Uncharged (polar) |
| Tryptophan | Trp | W | Uncharged (polar) |
| Cysteine | Cys | C | Uncharged (polar) |
| Alanine | Ala | A | Uncharged (hydrophobic) |
| Glycine | Gly | G | Uncharged (hydrophobic) |
| Valine | Val | V | Uncharged (hydrophobic) |
| Leucine | Leu | L | Uncharged (hydrophobic) |
| Isoleucine | Ile | — | Uncharged (hydrophobic) |
| Proline | Pro | P | Uncharged (hydrophobic) |
| Phenylalanine | Phe | F | Uncharged (hydrophobic) |

The following amino acids are considered charged amino acids: aspartic acid (negative), glutamic acid (negative), arginine (positive), and lysine (positive).

At a pH of 7.4, the side chains of aspartic acid (pKa 3.1) and glutamic acid (pKa 4.1) have a negative charge, while the side chains of arginine (pKa 12.5) and lysine (pKa 10.8) have a positive charge. Aspartic acid and glutamic acid are referred to as acidic amino acid residues. Arginine and lysine are referred to as basic amino acid residues.

The following amino acids are considered uncharged, polar (meaning they can participate in hydrogen bonding) amino acids: asparagine, glutamine, histidine, serine, threonine, tyrosine, cysteine, methionine, and tryptophan.

The following amino acids are considered uncharged, hydrophobic amino acids: alanine, valine, leucine, isoleucine, phenylalanine, proline, and glycine.

Preferably, the further modification is a substitution. The replacement amino acid residue may be one of the 20 standard amino acids, as described above. Alternatively, the replacement amino acid in an amino acid substitution may be a non-standard or non-natural amino acid (an amino acid that is not part of the standard set of 20 described above). By way of example, the replacement amino acid may be a basic non-standard or non-natural amino acid, e.g. L-Ornithine, L-2-amino-3-guanidinopropionic acid, or D-isomers of Lysine, Arginine and Ornithine). Methods for introducing non-standard or non-natural amino acids into proteins are known in the art and include recombinant protein synthesis using E. coli auxotrophic expression hosts. In one embodiment, the substitution is selected from: substitution of an acidic amino acid residue with a basic amino acid residue, substitution of an acidic amino acid residue with an uncharged amino acid residue, and substitution of an uncharged amino acid residue with a basic amino acid residue. In one embodiment, wherein the substitution is a substitution of an acidic amino acid residue with an uncharged amino acid residue, the acidic amino acid residue is replaced with its corresponding uncharged amide amino acid residue (i.e. aspartic acid is replaced with asparagine, and glutamic acid is replaced with glutamine).

Preferably, the basic amino acid residue is a lysine residue or an arginine residue. In other words, the substitution is preferably substitution with lysine or arginine. Most preferably, the modification is substitution with lysine.

Preferably, a modified clostridial neurotoxin comprising the one or more further modifications comprises between 4 and 40 amino acid modifications located in the clostridial toxin H_{CN} domain. Said modified clostridial neurotoxin preferably also has pI of at least 6.6. In addition to a modification at M1144, said modified clostridial neurotoxin preferably comprises modifications of at least 4 amino acids selected from: ASN 886, ASN 930, ASN 954, SER 955, GLN 991, ASN 1025, ASN 1026, and ASN 1052, wherein said modification comprises substitution of the amino acids with a lysine residue or an arginine residue. For example, in addition to a modification at M1144, said modified clostridial neurotoxin may comprise modifications of at least 5 amino acids selected from: ASN 886, ASN 930, ASN 954, SER 955, GLN 991, ASN 1025, ASN 1026, ASN 1052, and GLN 1229, wherein said modification comprises substitution of the amino acids with a lysine residue or an arginine residue.

Following the further modification, the further modified clostridial neurotoxin is capable of binding to the target cell receptors that unmodified BoNT/A (e.g. SEQ ID NO: 2) binds.

For a modified clostridial neurotoxin comprising the one or more further modifications, one way in which these advantageous properties (which represent an increase in the therapeutic index) may be defined is in terms of the Safety Ratio of the modified neurotoxin. In this regard, undesired effects of a clostridial neurotoxin (caused by diffusion of the toxin away from the site of administration) can be assessed experimentally by measuring percentage bodyweight loss in a relevant animal model (e.g. a mouse, where loss of bodyweight is detected within seven days of administration). Conversely, desired on-target effects of a clostridial neurotoxin can be assessed experimentally by Digital Abduction Score (DAS) assay, a measurement of muscle paralysis. The DAS assay may be performed by injection of 20 μl of clostridial neurotoxin, formulated in Gelatin Phosphate Buffer, into the mouse gastrocnemius/soleus complex, followed by assessment of Digital Abduction Score using the method of Aoki (Aoki K R, Toxicon 39: 1815-1820; 2001). In the DAS assay, mice are suspended briefly by the tail in order to elicit a characteristic startle response in which the mouse extends its hind limbs and abducts its hind digits. Following clostridial neurotoxin injection, the varying degrees of digit abduction are scored on a five-point scale (0=normal to 4=maximal reduction in digit abduction and leg extension).

The Safety Ratio of a clostridial neurotoxin may then be expressed as the ratio between the amount of toxin required for a 10% drop in bodyweight (measured at peak effect within the first seven days after dosing in a mouse) and the amount of toxin required for a DAS score of 2. High Safety Ratio scores are therefore desired and indicate a toxin that is able to effectively paralyse a target muscle with little undesired off-target effects. A modified clostridial neurotoxin comprising the one or more further modifications may have a Safety Ratio that is higher than the Safety Ratio of an equivalent unmodified (native) botulinum toxin (e.g. SEQ ID NO: 2) or when compared to a modified clostridial neurotoxin that does not comprise the one or more further modifications.

Thus, in one embodiment, a modified clostridial neurotoxin comprising the one or more further modifications has a Safety Ratio of at least 8 (for example, at least 8, 9, 10, 15, 20, 25, 30, 35, 40, 45 or 50), wherein Safety Ratio is calculated as: dose of toxin required for −10% bodyweight change (pg/mouse) divided by DAS ED₅₀ (pg/mouse) [ED₅₀=dose required to produce a DAS score of 2].

In one embodiment, a modified clostridial neurotoxin comprising the one or more further modifications has a Safety Ratio of at least 10. In one embodiment, a modified clostridial neurotoxin comprising the one or more further modifications has a Safety Ratio of at least 15.

The modified clostridial neurotoxins of the present invention are preferably free from the complexing proteins that are present in a naturally occurring clostridial neurotoxin complex.

Methods for modifying proteins by substitution, insertion or deletion of amino acid residues or via indels are known in the art. Byway of example, amino acid modifications may be introduced by modification of a nucleic acid sequence (e.g. DNA sequence) encoding a polypeptide (e.g. encoding an unmodified BoNT/A H_{cc} domain). This can be achieved using standard molecular cloning techniques, for example by site-directed mutagenesis where short strands of DNA (oligonucleotides) coding for the desired amino acid(s) are used to replace the original coding sequence using a polymerase enzyme, or by inserting/deleting parts of the gene with various enzymes (e.g., ligases and restriction endonucleases). Alternatively, a modified gene sequence can be chemically synthesised.

Thus, in one aspect, the invention provides a method for producing a modified clostridial neurotoxin, the method comprising:
(a) providing a first nucleic acid encoding (at least) a botulinum neurotoxin A (BoNT/A) H_{cc} domain and modifying the first nucleic acid to introduce a modification at methionine 1144 (M1144) of the encoded H_{cc} domain, thereby producing a second nucleic acid, wherein the modification increases oxidative resistance of the modified clostridial neurotoxin when compared to an otherwise identical clostridial neurotoxin lacking the modification; or
(b) synthesizing a nucleic acid that encodes (at least) a modified clostridial neurotoxin comprising a botulinum neurotoxin A (BoNT/A) H_{cc} domain, wherein the H_{cc} domain comprises a modification of methionine 1144 (M1144), and wherein the modification increases oxidative resistance of the modified clostridial neurotoxin when compared to an otherwise identical clostridial neurotoxin lacking the modification, thereby providing a synthesized nucleic acid; and
(c) expressing the second nucleic acid or the synthesized nucleic acid, respectively, thereby producing the modified clostridial neurotoxin; and
(d) optionally isolating the modified clostridial neurotoxin; and
(e) optionally activating the modified clostridial neurotoxin by contacting the (single-chain) modified clostridial neurotoxin with a protease that cleaves the (single-chain) modified clostridial neurotoxin at a recognition site (cleavage site) located between the light chain and heavy chain, thereby converting the (single-chain) modified clostridial neurotoxin into a di-chain modified clostridial neurotoxin, wherein the light chain and heavy chain are joined together by a disulphide bond.

For example, a method for producing a modified clostridial neurotoxin may comprise:
(a) providing a first nucleic acid encoding (at least) a botulinum neurotoxin A (BoNT/A) H_{cc} domain and modifying the first nucleic acid to introduce a modification at methionine 1144 (M1144) of the encoded H_{cc} domain, thereby producing a second nucleic acid, wherein the modification increases oxidative resistance of the modified clostridial neurotoxin when compared to an otherwise identical clostridial neurotoxin lacking the modification;
(b) expressing the second nucleic acid, thereby producing the modified clostridial neurotoxin; and
(c) optionally isolating the modified clostridial neurotoxin; and
(d) optionally activating the modified clostridial neurotoxin by contacting the (single-chain) modified clostridial neurotoxin with a protease that cleaves the (single-chain) modified clostridial neurotoxin at a recognition site (cleavage site) located between the light chain and heavy chain, thereby converting the (single-chain) modified clostridial neurotoxin into a di-chain modified clostridial neurotoxin, wherein the light chain and heavy chain are joined together by a disulphide bond.

For example, a method for producing a modified clostridial neurotoxin may comprise:
(a) synthesizing a nucleic acid that encodes (at least) a botulinum neurotoxin A (BoNT/A) H_{cc} domain, wherein the H_{cc} domain comprises a modification of methionine 1144 (M1144), and wherein the modification increases oxidative resistance of the modified clostridial neurotoxin when compared to an otherwise identical clostridial neurotoxin lacking the modification, thereby providing a synthesized nucleic acid;
(b) expressing the synthesized nucleic acid, thereby producing the modified clostridial neurotoxin; and
(c) optionally isolating the modified clostridial neurotoxin; and
(d) optionally activating the modified clostridial neurotoxin by contacting the (single-chain) modified clostridial neurotoxin with a protease that cleaves the (single-chain) modified clostridial neurotoxin at a recognition site (cleavage site) located between the light chain and heavy chain, thereby converting the (single-chain) modified clostridial neurotoxin into a di-chain modified clostridial neurotoxin, wherein the light chain and heavy chain are joined together by a disulphide bond.

The method may comprise:
(a) providing a first nucleic acid encoding (at least) a botulinum neurotoxin A (BoNT/A) H_{cc} domain and modifying the first nucleic acid to introduce a modification at methionine 1144 (M1144) of the encoded H_{cc} domain, thereby producing a second nucleic acid, wherein the modification increases oxidative resistance of the modified clostridial neurotoxin when compared to an otherwise identical clostridial neurotoxin lacking the modification; or
(b) synthesizing a nucleic acid that encodes (at least) a botulinum neurotoxin A (BoNT/A) H_{cc} domain, wherein the H_{cc} domain comprises a modification of methionine 1144 (M1144), and wherein the modification increases oxidative resistance of the modified clostridial neurotoxin when compared to an otherwise identical clostridial neurotoxin lacking the modification, thereby providing a synthesized nucleic acid; and
(c) expressing the second nucleic acid or the synthesized nucleic acid, respectively, thereby producing a single-chain modified clostridial neurotoxin; and
(d) isolating the single-chain modified clostridial neurotoxin.

The method may comprise:
(a) providing a first nucleic acid encoding (at least) a botulinum neurotoxin A (BoNT/A) H_{cc} domain and modifying the first nucleic acid to introduce a modification at methionine 1144 (M1144) of the encoded H_{cc} domain, thereby producing a second nucleic acid, wherein the modification increases oxidative resistance of the modified clostridial neurotoxin when compared to an otherwise identical clostridial neurotoxin lacking the modification; or
(b) synthesizing a nucleic acid that encodes (at least) a botulinum neurotoxin A (BoNT/A) H_{cc} domain, wherein the H_{cc} domain comprises a modification of methionine 1144 (M1144), and wherein the modification increases oxidative resistance of the modified clostridial neurotoxin when compared to an otherwise identical clostridial neurotoxin lacking the modification, thereby providing a synthesized nucleic acid; and
(c) expressing the second nucleic acid or the synthesized nucleic acid, respectively, thereby producing a single-chain modified clostridial neurotoxin; and
(d) activating the single-chain modified clostridial neurotoxin by contacting the single-chain modified clostridial neurotoxin with a protease that cleaves the single-chain modified clostridial neurotoxin at a recognition site (cleavage site) located between the light chain and heavy chain, thereby converting the single-chain modified clostridial neurotoxin into a di-chain modified clostridial neurotoxin, wherein the light chain and heavy chain are joined together by a disulphide bond.

Preferably, the di-chain modified clostridial neurotoxin is then isolated.

Preferably, the method may comprise:
(a) providing a first nucleic acid encoding (at least) a botulinum neurotoxin A (BoNT/A) H_{cc} domain and modifying the first nucleic acid to introduce a modification at methionine 1144 (M1144) of the encoded H_{cc} domain, thereby producing a second nucleic acid, wherein the modification increases oxidative resistance of the modified clostridial neurotoxin when compared to an otherwise identical clostridial neurotoxin lacking the modification; or
(b) synthesizing a nucleic acid that encodes (at least) a botulinum neurotoxin A (BoNT/A) H_{cc} domain, wherein the H_{cc} domain comprises a modification of methionine 1144 (M1144), and wherein the modification increases oxidative resistance of the modified clostridial neurotoxin when compared to an otherwise identical clostridial neurotoxin lacking the modification, thereby providing a synthesized nucleic acid; and
(c) expressing the second nucleic acid or the synthesized nucleic acid, respectively, thereby producing a single-chain modified clostridial neurotoxin; and (d) isolating the single-chain modified clostridial neurotoxin; and
(e) activating the single-chain modified clostridial neurotoxin by contacting the single-chain modified clostridial neurotoxin with a protease that cleaves the single-chain modified clostridial neurotoxin at a recognition site (cleavage site) located between the light chain and heavy chain, thereby converting the single-chain modified clostridial neurotoxin into a di-chain modified clostridial neurotoxin, wherein the light chain and heavy chain are joined together by a disulphide bond.

The invention also provides corresponding methods for increasing the oxidative resistance of a clostridial neurotoxin. Thus, in one aspect, the invention provides a method for increasing the oxidative resistance of a clostridial neurotoxin, the method comprising:
(a) providing a first nucleic acid encoding (at least) a botulinum neurotoxin A (BoNT/A) H_{cc} domain and modifying the first nucleic acid to introduce a modification at methionine 1144 (M1144) of the encoded H_{cc} domain, thereby producing a second nucleic acid, wherein the modification increases oxidative resistance of the modified clostridial neurotoxin when compared to an otherwise identical clostridial neurotoxin lacking the modification; or
(b) synthesizing a nucleic acid that encodes (at least) a modified clostridial neurotoxin comprising a botulinum neurotoxin A (BoNT/A) H_{cc} domain, wherein the H_{cc} domain comprises a modification of methionine 1144 (M1144), and wherein the modification increases oxidative resistance of the modified clostridial neurotoxin when compared to an otherwise identical clostridial neurotoxin lacking the modification, thereby providing a synthesized nucleic acid; and
(c) expressing the second nucleic acid or the synthesized nucleic acid, respectively, thereby producing the modified clostridial neurotoxin; and (d) optionally isolating the modified clostridial neurotoxin; and
(e) optionally activating the modified clostridial neurotoxin by contacting the (single-chain) modified clostridial neurotoxin with a protease that cleaves the (single-chain) modified clostridial neurotoxin at a recognition site (cleavage site) located between the light chain and heavy chain, thereby converting the (single-chain) modified clostridial neurotoxin into a di-chain modified clostridial neurotoxin, wherein the light chain and heavy chain are joined together by a disulphide bond.

Preferably, the di-chain modified clostridial neurotoxin is isolated.

The second nucleic acid or synthesized nucleic acid preferably encodes a modified clostridial neurotoxin comprising a botulinum neurotoxin A (BoNT/A) H_{cc} domain. Thus, said nucleic acid may encode a clostridial neurotoxin L-chain and H-chain, said H-chain comprising a translocation domain and an He domain, wherein the He domain comprises the H_{cc} domain modified at M1144.

The invention also provides a modified clostridial neurotoxin obtainable by a method of the invention. The term “obtainable” as used herein encompasses the term “obtained”. In one embodiment “obtainable” means “obtained”.

A first nucleic acid for use in a method of the invention may comprise a nucleic acid sequence that encodes an unmodified BoNT/A H_{cc} domain described herein. The first nucleic acid may comprise a nucleic acid that encodes a clostridial neurotoxin (e.g. comprising a light-chain and heavy-chain) comprising methionine at position 1144. Preferably, a first nucleic acid comprises a nucleic acid sequence that encodes an unmodified BoNT/A described herein.

In one embodiment, a first nucleic acid comprises a nucleic acid having at least 70% sequence identity to any one of SEQ ID NOs: 1, 10, 19, 28 or 37. In one embodiment, a first nucleic acid comprises a nucleic acid having at least 80%, 90%, 95%, or 98% sequence identity to any one of SEQ ID NOs: 1, 10, 19, 28 or 37. Preferably, a first nucleic acid comprises (more preferably consists of) any one of SEQ ID NOs: 1, 10, 19, 28 or 37. Of the sequences indicated, SEQ ID NO: 10 is most preferred.

In one embodiment, the second nucleic acid or synthesized nucleic acid comprises a nucleic acid having at least 70% sequence identity to any one of SEQ ID NOs: 136-138. In one embodiment, the second nucleic acid or synthesized nucleic acid comprises a nucleic acid having at least 80%, 90%, 95%, or 98% sequence identity to any one of SEQ ID NOs: 136-138. Preferably, the second nucleic acid or synthesized nucleic acid comprises (more preferably consists of) any one of SEQ ID NOs: 136-138.

In one embodiment the modification comprises replacing a codon encoding for M1144 with a codon encoding for valine (e.g. GTG), leucine (e.g. CTG) or glycine (e.g. GGT).

In a related aspect, the invention provides a nucleic acid (preferably DNA) sequence encoding a modified clostridial neurotoxin of the invention. In one embodiment, the nucleic acid sequence is prepared as part of a DNA vector comprising a promoter and a terminator.

In one embodiment, the nucleic acid comprises a nucleic acid having at least 70% sequence identity to any one of SEQ ID NOs: 136-138. In one embodiment, the nucleic acid comprises a nucleic acid having at least 80%, 90%, 95%, or 98% sequence identity to any one of SEQ ID NOs: 136-138. Preferably, the nucleic acid comprises (more preferably consists of) any one of SEQ ID NOs: 136-138.

The modified clostridial neurotoxins of the present invention can be produced using recombinant nucleic acid technologies. Thus, in one embodiment, a modified clostridial neurotoxin (as described above) is a recombinant modified clostridial neurotoxin.

In a preferred embodiment, the vector has a promoter selected from:

| Promoter | Induction Agent | Typical Induction Condition |
|---|---|---|
| Tac (hybrid) | IPTG | 0.2 mM (0.05-2.0 mM) |
| AraBAD | L-arabinose | 0.2% (0.002-0.4%) |
| T7-lac operator | IPTG | 0.2 mM (0.05-2.0 mM) |

In another preferred embodiment, the vector has a promoter selected from:

| Promoter | Induction Agent | Typical Induction Condition |
|---|---|---|
| Tac (hybrid) | IPTG | 0.2 mM (0.05-2.0 mM) |
| AraBAD | L-arabinose | 0.2% (0.002-0.4%) |
| T7-lac operator | IPTG | 0.2 mM (0.05-2.0 mM) |
| T5-lac operator | IPTG | 0.2 mM (0.05-2.0 mM) |

The nucleic acid molecules of the invention may be made using any suitable process known in the art. Thus, the nucleic acid molecules may be made using chemical synthesis techniques. Alternatively, the nucleic acid molecules of the invention may be made using molecular biology techniques.

For example, a nucleic acid of the present invention may be designed in silico, and then synthesised by conventional synthesis techniques.

The above-mentioned nucleic acid sequence information is optionally modified for codon-biasing according to the ultimate host cell (e.g. Escherichia coli) expression system that is to be employed.

The terms “nucleotide sequence” and “nucleic acid” are used synonymously herein. Preferably the nucleotide sequence is a DNA sequence.

A nucleic acid sequence described herein may be expressed to produce a modified clostridial neurotoxin using any technique known in the art. For example, a nucleic acid may be expressed in a cell-free in vitro system. Alternatively, a nucleic acid is preferably expressed in a suitable host cell, such as an Escherichia coli host cell.

Thus, in one aspect, the invention provides a method for producing a single-chain modified clostridial neurotoxin having a light chain and a heavy chain, the method comprising expressing a nucleic acid of the invention in a suitable host cell, lysing the host cell to provide a host cell homogenate containing the single-chain modified clostridial neurotoxin, and isolating the single-chain modified clostridial neurotoxin.

Subsequent to expression, a modified clostridial neurotoxin may be isolated. Isolating a modified clostridial neurotoxin can be achieved by any purification methods, such as chromatographic or immunoaffinity methods, known to the person skilled in the art. A purification tag can assist in isolation. Thus, a modified clostridial neurotoxin described herein may comprise one or more tags (e.g. purification tags), such as a His-tag or Strep-tag. It is preferred that the modified clostridial neurotoxins do not comprise such tags or that the tag is removed before use. The modified clostridial neurotoxins may also comprise one or more cleavage sites, such as a TEV cleavage site, to facilitate removal of a tag.

A modified clostridial neurotoxin may be converted from a single-chain polypeptide to a corresponding di-chain polypeptide at any point during production. However, it is preferred that this is carried out after isolating the modified clostridial neurotoxin.

Thus, in one aspect, the invention provides a method of activating a modified clostridial neurotoxin, the method comprising providing a single-chain modified clostridial neurotoxin obtainable by a method of the invention, contacting the single-chain modified clostridial neurotoxin with a protease that cleaves the single-chain modified clostridial neurotoxin at a recognition site (cleavage site) located between the light chain and heavy chain, thereby converting the single-chain modified clostridial neurotoxin into a di-chain modified clostridial neurotoxin, wherein the light chain and heavy chain are joined together by a disulphide bond.

The present invention therefore provides a di-chain clostridial neurotoxin obtainable by a method of the invention.

Activation is preferably carried out by contacting the modified clostridial neurotoxin with Lys-C as described in WO 2014/080206 A1.

As discussed above, clostridial neurotoxins are formed from two polypeptide chains, the heavy chain (H-chain), which has a molecular mass of approximately 100 kDa, and the light chain (L-chain), which has a molecular mass of approximately 50 kDa. The H-chain comprises a C-terminal targeting component (receptor binding domain or He domain) and an N-terminal translocation component (H_{N} domain).

Examples of light chain reference sequences include:
Botulinum type A neurotoxin: amino acid residues 1-448
Botulinum type B neurotoxin: amino acid residues 1-440
Botulinum type C1 neurotoxin: amino acid residues 1-441
Botulinum type D neurotoxin: amino acid residues 1-445
Botulinum type E neurotoxin: amino acid residues 1-422
Botulinum type F neurotoxin: amino acid residues 1-439
Botulinum type G neurotoxin: amino acid residues 1-441
Tetanus neurotoxin: amino acid residues 1-457

For recently-identified BoNT/X, the L-chain has been reported as corresponding to amino acids 1-439 thereof, with the L-chain boundary potentially varying by approximately 25 amino acids (e.g. 1-414 or 1-464).

The above-identified reference sequences should be considered a guide, as slight variations may occur according to sub-serotypes. By way of example, US 2007/0166332 (hereby incorporated by reference in its entirety) cites slightly different clostridial sequences:
Botulinum type A neurotoxin: amino acid residues M1-K448
Botulinum type B neurotoxin: amino acid residues M1-K441
Botulinum type C1 neurotoxin: amino acid residues M1-K449
Botulinum type D neurotoxin: amino acid residues M1-R445
Botulinum type E neurotoxin: amino acid residues M1-R422
Botulinum type F neurotoxin: amino acid residues M1-K439
Botulinum type G neurotoxin: amino acid residues M1-K446
Tetanus neurotoxin: amino acid residues M1-A457

A Translocation Domain is a molecule that enables translocation of a protease into a target cell such that a functional expression of protease activity occurs within the cytosol of the target cell. Whether any molecule (e.g. a protein or peptide) possesses the requisite translocation function of the present invention may be confirmed by any one of a number of conventional assays.

For example, Shone C. (1987) describes an in vitro assay employing liposomes, which are challenged with a test molecule. Presence of the requisite translocation function is confirmed by release from the liposomes of K⁺ and/or labelled NAD, which may be readily monitored [see Shone C. (1987) Eur. J. Biochem; vol. 167(1): pp. 175-180].

A further example is provided by Blaustein R. (1987), which describes a simple in vitro assay employing planar phospholipid bilayer membranes. The membranes are challenged with a test molecule and the requisite translocation function is confirmed by an increase in conductance across said membranes [see Blaustein (1987) FEBS Letts; vol. 226, no. 1: pp. 115-120].

Additional methodology to enable assessment of membrane fusion and thus identification of Translocation Domains suitable for use in the present invention are provided by Methods in Enzymology Vol 220 and 221, Membrane Fusion Techniques, Parts A and B, Academic Press 1993.

The present invention also embraces variant translocation domains, so long as the variant domains still demonstrate the requisite translocation activity. By way of example, a variant may have at least 70%, preferably at least 80%, more preferably at least 90%, and most preferably at least 95% or at least 98% amino acid sequence homology with a reference translocation domain. The term fragment, when used in relation to a translocation domain, means a peptide having at least 20, preferably at least 40, more preferably at least 80, and most preferably at least 100 amino acid residues of the reference translocation domain. In the case of a clostridial translocation domain, the fragment preferably has at least 100, preferably at least 150, more preferably at least 200, and most preferably at least 250 amino acid residues of the reference translocation domain (e.g. H_{N} domain). Translocation ‘fragments’ of the present invention embrace fragments of variant translocation domains based on the reference sequences.

The Translocation Domain is preferably capable of formation of ion-permeable pores in lipid membranes under conditions of low pH. Preferably it has been found to use only those portions of the protein molecule capable of pore-formation within the endosomal membrane.

The Translocation Domain may be obtained from a microbial protein source, in particular from a bacterial or viral protein source. Hence, in one embodiment, the Translocation Domain is a translocating domain of an enzyme, such as a bacterial toxin or viral protein.

It is well documented that certain domains of bacterial toxin molecules are capable of forming such pores. It is also known that certain translocation domains of virally expressed membrane fusion proteins are capable of forming such pores. Such domains may be employed in the present invention.

The Translocation Domain may be of a clostridial origin, such as the H_{N} domain (or a functional component thereof). H_{N} means a portion or fragment of the H-chain of a clostridial neurotoxin approximately equivalent to the amino-terminal half of the H-chain, or the domain corresponding to that fragment in the intact H-chain.

Examples of suitable (reference) Translocation Domains include:
Botulinum type A neurotoxin—amino acid residues (449-871)
Botulinum type B neurotoxin—amino acid residues (441-858)
Botulinum type C neurotoxin—amino acid residues (442-866)
Botulinum type D neurotoxin—amino acid residues (446-862)
Botulinum type E neurotoxin—amino acid residues (423-845)
Botulinum type F neurotoxin—amino acid residues (440-864)
Botulinum type G neurotoxin—amino acid residues (442-863)
Tetanus neurotoxin—amino acid residues (458-879)

The above-identified reference sequence should be considered a guide as slight variations may occur according to sub-serotypes. By way of example, US 2007/0166332 (hereby incorporated by reference thereto) cites slightly different clostridial sequences:
Botulinum type A neurotoxin—amino acid residues (A449-K871)
Botulinum type B neurotoxin—amino acid residues (A442-S858)
Botulinum type C neurotoxin—amino acid residues (T450-N866)
Botulinum type D neurotoxin—amino acid residues (D446-N862)
Botulinum type E neurotoxin—amino acid residues (K423-K845)
Botulinum type F neurotoxin—amino acid residues (A440-K864)
Botulinum type G neurotoxin—amino acid residues (S447-S863)
Tetanus neurotoxin—amino acid residues (S458-V879)

In the context of the present invention, a variety of clostridial neurotoxin H_{N} regions comprising a translocation domain can be useful in aspects of the present invention with the proviso that these active fragments can facilitate the release of a non-cytotoxic protease (e.g. a clostridial L-chain) from intracellular vesicles into the cytoplasm of the target cell and thus participate in executing the overall cellular mechanism whereby a clostridial neurotoxin proteolytically cleaves a substrate. The H_{N} regions from the heavy chains of clostridial neurotoxins are approximately 410-430 amino acids in length and comprise a translocation domain. Research has shown that the entire length of a H_{N} region from a clostridial neurotoxin heavy chain is not necessary for the translocating activity of the translocation domain. Thus, aspects of this embodiment can include clostridial neurotoxin H_{N} regions comprising a translocation domain having a length of, for example, at least 350 amino acids, at least 375 amino acids, at least 400 amino acids and at least 425 amino acids. Other aspects of this embodiment can include clostridial neurotoxin H_{N} regions comprising translocation domain having a length of, for example, at most 350 amino acids, at most 375 amino acids, at most 400 amino acids and at most 425 amino acids.

For further details on the genetic basis of toxin production in Clostridium botulinum and C. tetani, we refer to Henderson et al (1997) in The Clostridia: Molecular Biology and Pathogenesis, Academic press.

The term H_{N} embraces naturally-occurring neurotoxin H_{N} portions, and modified H_{N} portions having amino acid sequences that do not occur in nature and/or synthetic amino acid residues, so long as the modified H_{N} portions still demonstrate the above-mentioned translocation function.

Alternatively, the Translocation Domain may be of a non-clostridial origin. Examples of non-clostridial (reference) Translocation Domain origins include, but not be restricted to, the translocation domain of diphtheria toxin [O'Keefe et al., Proc. Natl. Acad. Sci. USA (1992) 89, 6202-6206; Silverman et al., J. Biol. Chem. (1993) 269, 22524-22532; and London, E. (1992) Biochem. Biophys. Acta., 1112, pp. 25-51], the translocation domain of Pseudomonas exotoxin type A [Prior et al. Biochemistry (1992) 31, 3555-3559], the translocation domains of anthrax toxin [Blanke et al. Proc. Natl. Acad. Sci. USA (1996) 93, 8437-8442], a variety of fusogenic or hydrophobic peptides of translocating function [Plank et al. J. Biol. Chem. (1994) 269, 12918-12924; and Wagner et al (1992) PNAS, 89, pp. 7934-7938], and amphiphilic peptides [Murata et al (1992) Biochem., 31, pp. 1986-1992]. The Translocation Domain may mirror the Translocation Domain present in a naturally-occurring protein, or may include amino acid variations so long as the variations do not destroy the translocating ability of the Translocation Domain.

Particular examples of viral (reference) Translocation Domains suitable for use in the present invention include certain translocating domains of virally expressed membrane fusion proteins. For example, Wagner et al. (1992) and Murata et al. (1992) describe the translocation (i.e. membrane fusion and vesiculation) function of a number of fusogenic and amphiphilic peptides derived from the N-terminal region of influenza virus haemagglutinin. Other virally expressed membrane fusion proteins known to have the desired translocating activity are a translocating domain of a fusogenic peptide of Semliki Forest Virus (SFV), a translocating domain of vesicular stomatitis virus (VSV) glycoprotein G, a translocating domain of SER virus F protein and a translocating domain of Foamy virus envelope glycoprotein. Virally encoded Aspike proteins have particular application in the context of the present invention, for example, the E1 protein of SFV and the G protein of the G protein of VSV.

Use of the (reference) Translocation Domains listed in the table (below) includes use of sequence variants thereof. A variant may comprise one or more conservative nucleic acid substitutions and/or nucleic acid deletions or insertions, with the proviso that the variant possesses the requisite translocating function. A variant may also comprise one or more amino acid substitutions and/or amino acid deletions or insertions, so long as the variant possesses the requisite translocating function.

| Translocation | | |
|---|---|---|
| Domain source | Amino acid residues | References |
| Diphtheria toxin | 194-380 | Silverman et al., 1994, J. Biol. Chem. |
| | | 269, 22524-22532 |
| | | London E., 1992, Biochem. Biophys. |
| | | Acta., 1113, 25-51 |
| Domain II of | 405-613 | Prior et al., 1992, Biochemistry 31, 3555- |
| pseudomonas | | 3559 |
| exotoxin | | Kihara & Pastan, 1994, Bioconj Chem. 5, |
| | | 532-538 |
| Influenza virus | GLFGAIAGFIENGWE | Plank et al., 1994, J. Biol. Chem. 269, |
| haemagglutinin | GMIDGWYG (SEQ ID | 12918-12924 |
| | NO: 116), and | Wagner et al., 1992, PNAS, 89, 7934- |
| | Variants thereof | 7938 |
| | | Murata et al., 1992, Biochemistry 31, |
| | | 1986-1992 |
| Semliki Forest virus | Translocation domain | Kielian et al., 1996, J Cell Biol. 134(4), |
| fusogenic protein | | 863-872 |
| Vesicular Stomatitis | 118-139 | Yao et al., 2003, Virology 310(2), 319-332 |
| virus glycoprotein | | |
| G | | |
| SER virus F protein | Translocation domain | Seth et al., 2003, J Virol 77(11) 6520- |
| | | 6527 |
| Foamy virus | Translocation domain | Picard-Maureau et al., 2003, J Virol. |
| envelope | | 77(8), 4722-4730 |
| glycoprotein | | |

Examples of clostridial neurotoxin He domain reference sequences include:
BoNT/A—N872-L1296
BoNT/B—E859-E1291
BoNT/C1—N867-E1291
BoNT/D—S863-E1276
BoNT/E—R846-K1252
BoNT/F—K865-E1274
BoNT/G—N864-E1297
TeNT—1880-D1315

For recently-identified BoNT/X, the He domain has been reported as corresponding to amino acids 893-1306 thereof, with the domain boundary potentially varying by approximately 25 amino acids (e.g. 868-1306 or 918-1306).

The clostridial neurotoxins described herein may further comprise a translocation facilitating domain. Said domain facilitates delivery of the non-cytotoxic protease into the cytosol of the target cell and are described, for example, in WO 08/008803 and WO 08/008805, each of which is herein incorporated by reference thereto.

By way of example, suitable translocation facilitating domains include an enveloped virus fusogenic peptide domain, for example, suitable fusogenic peptide domains include influenzavirus fusogenic peptide domain (e.g. influenza A virus fusogenic peptide domain of 23 amino acids), alphavirus fusogenic peptide domain (e.g. Semliki Forest virus fusogenic peptide domain of 26 amino acids), vesiculovirus fusogenic peptide domain (e.g. vesicular stomatitis virus fusogenic peptide domain of 21 amino acids), respirovirus fusogenic peptide domain (e.g. Sendai virus fusogenic peptide domain of 25 amino acids), morbiliivirus fusogenic peptide domain (e.g. Canine distemper virus fusogenic peptide domain of 25 amino acids), avulavirus fusogenic peptide domain (e.g. Newcastle disease virus fusogenic peptide domain of 25 amino acids), henipavirus fusogenic peptide domain (e.g. Hendra virus fusogenic peptide domain of 25 amino acids), metapneumovirus fusogenic peptide domain (e.g. Human metapneumovirus fusogenic peptide domain of 25 amino acids) or spumavirus fusogenic peptide domain such as simian foamy virus fusogenic peptide domain; or fragments or variants thereof.

By way of further example, a translocation facilitating domain may comprise a clostridial neurotoxin H_{CN} domain or a fragment or variant thereof. In more detail, a clostridial neurotoxin H_{CN} translocation facilitating domain may have a length of at least 200 amino acids, at least 225 amino acids, at least 250 amino acids, at least 275 amino acids. In this regard, a clostridial neurotoxin H_{CN} translocation facilitating domain preferably has a length of at most 200 amino acids, at most 225 amino acids, at most 250 amino acids, or at most 275 amino acids. Specific (reference) examples include:
Botulinum type A neurotoxin—amino acid residues (872-1110)
Botulinum type B neurotoxin—amino acid residues (859-1097)
Botulinum type C neurotoxin—amino acid residues (867-1111)
Botulinum type D neurotoxin—amino acid residues (863-1098)
Botulinum type E neurotoxin—amino acid residues (846-1085)
Botulinum type F neurotoxin—amino acid residues (865-1105)
Botulinum type G neurotoxin—amino acid residues (864-1105)
Tetanus neurotoxin—amino acid residues (880-1127)

The above sequence positions may vary a little according to serotype/sub-type, and further examples of suitable (reference) clostridial neurotoxin H_{CN} domains include:
Botulinum type A neurotoxin—amino acid residues (874-1110)
Botulinum type B neurotoxin—amino acid residues (861-1097)
Botulinum type C neurotoxin—amino acid residues (869-1111)
Botulinum type D neurotoxin—amino acid residues (865-1098)
Botulinum type E neurotoxin—amino acid residues (848-1085)
Botulinum type F neurotoxin—amino acid residues (867-1105)
Botulinum type G neurotoxin—amino acid residues (866-1105)
Tetanus neurotoxin—amino acid residues (882-1127)

Any of the above-described facilitating domains may be combined with any of the previously described translocation domain peptides that are suitable for use in the present invention. Thus, by way of example, a non-clostridial facilitating domain may be combined with non-clostridial translocation domain peptide or with clostridial translocation domain peptide. Alternatively, a clostridial neurotoxin H_{CN} translocation facilitating domain may be combined with a non-clostridial translocation domain peptide. Alternatively, a clostridial neurotoxin H_{CN} facilitating domain may be combined or with a clostridial translocation domain peptide, examples of which include:
Botulinum type A neurotoxin—amino acid residues (449-1110)
Botulinum type B neurotoxin—amino acid residues (442-1097)
Botulinum type C neurotoxin—amino acid residues (450-1111)
Botulinum type D neurotoxin—amino acid residues (446-1098)
Botulinum type E neurotoxin—amino acid residues (423-1085)
Botulinum type F neurotoxin—amino acid residues (440-1105)
Botulinum type G neurotoxin—amino acid residues (447-1105)
Tetanus neurotoxin—amino acid residues (458-1127)

The He peptide of a native clostridial neurotoxin comprises approximately 400-440 amino acid residues, and consists of two functionally distinct domains of approximately 25 kDa each, namely the N-terminal region (commonly referred to as the H_{CN} peptide or domain) and the C-terminal region (commonly referred to as the H_{cc} peptide or domain). This fact is confirmed by the following publications, each of which is herein incorporated in its entirety by reference thereto: Umland T C (1997) Nat. Struct. Biol. 4: 788-792; Herreros J (2000) Biochem. J. 347: 199-204; Halpern J (1993) J. Biol. Chem. 268: 15, pp. 11188-11192; Rummel A (2007) PNAS 104: 359-364; Lacey DB (1998) Nat. Struct. Biol. 5: 898-902; Knapp (1998) Am. Cryst. Assoc. Abstract Papers 25: 90; Swaminathan and Eswaramoorthy (2000) Nat. Struct. Biol. 7: 1751-1759; and Rummel A (2004) Mol. Microbiol. 51(3), 631-643. Moreover, it has been well documented that the C-terminal region (H_{cc}), which constitutes the C-terminal 160-200 amino acid residues, is responsible for binding of a clostridial neurotoxin to its natural cell receptors, namely to nerve terminals at the neuromuscular junction—this fact is also confirmed by the above publications.

Example clostridial H_{cc} reference sequences are presented below:
Botulinum type A neurotoxin—amino acid residues (Y1111-L1296)
Botulinum type B neurotoxin—amino acid residues (Y1098-E1291)
Botulinum type C neurotoxin—amino acid residues (Y1112-E1291)
Botulinum type D neurotoxin—amino acid residues (Y1099-E1276)
Botulinum type E neurotoxin—amino acid residues (Y1086-K1252)
Botulinum type F neurotoxin—amino acid residues (Y1106-E1274)
Botulinum type G neurotoxin—amino acid residues (Y1106-E1297)
Tetanus neurotoxin—amino acid residues (Y1128-D1315).

The above-identified reference sequences should be considered a guide as slight variations may occur according to sub-serotypes.

A modified clostridial neurotoxin may have one or more further modifications in the amino acid sequence of the light chain, for example modifications in the substrate binding or catalytic domain which may alter or modify the SNARE protein specificity of the modified L-chain. Examples of such modified clostridial neurotoxins are described in WO 2010/120766 and US 2011/0318385, both of which are hereby incorporated by reference in their entirety.

A modified clostridial neurotoxin may comprise one or more further modifications that increases or decreases the biological activity and/or the biological persistence of the modified clostridial neurotoxin. For example, a modified clostridial neurotoxin may comprise a leucine- or tyrosine-based motif, wherein said motif increases or decreases the biological activity and/or the biological persistence of the modified clostridial neurotoxin. Suitable leucine-based motifs include xDxxxLL (SEQ ID NO: 117), xExxxLL (SEQ ID NO: 118), xExxxlL (SEQ ID NO: 119), and xExxxLM (SEQ ID NO: 120) (wherein x is any amino acid). Suitable tyrosine-based motifs include Y-x-x-Hy (SEQ ID NO: 121) (wherein Hy is a hydrophobic amino acid). Examples of modified clostridial neurotoxins comprising leucine- and tyrosine-based motifs are described in WO 2002/08268, which is hereby incorporated by reference in its entirety.

While it is preferred that a modified clostridial neurotoxin of the invention is a modified BoNT/A, the present invention is suitable for application to many different varieties of clostridial neurotoxins with the proviso that said clostridial neurotoxin comprises a modified BoNT/A H_{cc} domain of the invention (this proviso is, however, not essential in respect of the method for selecting an oxidation resistant clostridial neurotoxin, as discussed below). Thus, in the context of the present invention, the term “clostridial neurotoxin” embraces toxins produced by C. botulinum (botulinum neurotoxin serotypes A, B, C1, D, E, F, G, H, and X), C. tetani (tetanus neurotoxin), C. butyricum (botulinum neurotoxin serotype E), and C. baratii (botulinum neurotoxin serotype F), as well as modified clostridial neurotoxins or derivatives derived from any of the foregoing, with the proviso that said clostridial neurotoxin comprises a modified BoNT/A H_{cc} domain of the invention (this proviso is, however, not essential in respect of the method for selecting an oxidation resistant clostridial neurotoxin, as discussed below). The term “clostridial neurotoxin” also embraces botulinum neurotoxin serotype H with the proviso that said clostridial neurotoxin comprises a modified BoNT/A H_{cc} domain of the invention (this proviso is, however, not essential in respect of the method for selecting an oxidation resistant clostridial neurotoxin, as discussed below).

Thus, the term “clostridial neurotoxin” is intended to embrace hybrid and chimeric clostridial neurotoxins. In one embodiment a modified clostridial neurotoxin may be a hybrid or chimeric clostridial neurotoxin with the proviso that said clostridial neurotoxin comprises a modified BoNT/A H_{cc} domain of the invention (again, this proviso is not essential in respect of the method for selecting an oxidation resistant clostridial neurotoxin, as discussed below). A hybrid clostridial neurotoxin comprises at least a portion of a light chain from one clostridial neurotoxin or subtype thereof, and at least a portion of a heavy chain from another clostridial neurotoxin or clostridial neurotoxin subtype. In one embodiment the hybrid clostridial neurotoxin may contain the entire light chain from one clostridial neurotoxin subtype and the heavy chain from another clostridial neurotoxin subtype. In another embodiment, a chimeric clostridial neurotoxin may contain a portion (e.g. the binding domain) of the heavy chain of one clostridial neurotoxin subtype, with another portion of the heavy chain being from another clostridial neurotoxin subtype. Similarly or alternatively, the therapeutic element may comprise light chain portions from different clostridial neurotoxins. Such hybrid or chimeric clostridial neurotoxins are useful, for example, as a means of delivering the therapeutic benefits of such clostridial neurotoxins to patients who are immunologically resistant to a given clostridial neurotoxin subtype, to patients who may have a lower than average concentration of receptors to a given clostridial neurotoxin heavy chain binding domain, or to patients who may have a protease-resistant variant of the membrane or vesicle toxin substrate (e.g., SNAP-25, VAMP and syntaxin). Hybrid and chimeric clostridial neurotoxins are described in U.S. Pat. No. 8,071,110, which publication is hereby incorporated by reference in its entirety.

Botulinum neurotoxin (BoNT) is produced by C. botulinum in the form of a large protein complex, consisting of BoNT itself complexed to a number of accessory proteins. There are at present nine different classes of botulinum neurotoxin, namely: botulinum neurotoxin serotypes A, B, C1, D, E, F, G, H, and X all of which share similar structures and modes of action. Different BoNT serotypes can be distinguished based on inactivation by specific neutralising anti-sera, with such classification by serotype correlating with percentage sequence identity at the amino acid level. BoNT proteins of a given serotype are further divided into different subtypes on the basis of amino acid percentage sequence identity.

Unmodified BoNT/A polypeptide sequences (including unmodified BoNT/A1, BoNT/A3, and BoNT/A4) are described above.

A BoNT/B may comprise a polypeptide sequence having at least 70% sequence identity to SEQ ID NO: 128. For example, a BoNT/B may comprise a polypeptide sequence having at least 80%, 90%, 95%, or 98% sequence identity to SEQ ID NO: 128. Preferably BoNT/B comprises (or consists of) SEQ ID NO: 128.

A BoNT/C1 may comprise a polypeptide sequence having at least 70% sequence identity to SEQ ID NO: 129. For example, a BoNT/C1 may comprise a polypeptide sequence having at least 80%, 90%, 95%, or 98% sequence identity to SEQ ID NO: 129. Preferably BoNT/C1 comprises (or consists of) SEQ ID NO: 129.

A BoNT/D may comprise a polypeptide sequence having at least 70% sequence identity to SEQ ID NO: 130. For example, a BoNT/D may comprise a polypeptide sequence having at least 80%, 90%, 95%, or 98% sequence identity to SEQ ID NO: 130. Preferably BoNT/D comprises (or consists of) SEQ ID NO: 130.

A BoNT/E may comprise a polypeptide sequence having at least 70% sequence identity to SEQ ID NO: 131. For example, a BoNT/E may comprise a polypeptide sequence having at least 80%, 90%, 95%, or 98% sequence identity to SEQ ID NO: 131. Preferably BoNT/E comprises (or consists of) SEQ ID NO: 131.

A BoNT/F may comprise a polypeptide sequence having at least 70% sequence identity to SEQ ID NO: 132. For example, a BoNT/F may comprise a polypeptide sequence having at least 80%, 90%, 95%, or 98% sequence identity to SEQ ID NO: 132. Preferably BoNT/F comprises (or consists of) SEQ ID NO: 132.

A BoNT/G may comprise a polypeptide sequence having at least 70% sequence identity to SEQ ID NO: 133. For example, a BoNT/G may comprise a polypeptide sequence having at least 80%, 90%, 95%, or 98% sequence identity to SEQ ID NO: 133. Preferably BoNT/G comprises (or consists of) SEQ ID NO: 133.

A TeNT may comprise a polypeptide sequence having at least 70% sequence identity to SEQ ID NO: 134. For example, a TeNT may comprise a polypeptide sequence having at least 80%, 90%, 95%, or 98% sequence identity to SEQ ID NO: 134. Preferably TeNT comprises (or consists of) SEQ ID NO: 134.

A BoNT/X may comprise a polypeptide sequence having at least 70% sequence identity to SEQ ID NO: 135. For example, a BoNT/X may comprise a polypeptide sequence having at least 80%, 90%, 95%, or 98% sequence identity to SEQ ID NO: 135. Preferably BoNT/X comprises (or consists of) SEQ ID NO: 135.

BoNTs are absorbed in the gastrointestinal tract, and, after entering the general circulation, bind to the presynaptic membrane of cholinergic nerve terminals and prevent the release of their neurotransmitter acetylcholine. BoNT/B, BoNT/D, BoNT/F and BoNT/G cleave synaptobrevin/vesicle-associated membrane protein (VAMP); BoNT/C1, BoNT/A and BoNT/E cleave the synaptosomal-associated protein of 25 kDa (SNAP-25); and BoNT/C1 cleaves syntaxin. BoNT/X has been found to cleave SNAP-25, VAMP1, VAMP2, VAMP3, VAMP4, VAMP5, Ykt6, and syntaxin 1.

Tetanus toxin is produced in a single serotype by C. tetani. C. butyricum produces BoNT/E, while C. baratii produces BoNT/F.

The term “clostridial neurotoxin” may also embrace newly discovered botulinum neurotoxin protein family members expressed by non-clostridial microorganisms, such as the Enterococcus encoded toxin which has closest sequence identity to BoNT/X, the Weissella oryzae encoded toxin called BoNT/Wo (NCBI Ref Seq: WP_027699549.1), which cleaves VAMP2 at W89-W90, the Enterococcus faecium encoded toxin (GenBank: OTO2244.1), which cleaves VAMP2 and SNAP25, and the Chryseobacterium pipero encoded toxin (NCBI Ref.Seq: WP_034687872.1), with the proviso that said clostridial neurotoxin comprises a modified BoNT/A H_{cc} domain of the invention (again, this proviso is not essential in respect of the method for selecting an oxidation resistant clostridial neurotoxin, as discussed below).

A modified clostridial neurotoxin of the invention may comprise a BoNT/A H_{cc} domain modified as described herein and:
(i) a BoNT/A L-chain and/or a BoNT/A H_{N} domain (preferably a BoNT/A L-chain and a BoNT/A H_{N} domain);
(ii) a BoNT/B L-chain and/or a BoNT/B H_{N} domain (preferably a BoNT/B L-chain and a BoNT/B H_{N} domain);
(iii) a BoNT/C1 L-chain and/or a BoNT/C1 H_{N} domain (preferably a BoNT/C1 L-chain and a BoNT/C1 H_{N} domain);
(iv) a BoNT/D L-chain and/or a BoNT/D H_{N} domain (preferably a BoNT/D L-chain and a BoNT/D H_{N} domain);
(v) a BoNT/E L-chain and/or a BoNT/E H_{N} domain (preferably a BoNT/E L-chain and a BoNT/E H_{N} domain);
(vi) a BoNT/F L-chain and/or a BoNT/F H_{N} domain (preferably a BoNT/F L-chain and a BoNT/F H_{N} domain);
(iv) a BoNT/G L-chain and/or a BoNT/G H_{N} domain (preferably a BoNT/G L-chain and a BoNT/G H_{N} domain);
(iv) a BoNT/X L-chain and/or a BoNT/X H_{N} domain (preferably a BoNT/X L-chain and a BoNT/X H_{N} domain); or
(iv) a TeNT L-chain and/or a TeNT H_{N} domain (preferably a TeNT L-chain and a TeNT H_{N} domain).

The modified clostridial neurotoxin preferably comprises a BoNT/A He domain comprising the BoNT/A H_{cc} domain modified as described herein. The BoNT/A H_{cc} domain modified as described herein may replace the native BoNT/A He domain. The BoNT/A He domain may be from the same or different (preferably the same) BoNT/A sub-serotype (e.g. BoNT/A1, BoNT/A3 or BoNT/A4) as the H_{cc} domain. This is preferred when the modified clostridial neurotoxin comprises a BoNT/A L-chain and/or H_{N} domain as described above.

Alternatively, the modified clostridial neurotoxin may comprise a BoNT/B, BoNT/C1, BoNT/D, BoNT/E, BoNT/F, BoNT/G, BoNT/X, or TeNT He domain in which the H_{cc} domain thereof has been replaced with the BoNT/A H_{cc} domain modified as described herein. This is preferred when the modified clostridial neurotoxin comprises a BoNT/B, BoNT/C1, BoNT/D, BoNT/E, BoNT/F, BoNT/G, BoNT/X, or TeNT L-chain and/or H_{N} domain as described above.

The present invention also embraces clostridial neurotoxins that have a non-native protease cleavage site. In such clostridial neurotoxins, the native protease cleavage site (also known as the activation site, as described above) is modified or replaced with a protease cleavage site that is not native to that clostridial neurotoxin (i.e. an exogenous cleavage site). Such a site will require an exogenous protease for cleavage, which allows for improved control over the timing and location of cleavage events. Non-native protease cleavage sites that may be employed in clostridial neurotoxins include:

| | |
|---|---|
| TEV(Tobacco Etch | (ENLYFQ↓G)(SEQ ID NO: 122) |
| virus) | |
| Thrombin | (LVPR↓GS)(SEQ ID NO: 123) |
| PreScission | (LEVLFQ↓GP)(SEQ ID NO: 124). |
| Enterokinase | (DDDDK↓, SEQ ID NO: 125) |
| Factor Xa | (IEGR↓/IDGR↓, SEQ ID NOs: |
| | 126 and 127) |

Additional protease cleavage sites include recognition sequences that are cleaved by a non-cytotoxic protease, for example by the light chain of a clostridial neurotoxin. These include the SNARE (e.g. SNAP-25, syntaxin, VAMP) protein recognition sequences that are cleaved by non-cytotoxic proteases such as the light chain of a clostridial neurotoxin. Clostridial neurotoxins comprising non-native protease cleavage sites are described in U.S. Pat. No. 7,132,259, EP 1206554-B2 and US 2007/0166332, all of which are hereby incorporated by reference in their entirety. Also embraced by the term protease cleavage site is an intein, which is a self-cleaving sequence. The self-splicing reaction is controllable, for example by varying the concentration of reducing agent present.

Alternatively/additionally, a modified clostridial neurotoxin may comprise an (exogenous) activation loop as described in WO 2020/065336 A1.

The present invention also embraces clostridial neurotoxins comprising a “destructive cleavage site”. In said clostridial neurotoxins, a non-native protease cleavage site is incorporated into the clostridial neurotoxin, at a location chosen such that cleavage at said site will decrease the activity of, or inactivate, the clostridial neurotoxin. The destructive protease cleavage site can be susceptible to cleavage by a local protease, in the event that the clostridial neurotoxin, following administration, migrates to a non-target location. Suitable non-native protease cleavage sites include those described above. Clostridial neurotoxins comprising a destructive cleavage site are described in WO 2010/094905 and WO 2002/044199, both of which are hereby incorporated by reference in their entirety.

The modified clostridial neurotoxins of the present invention, especially the light chain component thereof, may be PEGylated—this may help to increase stability, for example duration of action of the light chain component. PEGylation is particularly preferred when the light chain comprises a BoNT/A, B or C1 protease. PEGylation preferably includes the addition of PEG to the N-terminus of the light chain component. By way of example, the N-terminus of a light chain may be extended with one or more amino acid (e.g. cysteine) residues, which may be the same or different. One or more of said amino acid residues may have its own PEG molecule attached (e.g. covalently attached) thereto. An example of this technology is described in WO2007/104567, which is hereby incorporated by reference in its entirety.

A modified clostridial neurotoxin of the present invention suitably finds utility in medicine or in cosmetics. In use, the modified clostridial neurotoxin is preferably in a di-chain form. Thus, in one aspect, the invention provides a modified clostridial neurotoxin according to the invention or a di-chain modified clostridial neurotoxin according to the invention for use in medicine. Similarly, the invention is directed to a method of treatment comprising administering a modified clostridial neurotoxin or di-chain modified clostridial neurotoxin of the invention to a subject in need thereof.

A “subject” as used herein may be a mammal, such as a human or other mammal. Preferably “subject” means a human subject.

The term “disorder” as used herein also encompasses a “disease”. In one embodiment the disorder is a disease.

The term “treat” or “treating” as used herein encompasses prophylactic treatment (e.g. to prevent onset of a disorder) as well as corrective treatment (treatment of a subject already suffering from a disorder). Preferably “treat” or “treating” as used herein means corrective treatment. The term “treat” or “treating” as used herein refers to the disorder and/or a symptom thereof.

Therefore a polypeptide of the invention may be administered to a subject in a therapeutically effective amount or a prophylactically effective amount. Preferably a modified clostridial neurotoxin of the invention is administered to a subject in a therapeutically effective amount.

A “therapeutically effective amount” is any amount of the modified clostridial neurotoxin, which when administered alone or in combination to a subject for treating said disorder (or a symptom thereof) is sufficient to effect such treatment of the disorder, or symptom thereof.

A “prophylactically effective amount” is any amount of the modified clostridial neurotoxin that, when administered alone or in combination to a subject inhibits or delays the onset or reoccurrence of a disorder (or a symptom thereof). In some embodiments, the prophylactically effective amount prevents the onset or reoccurrence of a disorder entirely. “Inhibiting” the onset means either lessening the likelihood of a disorder's onset (or symptom thereof), or preventing the onset entirely.

The dosage ranges for administration of the modified clostridial neurotoxins of the present invention are those to produce the desired therapeutic, cosmetic or prophylactic effect. It will be appreciated that the dosage range required depends on the precise nature of the modified clostridial neurotoxin or composition, the route of administration, the nature of the formulation, the age of the patient, the nature, extent or severity of the patient's condition, contraindications, if any, and the judgement of the attending physician. Variations in these dosage levels can be adjusted using standard empirical routines for optimisation.

Suitable daily dosages (per kg weight of patient) are in the range 0.0001-1 ng/kg, preferably 0.0001-0.5 ng/kg, more preferably 0.002-0.5 ng/kg, and particularly preferably 0.004-0.5 ng/kg.

The unit dosage can vary from less than 1 picogram to 30 ng, but typically will be in the region of 0.01 to 1 ng per dose, which may be administered daily or preferably less frequently, such as weekly or six monthly.

A particularly preferred dosing regimen is based on 0.05 ng of modified clostridial neurotoxin as the 1× dose. In this regard, preferred dosages are in the range 1×-100× (i.e. 0.05-5 ng).

In one aspect, the present invention provides a modified clostridial neurotoxin or di-chain modified clostridial neurotoxin as described above, for use in treating a disorder selected from: a condition associated with unwanted immune secretion, strabismus, blepharospasm, squint, dystonia (e.g. spasmodic dystonia, oromandibular dystonia, focal dystonia, tardive dystonia, laryngeal dystonia, limb dystonia, cervical dystonia), torticollis (e.g. spasmodic torticollis), beauty therapy (cosmetic) applications benefiting from cell/muscle incapacitation (via SNARE down-regulation or inactivation), neuromuscular disorder or condition of ocular motility (e.g. concomitant strabismus, vertical strabismus, lateral rectus palsy, nystagmus, dysthyroid myopathy), writer's cramp, bruxism, Wilson's disease, tremor, tics, segmental myoclonus, spasms, spasticity due to chronic multiple sclerosis, spasticity resulting in abnormal bladder control, animus, back spasm, charley horse, tension headaches, levator pelvic syndrome, spina bifida, tardive dyskinesia, Parkinson's disease, stuttering, hemifacial spasm, eyelid disorder, cerebral palsy, focal spasticity, spasmodic colitis, neurogenic bladder, anismus, limb spasticity, anal fissure, achalasia, dysphagia, lacrimation, hyperhydrosis, excessive salivation, excessive gastrointestinal secretions, muscle pain (e.g. pain from muscle spasms), headache pain (e.g. tension headache), brow furrows, skin wrinkles, cancer, uterine disorders, uro-genital disorders, urogenital-neurological disorders, chronic neurogenic inflammation, and a smooth muscle disorder.

In a related aspect, the present invention provides a method of treating a disorder, the method comprising administering a modified clostridial neurotoxin or di-chain modified clostridial neurotoxin as described above to a subject, wherein the disorder is selected from: a condition associated with unwanted immune secretion, strabismus, blepharospasm, squint, dystonia (e.g. spasmodic dystonia, oromandibular dystonia, focal dystonia, tardive dystonia, laryngeal dystonia, limb dystonia, cervical dystonia), torticollis (e.g. spasmodic torticollis), beauty therapy (cosmetic) applications benefiting from cell/muscle incapacitation (via SNARE down-regulation or inactivation), neuromuscular disorder or condition of ocular motility (e.g. concomitant strabismus, vertical strabismus, lateral rectus palsy, nystagmus, dysthyroid myopathy), writer's cramp, bruxism, Wilson's disease, tremor, tics, segmental myoclonus, spasms, spasticity due to chronic multiple sclerosis, spasticity resulting in abnormal bladder control, animus, back spasm, charley horse, tension headaches, levator pelvic syndrome, spina bifida, tardive dyskinesia, Parkinson's disease, stuttering, hemifacial spasm, eyelid disorder, cerebral palsy, focal spasticity, spasmodic colitis, neurogenic bladder, anismus, limb spasticity, anal fissure, achalasia, dysphagia, lacrimation, hyperhydrosis, excessive salivation, excessive gastrointestinal secretions, muscle pain (e.g. pain from muscle spasms), headache pain (e.g. tension headache), brow furrows, skin wrinkles, cancer, uterine disorders, uro-genital disorders, urogenital-neurological disorders, chronic neurogenic inflammation, and a smooth muscle disorder.

In a related aspect, the present invention provides use of a modified clostridial neurotoxin or di-chain modified clostridial neurotoxin as described above in the manufacture of a medicament for treating a disorder selected from: a condition associated with unwanted immune secretion, strabismus, blepharospasm, squint, dystonia (e.g. spasmodic dystonia, oromandibular dystonia, focal dystonia, tardive dystonia, laryngeal dystonia, limb dystonia, cervical dystonia), torticollis (e.g. spasmodic torticollis), beauty therapy (cosmetic) applications benefiting from cell/muscle incapacitation (via SNARE down-regulation or inactivation), neuromuscular disorder or condition of ocular motility (e.g. concomitant strabismus, vertical strabismus, lateral rectus palsy, nystagmus, dysthyroid myopathy), writer's cramp, bruxism, Wilson's disease, tremor, tics, segmental myoclonus, spasms, spasticity due to chronic multiple sclerosis, spasticity resulting in abnormal bladder control, animus, back spasm, charley horse, tension headaches, levator pelvic syndrome, spina bifida, tardive dyskinesia, Parkinson's disease, stuttering, hemifacial spasm, eyelid disorder, cerebral palsy, focal spasticity, spasmodic colitis, neurogenic bladder, anismus, limb spasticity, anal fissure, achalasia, dysphagia, lacrimation, hyperhydrosis, excessive salivation, excessive gastrointestinal secretions, muscle pain (e.g. pain from muscle spasms), headache pain (e.g. tension headache), brow furrows, skin wrinkles, cancer, uterine disorders, uro-genital disorders, urogenital-neurological disorders, chronic neurogenic inflammation, and a smooth muscle disorder.

In one aspect the invention provides a method of cosmetic treatment, the method comprising administering a modified clostridial neurotoxin or di-chain modified clostridial neurotoxin according to the invention to a subject. The method may be directed to a beauty therapy application benefiting from cell/muscle incapacitation (via SNARE down-regulation or inactivation). Said cosmetic treatment may treat: facial lines, glabellar lines, brow furrows, skin wrinkles, intrathecal lines, forehead lines, “bunny” lines, smile irregularities, chin irregularities, platysmal bands, “marionette” lines, lip lines, crow's feet, eyebrow irregularities, frown lines, worry lines, stretch marks, wounds, accidents, bites, surgery, and/or contour deficiencies of such areas as eyes, cheeks, nose, lips, forehead, and/or neck.

In one aspect, the invention provides a pharmaceutical composition comprising a modified clostridial neurotoxin or a di-chain modified clostridial neurotoxin of the invention and a pharmaceutically acceptable carrier, excipient, adjuvant, propellant and/or salt. A modified clostridial neurotoxin may be part of a pharmaceutical composition when employed in a therapeutic or cosmetic application described herein.

The modified clostridial neurotoxins of the present invention may be formulated for oral, parenteral, continuous infusion, inhalation or topical application. Compositions suitable for injection may be in the form of solutions, suspensions or emulsions, or dry powders which are dissolved or suspended in a suitable vehicle prior to use.

In the case of a modified clostridial neurotoxin that is to be delivered locally, the modified clostridial neurotoxin may be formulated as a cream (e.g. for topical application), or for sub-dermal injection.

Local delivery means may include an aerosol, or other spray (e.g. a nebuliser). In this regard, an aerosol formulation of a modified clostridial neurotoxin enables delivery to the lungs and/or other nasal and/or bronchial or airway passages.

Modified clostridial neurotoxins of the invention may be administered to a patient by intrathecal or epidural injection in the spinal column at the level of the spinal segment involved in the innervation of an affected organ.

A preferred route of administration is via laproscopic and/or localised, particularly intramuscular, injection.

Fluid dosage forms are typically prepared utilising the modified clostridial neurotoxin and a pyrogen-free sterile vehicle. The modified clostridial neurotoxin, depending on the vehicle and concentration used, can be either dissolved or suspended in the vehicle. In preparing solutions the modified clostridial neurotoxin can be dissolved in the vehicle, the solution being made isotonic if necessary by addition of sodium chloride and sterilised by filtration through a sterile filter using aseptic techniques before filling into suitable sterile vials or ampoules and sealing.

Alternatively, if solution stability is adequate, the solution in its sealed containers may be sterilised by autoclaving. Advantageously additives such as buffering, solubilising, stabilising, preservative or bactericidal, suspending or emulsifying agents and or local anaesthetic agents may be dissolved in the vehicle.

Dry powders, which are dissolved or suspended in a suitable vehicle prior to use, may be prepared by filling pre-sterilised ingredients into a sterile container using aseptic technique in a sterile area. Alternatively the ingredients may be dissolved into suitable containers using aseptic technique in a sterile area. The product is then freeze dried and the containers are sealed aseptically.

Parenteral suspensions, suitable for intramuscular, subcutaneous or intradermal injection, are prepared in substantially the same manner, except that the sterile components are suspended in the sterile vehicle, instead of being dissolved and sterilisation cannot be accomplished by filtration. The components may be isolated in a sterile state or alternatively it may be sterilised after isolation, e.g. by gamma irradiation.

Advantageously, a suspending agent for example polyvinylpyrrolidone is included in the composition(s) to facilitate uniform distribution of the components.

Administration in accordance with the present invention may take advantage of a variety of delivery technologies including microparticle encapsulation, viral delivery systems or high-pressure aerosol impingement.

The invention also provides screening methods for selecting clostridial neurotoxins that exhibit resistance to oxidation. Thus, in one aspect, there is provided a method for selecting an oxidation resistant clostridial neurotoxin, the method comprising:
(a) identifying an oxidizable amino acid residue of a clostridial neurotoxin heavy chain or portion thereof;
(b) producing a modified clostridial neurotoxin in which said oxidizable amino acid residue has been modified;
(c) subjecting the modified clostridial neurotoxin to oxidizing conditions;
(d) determining an activity level of the modified clostridial neurotoxin;
(e) comparing the activity level determined in step (d) with the activity level of an otherwise identical clostridial neurotoxin lacking the modification, wherein the otherwise identical clostridial neurotoxin lacking the modification has been subjected to oxidizing conditions; and
(f) selecting the modified clostridial neurotoxin when the activity level is higher than the activity level of the otherwise identical clostridial neurotoxin lacking the modification; or
(g) discarding/not selecting the modified clostridial neurotoxin when the activity level is the same or lower than the activity level of the otherwise identical clostridial neurotoxin lacking the modification.

The portion of the clostridial neurotoxin heavy chain may comprise (preferably consist of) a translocation domain (H_{N} domain) or portion thereof or a receptor binding domain (Hc domain) or a portion thereof. A portion of a receptor binding domain may comprise (preferably consist of) a translocation facilitating domain (H_{CN} domain) or a C-terminal portion of the He domain (H_{cc} domain), preferably an H_{cc} domain.

Step (a) of the method may be carried out in silico by analysing the nature of one or more amino acids present in the heavy chain or portion thereof and their susceptibility to oxidation. Such analysis may include determining the extent of surface exposure of the one or more amino acids. Amino acids that are susceptible to oxidation may include methionine, cysteine, histidine, tryptophan, tyrosine, and phenylalanine. Thus, one or more of methionine, cysteine, histidine, tryptophan, tyrosine, and phenylalanine may be identified in step (a) of the method as an oxidizable amino acid.

The modification is suitably any modification described herein, such as a substitution, insertion, deletion or indel, preferably a substitution. Preferably, a modification is substitution with an amino acid that is more resistant to oxidation than the amino acid identified. The modification may be substitution with: valine, leucine, glycine, threonine, alanine, isoleucine, aspartic acid, glutamic acid, arginine, lysine, asparagine, glutamine, serine, or proline. In one embodiment the modification may be substitution with: valine, leucine, or glycine. Preferably, substitution with valine or leucine. Alternatively, the modification may be substitution with a non-natural or non-standard amino acid, such as norleucine, isovaline, α-methyl valine, cycloleucine, or allo-threonine.

The invention also provides a modified clostridial neurotoxin selected by a method of the invention, preferably wherein the modified clostridial neurotoxin is oxidation resistant (e.g. is more resistant than an otherwise identical clostridial neurotoxin lacking the modification).

The skilled person will appreciate that the method for selecting an oxidation resistant clostridial neurotoxin may utilise any clostridial neurotoxin, as described herein. For example, the clostridial neurotoxin may be a botulinum neurotoxin or tetanus neurotoxin selected from BoNT/A (such as BoNT/A1, BoNT/A2, BoNT/A3, BoNT/A4, BoNT/A5, BoNT/A6, BoNT/A7 or BoNT/A8), BoNT/B, BoNT/C1, BoNT/D, BoNT/E, BoNT/F, BoNT/G, BoNT/X, and TeNT. For example, a clostridial neurotoxin employed in said method may comprise a polypeptide sequence having at least 70% sequence identity to any one of SEQ ID NOs: 2, 11, 20, 29, 38, 46, 54 or 128-135. In one embodiment, a clostridial neurotoxin employed in said method may comprise a polypeptide sequence having at least 80%, 90%, 95%, or 98% sequence identity to any one of SEQ ID NOs: 2, 11, 20, 29, 38, 46, 54 or 128-135. Preferably, a clostridial neurotoxin employed in said method comprises (more preferably consists of) any one of SEQ ID NOs: 2, 11, 20, 29, 38, 46, 54 or 128-135.

A modified clostridial neurotoxin in which said oxidizable amino acid residue has been modified may be produced using any technique known in the art. For example, as described above, a nucleic acid encoding the (unmodified) clostridial neurotoxin may be modified using standard molecular cloning techniques, for example by site-directed mutagenesis. Alternatively, a modified gene sequence can be chemically synthesised. In one embodiment, a modified clostridial neurotoxin is produced by a method comprising:
(i) providing a first nucleic acid encoding a clostridial neurotoxin and modifying the first nucleic acid to introduce a modification at an oxidizable amino acid identified in step (a), thereby producing a second nucleic acid, wherein the modification increases oxidative resistance of the modified clostridial neurotoxin when compared to an otherwise identical clostridial neurotoxin lacking the modification; or
(ii) synthesizing a nucleic acid that encodes a modified clostridial neurotoxin that comprises a modification at an oxidizable amino acid identified in step (a), wherein the modification increases oxidative resistance of the modified clostridial neurotoxin when compared to an otherwise identical clostridial neurotoxin lacking the modification, thereby providing a synthesized nucleic acid; and
(iii) expressing the second nucleic acid or the synthesized nucleic acid, respectively, thereby producing the modified clostridial neurotoxin;
(iv) isolating the modified clostridial neurotoxin; and
(v) activating the modified clostridial neurotoxin by contacting the (single-chain) modified clostridial neurotoxin with a protease that cleaves the (single-chain) modified clostridial neurotoxin at a recognition site (cleavage site) located between the light chain and heavy chain, thereby converting the (single-chain) modified clostridial neurotoxin into a di-chain modified clostridial neurotoxin, wherein the light chain and heavy chain are joined together by a disulphide bond.

It is preferred that, when carrying out the screening methods, the clostridial neurotoxin is subjected to forced oxidation conditions, e.g. as described in the present Examples (see “Forced Oxidation Study”). Nevertheless, the specific oxidation technique employed is not essential so long as the modified clostridial neurotoxin and the otherwise identical clostridial neurotoxin lacking the modification are subjected to the same oxidation technique under the same conditions to ensure comparability.

Any suitable activity assay can be used to quantify clostridial neurotoxin activity, such as the DAS assay described herein. However, it is preferred that a cell-based activity assay is carried out as described in the present Examples. Nevertheless, the specific assay employed is not essential so long as the activity of the modified clostridial neurotoxin and the otherwise identical clostridial neurotoxin lacking the modification are determined using the same assay under the same conditions to ensure comparability.

The term “the activity level is higher” as used in the context of the method for selecting an oxidation resistant clostridial neurotoxin means that the activity level is statistically-significantly higher.

The term “the activity level is the same” as used in the context of the method for selecting an oxidation resistant clostridial neurotoxin means that the activity level is not statistically-significantly different or identical.

The term “the activity level is lower” as used in the context of the method for selecting an oxidation resistant clostridial neurotoxin means that the activity level is statistically-significantly lower.

Statistical significance can be determined using any suitable technique, such as one-way ANOVA.

In one embodiment, where an initial methionine amino acid residue or a corresponding initial codon is indicated in any of the following SEQ ID NOs herein, said residue/codon is optional.

Embodiments related to the various modified clostridial neurotoxin of the invention are intended to be applied equally to nucleic acids of the invention, methods, uses or pharmaceutical compositions, and vice versa.

### Sequence Homology

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van Walle et al., Align-M—A New Algorithm for Multiple Alignment of Highly Divergent Sequences, 20(9) Bioinformatics:1428-1435 (2004).

Thus, percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes); preferably this method is used to align a sequence with SEQ ID NO: 2 to define amino acid position numbering as described herein.

The “percent sequence identity” between two or more nucleic acid or amino acid sequences is a function of the number of identical positions shared by the sequences. Thus, % identity may be calculated as the number of identical nucleotides/amino acids divided by the total number of nucleotides/amino acids, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

ALIGNMENT SCORES FOR
DETERMINING SEQUENCE IDENTITY

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

The percent identity is then calculated as:

$$\frac{Total number of identical matches}{\begin{matrix}[ length of the longer sequence plus the number gaps introduced \\ into the longer sequence in order to align the two sequences ]\end{matrix}} \times 100$$

Substantially homologous polypeptides are characterized as having one or more amino acid substitutions, deletions or additions. These changes are preferably of a minor nature, that is conservative amino acid substitutions (see below) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

CONSERVATIVE AMINO ACID SUBSTITUTIONS

| | |
|---|---|
| Basic: | arginine |
| | lysine |
| | histidine |
| Acidic: | glutamic acid |
| | aspartic acid |
| Polar: | glutamine |
| | asparagine |
| Hydrophobic: | leucine |
| | isoleucine |
| | valine |
| Aromatic: | phenylalanine |
| | tryptophan |
| | tyrosine |
| Small: | glycine |
| | alanine |
| | serine |
| | threonine |
| | methionine |

In addition to the 20 standard amino acids, non-standard amino acids (such as 4-hydroxyproline, 6-N-methyl lysine, 2-aminoisobutyric acid, isovaline and α-methyl serine) may be substituted for amino acid residues of the polypeptides of the present invention. A limited number of non-conservative amino acids, amino acids that are not encoded by the genetic code, and unnatural amino acids may be substituted for polypeptide amino acid residues. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine-scanning mutagenesis (Cunningham and Wells, Science 244: 1081-5, 1989). Sites of biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, NewYork (1994), and Hale & Marham, THE HARPER COLLINS DICTIONARY OF BIOLOGY, Harper Perennial, NY (1991) provide the skilled person with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure.

Amino acids are referred to herein using the name of the amino acid, the three letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. Thus, for example, reference to “a clostridial neurotoxin” includes a plurality of such candidate agents and reference to “the clostridial neurotoxin” includes reference to one or more clostridial neurotoxins and equivalents thereof known to those skilled in the art, and so forth.

Where the term “comprises” or “comprising” is used herein, in one embodiment said term may be replaced by “consists essentially of” or “consisting essentially of”. In another embodiment said term may be replaced by “consists of” or “consisting of”.

The publications discussed herein are provided solely for their disclosure prior to the filing date of the present application. Nothing herein is to be construed as an admission that such publications constitute prior art to the claims appended hereto.

### BRIEF DESCRIPTION OF THE DRAWINGS

Embodiments of the invention will now be described, by way of example only, with reference to the following Figures and Examples.

FIG. 1 shows the % change in control BoNT/A activity over time (“Activity”) following oxidation with hydrogen peroxide, as well as the % change in the level of oxidation associated with the peptide comprising methionine residue 1144.

FIG. 2 shows the % change in control BoNT/A activity over time (“Activity”) following oxidation with hydrogen peroxide, as well as the % change in the level of oxidation associated with the peptide comprising methionine residue 1144.

FIG. 3 shows an SDS-PAGE gel of purified modified BoNT/A (M1144V) before Lys-C activation (−) and after Lys-C activation (+). Single-chain un-cleaved modified BoNT/A is shown together with the heavy chain (H-chain) and light chain (L-chain) components of di-chain modified BoNT/A. M=marker.

FIG. 4 shows the % change in modified BoNT/A activity over time (“Activity”) following oxidation with hydrogen peroxide, as well as the % change in the level of oxidation associated with the peptide comprising V1144.

FIG. 5 shows: (A) the SV2c binding ‘on’ rate for wild-type BoNT/A CLD1040 as well as BoNT/A M1144V and BoNT/A M1144L (One way ANOVA: P=0.0005 (***), P<0.0001 (****)); and (B) the SV2c binding ‘off’ rate forwild-type BoNT/A CLD1040 as well as BoNT/A M1144V and BoNT/A M1144L (One way ANOVA: P<0.0001 (****)).

### SEQUENCE LISTING

SEQ ID NO: 1—(BoNT/A1 Nucleic Acid Sequence)
SEQ ID NO: 2—(BoNT/A1 Polypeptide Sequence)
SEQ ID NO: 3—(Modified BoNT/A1 (M1144V) Polypeptide Sequence)
SEQ ID NO: 4—(Modified BoNT/A1 (M1144G) Polypeptide Sequence)
SEQ ID NO: 5—(Modified BoNT/A1 (M1144L) Polypeptide Sequence)
SEQ ID NO: 6—(Modified BoNT/A1 (M1144T) Polypeptide Sequence)
SEQ ID NO: 7—(Modified BoNT/A1 (M1144A) Polypeptide Sequence)
SEQ ID NO: 8—(Modified BoNT/A1 (M11441) Polypeptide Sequence)
SEQ ID NO: 9—(Modified BoNT/A1 (M1144 Deletion) Polypeptide Sequence)
SEQ ID NO: 10—(BoNT/A1 (N886K, N930K, S955K, Q991K, N1026K, N1052K, Q1229K) Nucleic Acid Sequence)
SEQ ID NO: 11—(BoNT/A1 (N886K, N930K, S955K, Q991K, N1026K, N1052K, Q1229K) Polypeptide Sequence)
SEQ ID NO: 12—(Modified BoNT/A1 (N886K, N930K, S955K, Q991K, N1026K, N1052K, Q1229K, M1144V) Polypeptide Sequence)
SEQ ID NO: 13—(Modified BoNT/A1 (N886K, N930K, S955K, Q991K, N1026K, N1052K, Q1229K, M1144G) Polypeptide Sequence)
SEQ ID NO: 14—(Modified BoNT/A1 (N886K, N930K, S955K, Q991K, N1026K, N1052K, Q1229K, M1144L) Polypeptide Sequence)
SEQ ID NO: 15—(Modified BoNT/A1 (N886K, N930K, S955K, Q991K, N1026K, N1052K, Q1229K, M1144T) Polypeptide Sequence)
SEQ ID NO: 16—(Modified BoNT/A1 (N886K, N930K, S955K, Q991K, N1026K, N1052K, Q1229K, M1144A) Polypeptide Sequence)
SEQ ID NO: 17—(Modified BoNT/A1 (N886K, N930K, S955K, Q991K, N1026K, N1052K, Q1229K, M11441) Polypeptide Sequence)
SEQ ID NO: 18—(Modified BoNT/A1 (N886K, N930K, S955K, Q991K, N1026K, N1052K, Q1229K, M1144 Deletion) Polypeptide Sequence)
SEQ ID NO: 19—(BoNT/A1 (N954K, N930K, S955K, Q991K, N1026K, N1052K, Q1229K) Nucleic Acid Sequence)
SEQ ID NO: 20—(BoNT/A1 (N954K, N930K, S955K, Q991K, N1026K, N1052K, Q1229K) Polypeptide Sequence)
SEQ ID NO: 21—(Modified BoNT/A1 (N954K, N930K, S955K, Q991K, N1026K, N1052K, Q1229K, M1144V) Polypeptide Sequence)
SEQ ID NO: 22—(Modified BoNT/A1 (N954K, N930K, S955K, Q991K, N1026K, N1052K, Q1229K, M1144G) Polypeptide Sequence)
SEQ ID NO: 23—(Modified BoNT/A1 (N954K, N930K, S955K, Q991K, N1026K, N1052K, Q1229K, M1144L) Polypeptide Sequence)
SEQ ID NO: 24—(Modified BoNT/A1 (N954K, N930K, S955K, Q991K, N1026K, N1052K, Q1229K, M1144T) Polypeptide Sequence)
SEQ ID NO: 25—(Modified BoNT/A1 (N954K, N930K, S955K, Q991K, N1026K, N1052K, Q1229K, M1144A) Polypeptide Sequence)
SEQ ID NO: 26—(Modified BoNT/A1 (N954K, N930K, S955K, Q991K, N1026K, N1052K, Q1229K, M11441) Polypeptide Sequence)
SEQ ID NO: 27—(Modified BoNT/A1 (N954K, N930K, S955K, Q991K, N1026K, N1052K, Q1229K, M1144 Deletion) Polypeptide Sequence)
SEQ ID NO: 28—(BoNT/A1 (N930K, S955K, Q991K, N1026K, N1052K, Q1229K, N1025K) Nucleic Acid Sequence)
SEQ ID NO: 29—(BoNT/A1 (N930K, S955K, Q991K, N1026K, N1052K, Q1229K, N1025K) Polypeptide Sequence)
SEQ ID NO: 30—(Modified BoNT/A1 (N930K, S955K, Q991K, N1026K, N1052K, Q1229K, N1025K, M1144V) Polypeptide Sequence)
SEQ ID NO: 31—(Modified BoNT/A1 (N930K, S955K, Q991K, N1026K, N1052K, Q1229K, N1025K, M1144G) Polypeptide Sequence)
SEQ ID NO: 32—(Modified BoNT/A1 (N930K, S955K, Q991K, N1026K, N1052K, Q1229K, N1025K, M1144L) Polypeptide Sequence)
SEQ ID NO: 33—(Modified BoNT/A1 (N930K, S955K, Q991K, N1026K, N1052K, Q1229K, N1025K, M1144T) Polypeptide Sequence)
SEQ ID NO: 34—(Modified BoNT/A1 (N930K, S955K, Q991K, N1026K, N1052K, Q1229K, N1025K, M1144A) Polypeptide Sequence)
SEQ ID NO: 35—(Modified BoNT/A1 (N930K, S955K, Q991K, N1026K, N1052K, Q1229K, N1025K, M11441) Polypeptide Sequence)
SEQ ID NO: 36—(Modified BoNT/A1 (N930K, S955K, Q991K, N1026K, N1052K, Q1229K, N1025K, M1144 Deletion) Polypeptide Sequence)
SEQ ID NO: 37—(BoNT/A1 (N1188R, D1213R, G1215R, N1216R, N1242R, N1243R, S1274R, T1277R) Nucleic Acid Sequence)
SEQ ID NO: 38—(BoNT/A1 (N1188R, D1213R, G1215R, N1216R, N1242R, N1243R, S1274R, T1277R) Polypeptide Sequence)
SEQ ID NO: 39—(Modified BoNT/A1 (N1188R, D1213R, G1215R, N1216R, N1242R, N1243R, S1274R, T1277R, M1144V) Polypeptide Sequence)
SEQ ID NO: 40—(Modified BoNT/A1 (N1188R, D1213R, G1215R, N1216R, N1242R, N1243R, S1274R, T1277R, M1144G) Polypeptide Sequence)
SEQ ID NO: 41—(Modified BoNT/A1 (N1188R, D1213R, G1215R, N1216R, N1242R, N1243R, S1274R, T1277R, M1144L) Polypeptide Sequence)
SEQ ID NO: 42—(Modified BoNT/A1 (N1188R, D1213R, G1215R, N1216R, N1242R, N1243R, S1274R, T1277R, M1144T) Polypeptide Sequence)
SEQ ID NO: 43—(Modified BoNT/A1 (N1188R, D1213R, G1215R, N1216R, N1242R, N1243R, S1274R, T1277R, M1144A) Polypeptide Sequence)
SEQ ID NO: 44—(Modified BoNT/A1 (N1188R, D1213R, G1215R, N1216R, N1242R, N1243R, S1274R, T1277R, M11441) Polypeptide Sequence)
SEQ ID NO: 45—(Modified BoNT/A1 (N1188R, D1213R, G1215R, N1216R, N1242R, N1243R, S1274R, T1277R, M1144 Deletion) Polypeptide Sequence)
SEQ ID NO: 46—(BoNT/A3 Polypeptide Sequence)
SEQ ID NO: 47—(Modified BoNT/A3 (M1140V) Polypeptide Sequence)
SEQ ID NO: 48—(Modified BoNT/A3 (M1140G) Polypeptide Sequence)
SEQ ID NO: 49—(Modified BoNT/A3 (M1140L) Polypeptide Sequence)
SEQ ID NO: 50—(Modified BoNT/A3 (M1140T) Polypeptide Sequence)
SEQ ID NO: 51—(Modified BoNT/A3 (M1140A) Polypeptide Sequence)
SEQ ID NO: 52—(Modified BoNT/A3 (M11401) Polypeptide Sequence)
SEQ ID NO: 53—(Modified BoNT/A3 (M1140 Deletion) Polypeptide Sequence)
SEQ ID NO: 54—(BoNT/A4 Polypeptide Sequence)
SEQ ID NO: 55—(Modified BoNT/A4 (M1144V) Polypeptide Sequence)
SEQ ID NO: 56—(Modified BoNT/A4 (M1144G) Polypeptide Sequence)
SEQ ID NO: 57—(Modified BoNT/A4 (M1144L) Polypeptide Sequence)
SEQ ID NO: 58—(Modified BoNT/A4 (M1144T) Polypeptide Sequence)
SEQ ID NO: 59—(Modified BoNT/A4 (M1144A) Polypeptide Sequence)
SEQ ID NO: 60—(Modified BoNT/A4 (M11441) Polypeptide Sequence)
SEQ ID NO: 61—(Modified BoNT/A4 (M1144 Deletion) Polypeptide Sequence)
SEQ ID NO: 62—(BoNT/A1 H_{cc} domain Polypeptide Sequence)
SEQ ID NO: 63—(Modified BoNT/A1 H_{cc} domain (M1144V) Polypeptide Sequence)
SEQ ID NO: 64—(Modified BoNT/A1 H_{cc} domain (M1144G) Polypeptide Sequence)
SEQ ID NO: 65—(Modified BoNT/A1 H_{cc} domain (M1144L) Polypeptide Sequence)
SEQ ID NO: 66—(Modified BoNT/A1 H_{cc} domain (M1144T) Polypeptide Sequence)
SEQ ID NO: 67—(Modified BoNT/A1 H_{cc} domain (M1144A) Polypeptide Sequence)
SEQ ID NO: 68—(Modified BoNT/A1 H_{cc} domain (M11441) Polypeptide Sequence)
SEQ ID NO: 69—(Modified BoNT/A1 H_{cc} domain (M1144 Deletion) Polypeptide Sequence)
SEQ ID NO: 70—(BoNT/A1 H_{cc} domain (Q1229K) Polypeptide Sequence)
SEQ ID NO: 71—(Modified BoNT/A1 H_{cc} domain (Q1229K, M1144V) Polypeptide Sequence)
SEQ ID NO: 72—(Modified BoNT/A1 H_{cc} domain (Q1229K, M1144G) Polypeptide Sequence)
SEQ ID NO: 73—(Modified BoNT/A1 H_{cc} domain (Q1229K, M1144L) Polypeptide Sequence)
SEQ ID NO: 74—(Modified BoNT/A1 H_{cc} domain (Q1229K, M1144T) Polypeptide Sequence)
SEQ ID NO: 75—(Modified BoNT/A1 H_{cc} domain (Q1229K, M1144A) Polypeptide Sequence)
SEQ ID NO: 76—(Modified BoNT/A1 H_{cc} domain (Q1229K, M11441) Polypeptide Sequence)
SEQ ID NO: 77—(Modified BoNT/A1 H_{cc} domain (Q1229K, M1144 Deletion) Polypeptide Sequence)
SEQ ID NO: 78—(BoNT/A1 H_{cc} domain (N1188R, D1213R, G1215R, N1216R, N1242R, N1243R, S1274R, T1277R) Polypeptide Sequence)
SEQ ID NO: 79—(Modified BoNT/A1 H_{cc} domain (N1188R, D1213R, G1215R, N1216R, N1242R, N1243R, S1274R, T1277R, M1144V) Polypeptide
SEQ ID NO: 80—(Modified BoNT/A1 H_{cc} domain (N1188R, D1213R, G1215R, N1216R, N1242R, N1243R, S1274R, T1277R, M1144G) Polypeptide Sequence)
SEQ ID NO: 81—(Modified BoNT/A1 H_{cc} domain (N1188R, D1213R, G1215R, N1216R, N1242R, N1243R, S1274R, T1277R, M1144L) Polypeptide Sequence)
SEQ ID NO: 82—(Modified BoNT/A1 H_{cc} domain (N1188R, D1213R, G1215R, N1216R, N1242R, N1243R, S1274R, T1277R, M1144T) Polypeptide Sequence)
SEQ ID NO: 83—(Modified BoNT/A1 H_{cc} domain (N1188R, D1213R, G1215R, N1216R, N1242R, N1243R, S1274R, T1277R, M1144A) Polypeptide Sequence)
SEQ ID NO: 84—(Modified BoNT/A1 H_{cc} domain (N1188R, D1213R, G1215R, N1216R, N1242R, N1243R, S1274R, T1277R, M11441) Polypeptide Sequence)
SEQ ID NO: 85—(Modified BoNT/A1 H_{cc} domain (N1188R, D1213R, G1215R, N1216R, N1242R, N1243R, S1274R, T1277R, M1144 Deletion) Polypeptide Sequence)
SEQ ID NO: 86—(BoNT/A3 H_{cc} domain Polypeptide Sequence)
SEQ ID NO: 87—(Modified BoNT/A3 H_{cc} domain (M1144V) Polypeptide Sequence)
SEQ ID NO: 88—(Modified BoNT/A3 H_{cc} domain (M1144G) Polypeptide Sequence)
SEQ ID NO: 89—(Modified BoNT/A3 H_{cc} domain (M1144L) Polypeptide Sequence)
SEQ ID NO: 90—(Modified BoNT/A3 H_{cc} domain (M1144T) Polypeptide Sequence)
SEQ ID NO: 91—(Modified BoNT/A3 H_{cc} domain (M1144A) Polypeptide Sequence)
SEQ ID NO: 92—(Modified BoNT/A3 H_{cc} domain (M11441) Polypeptide Sequence)
SEQ ID NO: 93—(Modified BoNT/A3 H_{cc} domain (M1144 Deletion) Polypeptide Sequence)
SEQ ID NO: 94—(BoNT/A4 H_{cc} domain Polypeptide Sequence)
SEQ ID NO: 95—(Modified BoNT/A4 H_{cc} domain (M1144V) Polypeptide Sequence)
SEQ ID NO: 96—(Modified BoNT/A4 H_{cc} domain (M1144G) Polypeptide Sequence)
SEQ ID NO: 97—(Modified BoNT/A4 H_{cc} domain (M1144L) Polypeptide Sequence)
SEQ ID NO: 98—(Modified BoNT/A4 H_{cc} domain (M1144T) Polypeptide Sequence)
SEQ ID NO: 99—(Modified BoNT/A4 H_{cc} domain (M1144A) Polypeptide Sequence)
SEQ ID NO: 100—(Modified BoNT/A4 H_{cc} domain (M11441) Polypeptide Sequence)
SEQ ID NO: 101—(Modified BoNT/A4 H_{cc} domain (M1144 Deletion) Polypeptide Sequence)
SEQ ID NO: 102—(Modified SV2 Binding Domain Consensus Sequence 1)
SEQ ID NO: 103—(BoNT/A1 Hall Str (Reference) SV2 Binding Domain)
SEQ ID NO: 104—(BoNT/A1 CDC297 SV2 Binding Domain)
SEQ ID NO: 105—(BoNT/A3 Loch Maree SV2 Binding Domain)
SEQ ID NO: 106—(BoNT/A4 SV2 Binding Domain)
SEQ ID NO: 107—(Modified SV2 Binding Domain Consensus Sequence 2)
SEQ ID NO: 108—(Modified SV2 Binding Domain Consensus Sequence 3)
SEQ ID NO: 109—(Modified SV2 Binding Domain Consensus Sequence 4)
SEQ ID NO: 110—(Modified SV2 Binding Domain Consensus Sequence 5)
SEQ ID NO: 111—(Modified SV2 Binding Domain Consensus Sequence 6)
SEQ ID NO: 112—(Modified SV2 Binding Domain A)
SEQ ID NO: 113—(Modified SV2 Binding Domain B)
SEQ ID NO: 114—(Modified SV2 Binding Domain C)
SEQ ID NO: 115—(Modified SV2 Binding Domain D)
SEQ ID NO: 116—(Influenza Virus Haemagglutinin)
SEQ ID NO: 117—(Leucine-Based Motif 1)
SEQ ID NO: 118—(Leucine-Based Motif 2)
SEQ ID NO: 119—(Leucine-Based Motif 3)
SEQ ID NO: 120—(Leucine-Based Motif 4)
SEQ ID NO: 121—(Tyrosine-based Motif)
SEQ ID NO: 122—(TEV Cleavage Site)
SEQ ID NO: 123—(Thrombin Cleavage Site)
SEQ ID NO: 124—(PreScission Cleavage Site)
SEQ ID NO: 125—(Enterokinase Cleavage Site)
SEQ ID NO: 126—(Factor Xa Cleavage Site 1)
SEQ ID NO: 127—(Factor Xa Cleavage Site 2)
SEQ ID NO: 128—(Polypeptide Sequence of BoNT/B—UniProt P10844)
SEQ ID NO: 129—(Polypeptide Sequence of BoNT/C—UniProt P18640)
SEQ ID NO: 130—(Polypeptide Sequence of BoNT/D—UniProt P19321)
SEQ ID NO: 131—(Polypeptide Sequence of BoNT/E—UniProt Q00496)
SEQ ID NO: 132—(Polypeptide Sequence of BoNT/F—UniProt A7GBG3)
SEQ ID NO: 133—(Polypeptide Sequence of BoNT/G—UniProt Q60393)
SEQ ID NO: 134—(Polypeptide Sequence of TeNT—UniProt P04958)
SEQ ID NO: 135—(Polypeptide Sequence of BoNT/X)
SEQ ID NO: 136—(Modified BoNT/A1 (M1144V) Nucleic Acid Sequence)
SEQ ID NO: 137—(Modified BoNT/A1 (M1144G) Nucleic Acid Sequence)
SEQ ID NO: 138—(Modified BoNT/A1 (M1144L) Nucleic Acid Sequence)
SEQ ID NO: 139—(Polypeptide Sequence of BoNT/A2—UniProt D3|V23)
SEQ ID NO: 140—(Polypeptide Sequence of BoNT/A5 v.1—UniProt C7BEA8)
SEQ ID NO: 141—(Polypeptide Sequence of BoNT/A5 v.2—UniProt C11PK2)
SEQ ID NO: 142—(Polypeptide Sequence of BoNT/A6—ACW83608.1, Accession #FJ981696)
SEQ ID NO: 143—(Polypeptide Sequence of BoNT/A7—GenBank: AFV13854.1, Accession #JQ954969.1)
SEQ ID NO: 144—(Polypeptide Sequence of BoNT/A8—GenBank: AJA05787.1, Accession #KM233166)

SEQ ID NO: 1
(BoNT/A1 Nucleic Acid Sequence)
ATGCCATTCGTCAACAAGCAATTCAACTACAAAGACCCAGTCAACGGCGTCGACATCGCATACATCAAGATTCCG
AACGCCGGTCAAATGCAGCCGGTTAAGGCTTTTAAGATCCACAACAAGATTTGGGTTATCCCGGAGCGTGACACC
TTCACGAACCCGGAAGAAGGCGATCTGAACCCGCCACCGGAAGCGAAGCAAGTCCCTGTCAGCTACTACGATTCG
ACGTACCTGAGCACGGATAACGAAAAAGATAACTACCTGAAAGGTGTGACCAAGCTGTTCGAACGTATCTACAGC
ACGGATCTGGGTCGCATGCTGCTGACTAGCATTGTTCGCGGTATCCCGTTCTGGGGTGGTAGCACGATTGACACC
GAACTGAAGGTTATCGACACTAACTGCATTAACGTTATTCAACCGGATGGTAGCTATCGTAGCGAAGAGCTGAAT
CTGGTCATCATTGGCCCGAGCGCAGACATTATCCAATTCGAGTGCAAGAGCTTTGGTCACGAGGTTCTGAATCTG
ACCCGCAATGGCTATGGTAGCACCCAGTACATTCGTTTTTCGCCGGATTTTACCTTCGGCTTTGAAGAGAGCCTG
GAGGTTGATACCAATCCGTTGCTGGGTGCGGGCAAATTCGCTACCGATCCGGCTGTCACGCTGGCCCATGAACTG
ATCCACGCAGGCCACCGCCTGTACGGCATTGCCATCAACCCAAACCGTGTGTTCAAGGTTAATACGAATGCATAC
TACGAGATGAGCGGCCTGGAAGTCAGCTTCGAAGAACTGCGCACCTTCGGTGGCCATGACGCTAAATTCATTGAC
AGCTTGCAAGAGAATGAGTTCCGTCTGTACTACTATAACAAATTCAAAGACATTGCAAGCACGTTGAACAAGGCC
AAAAGCATCGTTGGTACTACCGCGTCGTTGCAGTATATGAAGAATGTGTTTAAAGAGAAGTACCTGCTGTCCGAG
GATACCTCCGGCAAGTTTAGCGTTGATAAGCTGAAGTTTGACAAACTGTACAAGATGCTGACCGAGATTTACACC
GAGGACAACTTTGTGAAATTCTTCAAAGTGTTGAATCGTAAAACCTATCTGAATTTTGACAAAGCGGTTTTCAAG
ATTAACATCGTGCCGAAGGTGAACTACACCATCTATGACGGTTTTAACCTGCGTAACACCAACCTGGCGGCGAAC
TTTAACGGTCAGAATACGGAAATCAACAACATGAATTTCACGAAGTTGAAGAACTTCACGGGTCTGTTCGAGTTC
TATAAGCTGCTGTGCGTGCGCGGTATCATCACCAGCAAAACCAAAAGCCTGGACAAAGGCTACAACAAGGCGCTG
AATGACCTGTGCATTAAGGTAAACAATTGGGATCTGTTCTTTTCGCCATCCGAAGATAATTTTACCAACGACCTG
AACAAGGGTGAAGAAATCACCAGCGATACGAATATTGAAGCAGCGGAAGAGAATATCAGCCTGGATCTGATCCAG
CAGTACTATCTGACCTTTAACTTCGACAATGAACCGGAGAACATTAGCATTGAGAATCTGAGCAGCGACATTATC
GGTCAGCTGGAACTGATGCCGAATATCGAACGTTTCCCGAACGGCAAAAAGTACGAGCTGGACAAGTACACTATG
TTCCATTACCTGCGTGCACAGGAGTTTGAACACGGTAAAAGCCGTATCGCGCTGACCAACAGCGTTAACGAGGCC
CTGCTGAACCCGAGCCGTGTCTATACCTTCTTCAGCAGCGACTATGTTAAGAAAGTGAACAAAGCCACTGAGGCC
GCGATGTTCCTGGGCTGGGTGGAACAGCTGGTATATGACTTCACGGACGAGACGAGCGAAGTGAGCACTACCGAC
AAAATTGCTGATATTACCATCATTATCCCGTATATTGGTCCGGCACTGAACATTGGCAACATGCTGTACAAAGAC
GATTTTGTGGGTGCCCTGATCTTCTCCGGTGCCGTGATTCTGCTGGAGTTCATTCCGGAGATTGCGATCCCGGTG
TTGGGTACCTTCGCGCTGGTGTCCTACATCGCGAATAAGGTTCTGACGGTTCAGACCATCGATAACGCGCTGTCG
AAACGTAATGAAAAATGGGACGAGGTTTACAAATACATTGTTACGAATTGGCTGGCGAAAGTCAATACCCAGATC
GACCTGATCCGTAAGAAAATGAAAGAGGCGCTGGAGAATCAGGCGGAGGCCACCAAAGCAATTATCAACTACCAA
TACAACCAGTACACGGAAGAAGAGAAGAATAACATTAACTTCAATATCGATGATTTGAGCAGCAAGCTGAATGAA
TCTATCAACAAAGCGATGATCAATATCAACAAGTTTTTGAATCAGTGTAGCGTTTCGTACCTGATGAATAGCATG
ATTCCGTATGGCGTCAAACGTCTGGAGGACTTCGACGCCAGCCTGAAAGATGCGTTGCTGAAATACATTTACGAC
AATCGTGGTACGCTGATTGGCCAAGTTGACCGCTTGAAAGACAAAGTTAACAATACCCTGAGCACCGACATCCCA
TTTCAACTGAGCAAGTATGTTGATAATCAACGTCTGTTGAGCACTTTCACCGAGTATATCAAAAACATCATCAAT
ACTAGCATTCTGAACCTGCGTTACGAGAGCAATCATCTGATTGATCTGAGCCGTTATGCAAGCAAGATCAACATC
GGTAGCAAGGTCAATTTTGACCCGATCGATAAGAACCAGATCCAGCTGTTTAATCTGGAATCGAGCAAAATTGAG
GTTATCCTGAAAAACGCCATTGTCTACAACTCCATGTACGAGAATTTCTCCACCAGCTTCTGGATTCGCATCCCG
AAATACTTCAACAGCATTAGCCTGAACAACGAGTATACTATCATCAACTGTATGGAGAACAACAGCGGTTGGAAG
GTGTCTCTGAACTATGGTGAGATCATTTGGACCTTGCAGGACACCCAAGAGATCAAGCAGCGCGTCGTGTTCAAG
TACTCTCAAATGATCAACATTTCCGATTACATTAATCGTTGGATCTTCGTGACCATTACGAATAACCGTCTGAAT
AACAGCAAGATTTACATCAATGGTCGCTTGATCGATCAGAAACCGATTAGCAACCTGGGTAATATCCACGCAAGC
AACAACATTATGTTCAAATTGGACGGTTGCCGCGATACCCATCGTTATATCTGGATCAAGTATTTCAACCTGTTT
GATAAAGAACTGAATGAGAAGGAGATCAAAGATTTGTATGACAACCAATCTAACAGCGGCATTTTGAAGGACTTC
TGGGGCGATTATCTGCAATACGATAAGCCGTACTATATGCTGAACCTGTATGATCCGAACAAATATGTGGATGTC
AATAATGTGGGTATTCGTGGTTACATGTATTTGAAGGGTCCGCGTGGCAGCGTTATGACGACCAACATTTACCTG
AACTCTAGCCTGTACCGTGGTACGAAATTCATCATTAAGAAATATGCCAGCGGCAACAAAGATAACATTGTGCGT
AATAACGATCGTGTCTACATCAACGTGGTCGTGAAGAATAAAGAGTACCGTCTGGCGACCAACGCTTCGCAGGCG
GGTGTTGAGAAAATTCTGAGCGCGTTGGAGATCCCTGATGTCGGTAATCTGAGCCAAGTCGTGGTTATGAAGAGC
AAGAACGACCAGGGTATCACTAACAAGTGCAAGATGAACCTGCAAGACAACAATGGTAACGACATCGGCTTTATT
GGTTTCCACCAGTTCAACAATATTGCTAAACTGGTAGCGAGCAATTGGTACAATCGTCAGATTGAGCGCAGCAGC
CGTACTTTGGGCTGTAGCTGGGAGTTTATCCCGGTCGATGATGGTTGGGGCGAACGTCCGCTG
SEQ ID NO: 2
(BoNT/A1 Polypeptide Sequence)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESNHLIDLSRYASKINI
GSKVNFDPIDKNQIQLFNLESSKIEVILKNAIVYNSMYENFSTSFWIRIPKYFNSISLNNEYTIINCMENNSGWK
VSLNYGEIIWTLQDTQEIKQRVVFKYSQMINISDYINRWIFVTITNNRLNNSKIYINGRLIDQKPISNLGNIHAS
NNIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVMTTNIYLNSSLYRGTKFIIKKYASGNKDNIVRNNDRVYINVVVKNKEYRLATNASQA
GVEKILSALEIPDVGNLSQVVVMKSKNDQGITNKCKMNLQDNNGNDIGFIGFHQFNNIAKLVASNWYNRQIERSS
RTLGCSWEFIPVDDGWGERPL
SEQ ID NO: 3
(Modified BoNT/A1 (M1144V) Polypeptide Sequence)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESNHLIDLSRYASKINI
GSKVNFDPIDKNQIQLFNLESSKIEVILKNAIVYNSMYENFSTSFWIRIPKYFNSISLNNEYTIINCMENNSGWK
VSLNYGEIIWTLQDTQEIKQRVVFKYSQMINISDYINRWIFVTITNNRLNNSKIYINGRLIDQKPISNLGNIHAS
NNIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVVTTNIYLNSSLYRGTKFIIKKYASGNKDNIVRNNDRVYINVVVKNKEYRLATNASQA
GVEKILSALEIPDVGNLSQVVVMKSKNDQGITNKCKMNLQDNNGNDIGFIGFHQFNNIAKLVASNWYNRQIERSS
RTLGCSWEFIPVDDGWGERPL
SEQ ID NO: 4
(Modified BoNT/A1 (M1144G) Polypeptide Sequence)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESNHLIDLSRYASKINI
GSKVNFDPIDKNQIQLFNLESSKIEVILKNAIVYNSMYENFSTSFWIRIPKYFNSISLNNEYTIINCMENNSGWK
VSLNYGEIIWTLQDTQEIKQRVVFKYSQMINISDYINRWIFVTITNNRLNNSKIYINGRLIDQKPISNLGNIHAS
NNIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVGTTNIYLNSSLYRGTKFIIKKYASGNKDNIVRNNDRVYINVVVKNKEYRLATNASQA
GVEKILSALEIPDVGNLSQVVVMKSKNDQGITNKCKMNLQDNNGNDIGFIGFHQFNNIAKLVASNWYNRQIERSS
RTLGCSWEFIPVDDGWGERPL
SEQ ID NO: 5
(Modified BoNT/A1 (M1144L) Polypeptide Sequence)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESNHLIDLSRYASKINI
GSKVNFDPIDKNQIQLFNLESSKIEVILKNAIVYNSMYENFSTSFWIRIPKYFNSISLNNEYTIINCMENNSGWK
VSLNYGEIIWTLQDTQEIKQRVVFKYSQMINISDYINRWIFVTITNNRLNNSKIYINGRLIDQKPISNLGNIHAS
NNIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVLTTNIYLNSSLYRGTKFIIKKYASGNKDNIVRNNDRVYINVVVKNKEYRLATNASQA
GVEKILSALEIPDVGNLSQVVVMKSKNDQGITNKCKMNLQDNNGNDIGFIGFHQFNNIAKLVASNWYNRQIERSS
RTLGCSWEFIPVDDGWGERPL
SEQ ID NO: 6
(Modified BoNT/A1 (M1144T) Polypeptide Sequence)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESNHLIDLSRYASKINI
GSKVNFDPIDKNQIQLFNLESSKIEVILKNAIVYNSMYENFSTSFWIRIPKYFNSISLNNEYTIINCMENNSGWK
VSLNYGEIIWTLQDTQEIKQRVVFKYSQMINISDYINRWIFVTITNNRLNNSKIYINGRLIDQKPISNLGNIHAS
NNIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVTTTNIYLNSSLYRGTKFIIKKYASGNKDNIVRNNDRVYINVVVKNKEYRLATNASQA
GVEKILSALEIPDVGNLSQVVVMKSKNDQGITNKCKMNLQDNNGNDIGFIGFHQFNNIAKLVASNWYNRQIERSS
RTLGCSWEFIPVDDGWGERPL
SEQ ID NO: 7 (Modified BoNT/A1 (M1144A) Polypeptide Sequence)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESNHLIDLSRYASKINI
GSKVNFDPIDKNQIQLFNLESSKIEVILKNAIVYNSMYENFSTSFWIRIPKYFNSISLNNEYTIINCMENNSGWK
VSLNYGEIIWTLQDTQEIKQRVVFKYSQMINISDYINRWIFVTITNNRLNNSKIYINGRLIDQKPISNLGNIHAS
NNIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVATTNIYLNSSLYRGTKFIIKKYASGNKDNIVRNNDRVYINVVVKNKEYRLATNASQA
GVEKILSALEIPDVGNLSQVVVMKSKNDQGITNKCKMNLQDNNGNDIGFIGFHQFNNIAKLVASNWYNRQIERSS
RTLGCSWEFIPVDDGWGERPL
SEQ ID NO: 8
(Modified BoNT/A1 (M11441) Polypeptide Sequence)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESNHLIDLSRYASKINI
GSKVNFDPIDKNQIQLFNLESSKIEVILKNAIVYNSMYENFSTSFWIRIPKYFNSISLNNEYTIINCMENNSGWK
VSLNYGEIIWTLQDTQEIKQRVVFKYSQMINISDYINRWIFVTITNNRLNNSKIYINGRLIDQKPISNLGNIHAS
NNIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVITTNIYLNSSLYRGTKFIIKKYASGNKDNIVRNNDRVYINVVVKNKEYRLATNASQA
GVEKILSALEIPDVGNLSQVVVMKSKNDQGITNKCKMNLQDNNGNDIGFIGFHQFNNIAKLVASNWYNRQIERSS
RTLGCSWEFIPVDDGWGERPL
SEQ ID NO: 9
(Modified BoNT/A1 (M1144 Deletion) Polypeptide Sequence)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESNHLIDLSRYASKINI
GSKVNFDPIDKNQIQLFNLESSKIEVILKNAIVYNSMYENFSTSFWIRIPKYFNSISLNNEYTIINCMENNSGWK
VSLNYGEIIWTLQDTQEIKQRVVFKYSQMINISDYINRWIFVTITNNRLNNSKIYINGRLIDQKPISNLGNIHAS
NNIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVTTNIYLNSSLYRGTKFIIKKYASGNKDNIVRNNDRVYINVVVKNKEYRLATNASQAG
VEKILSALEIPDVGNLSQVVVMKSKNDQGITNKCKMNLQDNNGNDIGFIGFHQFNNIAKLVASNWYNRQIERSSR
TLGCSWEFIPVDDGWGERPL
SEQ ID NO: 10
(BoNT/A1 (N886K, N930K, S955K, Q991K, N1026K, N1052K, Q1229K)
Nucleic Acid Sequence)
ATGCCATTCGTCAACAAGCAATTCAACTACAAAGACCCAGTCAACGGCGTCGACATCGCATACATCAAGATTCCG
AACGCCGGTCAAATGCAGCCGGTTAAGGCTTTTAAGATCCACAACAAGATTTGGGTTATCCCGGAGCGTGACACC
TTCACGAACCCGGAAGAAGGCGATCTGAACCCGCCACCGGAAGCGAAGCAAGTCCCTGTCAGCTACTACGATTCG
ACGTACCTGAGCACGGATAACGAAAAAGATAACTACCTGAAAGGTGTGACCAAGCTGTTCGAACGTATCTACAGC
ACGGATCTGGGTCGCATGCTGCTGACTAGCATTGTTCGCGGTATCCCGTTCTGGGGTGGTAGCACGATTGACACC
GAACTGAAGGTTATCGACACTAACTGCATTAACGTTATTCAACCGGATGGTAGCTATCGTAGCGAAGAGCTGAAT
CTGGTCATCATTGGCCCGAGCGCAGACATTATCCAATTCGAGTGCAAGAGCTTTGGTCACGAGGTTCTGAATCTG
ACCCGCAATGGCTATGGTAGCACCCAGTACATTCGTTTTTCGCCGGATTTTACCTTCGGCTTTGAAGAGAGCCTG
GAGGTTGATACCAATCCGTTGCTGGGTGCGGGCAAATTCGCTACCGATCCGGCTGTCACGCTGGCCCATGAACTG
ATCCACGCAGGCCACCGCCTGTACGGCATTGCCATCAACCCAAACCGTGTGTTCAAGGTTAATACGAATGCATAC
TACGAGATGAGCGGCCTGGAAGTCAGCTTCGAAGAACTGCGCACCTTCGGTGGCCATGACGCTAAATTCATTGAC
AGCTTGCAAGAGAATGAGTTCCGTCTGTACTACTATAACAAATTCAAAGACATTGCAAGCACGTTGAACAAGGCC
AAAAGCATCGTTGGTACTACCGCGTCGTTGCAGTATATGAAGAATGTGTTTAAAGAGAAGTACCTGCTGTCCGAG
GATACCTCCGGCAAGTTTAGCGTTGATAAGCTGAAGTTTGACAAACTGTACAAGATGCTGACCGAGATTTACACC
GAGGACAACTTTGTGAAATTCTTCAAAGTGTTGAATCGTAAAACCTATCTGAATTTTGACAAAGCGGTTTTCAAG
ATTAACATCGTGCCGAAGGTGAACTACACCATCTATGACGGTTTTAACCTGCGTAACACCAACCTGGCGGCGAAC
TTTAACGGTCAGAATACGGAAATCAACAACATGAATTTCACGAAGTTGAAGAACTTCACGGGTCTGTTCGAGTTC
TATAAGCTGCTGTGCGTGCGCGGTATCATCACCAGCAAAACCAAAAGCCTGGACAAAGGCTACAACAAGGCGCTG
AATGACCTGTGCATTAAGGTAAACAATTGGGATCTGTTCTTTTCGCCATCCGAAGATAATTTTACCAACGACCTG
AACAAGGGTGAAGAAATCACCAGCGATACGAATATTGAAGCAGCGGAAGAGAATATCAGCCTGGATCTGATCCAG
CAGTACTATCTGACCTTTAACTTCGACAATGAACCGGAGAACATTAGCATTGAGAATCTGAGCAGCGACATTATC
GGTCAGCTGGAACTGATGCCGAATATCGAACGTTTCCCGAACGGCAAAAAGTACGAGCTGGACAAGTACACTATG
TTCCATTACCTGCGTGCACAGGAGTTTGAACACGGTAAAAGCCGTATCGCGCTGACCAACAGCGTTAACGAGGCC
CTGCTGAACCCGAGCCGTGTCTATACCTTCTTCAGCAGCGACTATGTTAAGAAAGTGAACAAAGCCACTGAGGCC
GCGATGTTCCTGGGCTGGGTGGAACAGCTGGTATATGACTTCACGGACGAGACGAGCGAAGTGAGCACTACCGAC
AAAATTGCTGATATTACCATCATTATCCCGTATATTGGTCCGGCACTGAACATTGGCAACATGCTGTACAAAGAC
GATTTTGTGGGTGCCCTGATCTTCTCCGGTGCCGTGATTCTGCTGGAGTTCATTCCGGAGATTGCGATCCCGGTG
TTGGGTACCTTCGCGCTGGTGTCCTACATCGCGAATAAGGTTCTGACGGTTCAGACCATCGATAACGCGCTGTCG
AAACGTAATGAAAAATGGGACGAGGTTTACAAATACATTGTTACGAATTGGCTGGCGAAAGTCAATACCCAGATC
GACCTGATCCGTAAGAAAATGAAAGAGGCGCTGGAGAATCAGGCGGAGGCCACCAAAGCAATTATCAACTACCAA
TACAACCAGTACACGGAAGAAGAGAAGAATAACATTAACTTCAATATCGATGATTTGAGCAGCAAGCTGAATGAA
TCTATCAACAAAGCGATGATCAATATCAACAAGTTTTTGAATCAGTGTAGCGTTTCGTACCTGATGAATAGCATG
ATTCCGTATGGCGTCAAACGTCTGGAGGACTTCGACGCCAGCCTGAAAGATGCGTTGCTGAAATACATTTACGAC
AATCGTGGTACGCTGATTGGCCAAGTTGACCGCTTGAAAGACAAAGTTAACAATACCCTGAGCACCGACATCCCA
TTTCAACTGAGCAAGTATGTTGATAATCAACGTCTGTTGAGCACTTTCACCGAGTATATCAAAAACATCATCAAT
ACTAGCATTCTGAACCTGCGTTACGAGAGCAAGCATCTGATTGATCTGAGCCGTTATGCTAGCAAGATCAACATC
GGTAGCAAGGTCAATTTTGACCCGATCGATAAGAACCAGATCCAGCTGTTTAATCTGGAATCGAGCAAAATTGAG
GTTATCCTGAAAAAGGCCATTGTCTACAACTCCATGTACGAGAATTTCTCCACCAGCTTCTGGATTCGCATCCCG
AAATACTTCAACAAGATTAGCCTGAACAACGAGTATACTATCATCAACTGTATGGAGAACAACAGCGGTTGGAAG
GTGTCTCTGAACTATGGTGAGATCATTTGGACCTTGCAGGACACCAAAGAGATCAAGCAGCGCGTCGTGTTCAAG
TACTCTCAAATGATCAACATTTCCGATTACATTAATCGTTGGATCTTCGTGACCATTACGAATAACCGTCTGAAT
AAGAGCAAGATTTACATCAATGGTCGCTTGATCGATCAGAAACCGATTAGCAACCTGGGTAATATCCACGCAAGC
AACAAGATTATGTTCAAATTGGACGGTTGCCGCGATACCCATCGTTATATCTGGATCAAGTATTTCAACCTGTTT
GATAAAGAACTGAATGAGAAGGAGATCAAAGATTTGTATGACAACCAATCTAACAGCGGCATTTTGAAGGACTTC
TGGGGCGATTATCTGCAATACGATAAGCCGTACTATATGCTGAACCTGTATGATCCGAACAAATATGTGGATGTC
AATAATGTGGGTATTCGTGGTTACATGTATTTGAAGGGTCCGCGTGGCAGCGTTATGACGACCAACATTTACCTG
AACTCTAGCCTGTACCGTGGTACGAAATTCATCATTAAGAAATATGCCAGCGGCAACAAAGATAACATTGTGCGT
AATAACGATCGTGTCTACATCAACGTGGTCGTGAAGAATAAAGAGTACCGTCTGGCGACCAACGCTTCGCAGGCG
GGTGTTGAGAAAATTCTGAGCGCGTTGGAGATCCCTGATGTCGGTAATCTGAGCCAAGTCGTGGTTATGAAGAGC
AAGAACGACAAGGGTATCACTAACAAGTGCAAGATGAACCTGCAAGACAACAATGGTAACGACATCGGCTTTATT
GGTTTCCACCAGTTCAACAATATTGCTAAACTGGTAGCGAGCAATTGGTACAATCGTCAGATTGAGCGCAGCAGC
CGTACTTTGGGCTGTAGCTGGGAGTTTATCCCGGTCGATGATGGTTGGGGCGAACGTCCGCTG
SEQ ID NO: 11
(BoNT/A1 (N886K, N930K, S955K, Q991K, N1026K, N1052K, Q1229K)
Polypeptide Sequence)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESKHLIDLSRYASKINI
GSKVNFDPIDKNQIQLFNLESSKIEVILKKAIVYNSMYENFSTSFWIRIPKYFNKISLNNEYTIINCMENNSGWK
VSLNYGEIIWTLQDTKEIKQRVVFKYSQMINISDYINRWIFVTITNNRLNKSKIYINGRLIDQKPISNLGNIHAS
NKIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVMTTNIYLNSSLYRGTKFIIKKYASGNKDNIVRNNDRVYINVVVKNKEYRLATNASQA
GVEKILSALEIPDVGNLSQVVVMKSKNDKGITNKCKMNLQDNNGNDIGFIGFHQFNNIAKLVASNWYNRQIERSS
RTLGCSWEFIPVDDGWGERPL
SEQ ID NO: 12 (Modified BoNT/A1 (N886K, N930K, S955K, Q991K, N1026K, N1052K,
Q1229K, M1144V) Polypeptide Sequence)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESKHLIDLSRYASKINI
GSKVNFDPIDKNQIQLENLESSKIEVILKKAIVYNSMYENFSTSFWIRIPKYFNKISLNNEYTIINCMENNSGWK
VSLNYGEIIWTLQDTKEIKQRVVFKYSQMINISDYINRWIFVTITNNRLNKSKIYINGRLIDQKPISNLGNIHAS
NKIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVVTTNIYLNSSLYRGTKFIIKKYASGNKDNIVRNNDRVYINVVVKNKEYRLATNASQA
GVEKILSALEIPDVGNLSQVVVMKSKNDKGITNKCKMNLQDNNGNDIGFIGFHQFNNIAKLVASNWYNRQIERSS
RTLGCSWEFIPVDDGWGERPL
SEQ ID NO: 13
(Modified BoNT/A1 (N886K, N930K, S955K, Q991K, N1026K, N1052K,
Q1229K, M1144G) Polypeptide Sequence)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESKHLIDLSRYASKINI
GSKVNFDPIDKNQIQLFNLESSKIEVILKKAIVYNSMYENFSTSFWIRIPKYFNKISLNNEYTIINCMENNSGWK
VSLNYGEIIWTLQDTKEIKQRVVFKYSQMINISDYINRWIFVTITNNRLNKSKIYINGRLIDQKPISNLGNIHAS
NKIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVGTTNIYLNSSLYRGTKFIIKKYASGNKDNIVRNNDRVYINVVVKNKEYRLATNASQA
GVEKILSALEIPDVGNLSQVVVMKSKNDKGITNKCKMNLQDNNGNDIGFIGFHQFNNIAKLVASNWYNRQIERSS
RTLGCSWEFIPVDDGWGERPL
SEQ ID NO: 14
(Modified BoNT/A1 (N886K, N930K, S955K, Q991K, N1026K, N1052K,
Q1229K, M1144L) Polypeptide Sequence)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESKHLIDLSRYASKINI
GSKVNFDPIDKNQIQLFNLESSKIEVILKKAIVYNSMYENFSTSFWIRIPKYFNKISLNNEYTIINCMENNSGWK
VSLNYGEIIWTLQDTKEIKQRVVFKYSQMINISDYINRWIFVTITNNRLNKSKIYINGRLIDQKPISNLGNIHAS
NKIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVLTTNIYLNSSLYRGTKFIIKKYASGNKDNIVRNNDRVYINVVVKNKEYRLATNASQA
GVEKILSALEIPDVGNLSQVVVMKSKNDKGITNKCKMNLQDNNGNDIGFIGFHQFNNIAKLVASNWYNRQIERSS
RTLGCSWEFIPVDDGWGERPL
SEQ ID NO: 15
(Modified BoNT/A1 (N886K, N930K, S955K, Q991K, N1026K, N1052K,
Q1229K, M1144T) Polypeptide Sequence)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESKHLIDLSRYASKINI
GSKVNFDPIDKNQIQLFNLESSKIEVILKKAIVYNSMYENFSTSFWIRIPKYFNKISLNNEYTIINCMENNSGWK
VSLNYGEIIWTLQDTKEIKQRVVFKYSQMINISDYINRWIFVTITNNRLNKSKIYINGRLIDQKPISNLGNIHAS
NKIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVTTTNIYLNSSLYRGTKFIIKKYASGNKDNIVRNNDRVYINVVVKNKEYRLATNASQA
GVEKILSALEIPDVGNLSQVVVMKSKNDKGITNKCKMNLQDNNGNDIGFIGFHQFNNIAKLVASNWYNRQIERSS
RTLGCSWEFIPVDDGWGERPL
SEQ ID NO: 16
(Modified BoNT/A1 (N886K, N930K, S955K, Q991K, N1026K, N1052K,
Q1229K, M1144A) Polypeptide Sequence)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESKHLIDLSRYASKINI
GSKVNFDPIDKNQIQLFNLESSKIEVILKKAIVYNSMYENFSTSFWIRIPKYFNKISLNNEYTIINCMENNSGWK
VSLNYGEIIWTLQDTKEIKQRVVFKYSQMINISDYINRWIFVTITNNRLNKSKIYINGRLIDQKPISNLGNIHAS
NKIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVATTNIYLNSSLYRGTKFIIKKYASGNKDNIVRNNDRVYINVVVKNKEYRLATNASQA
GVEKILSALEIPDVGNLSQVVVMKSKNDKGITNKCKMNLQDNNGNDIGFIGFHQENNIAKLVASNWYNRQIERSS
RTLGCSWEFIPVDDGWGERPL
SEQ ID NO: 17
(Modified BoNT/A1 (N886K, N930K, S955K, Q991K, N1026K, N1052K,
Q1229K, M11441) Polypeptide Sequence)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESKHLIDLSRYASKINI
GSKVNFDPIDKNQIQLFNLESSKIEVILKKAIVYNSMYENFSTSFWIRIPKYFNKISLNNEYTIINCMENNSGWK
VSLNYGEIIWTLQDTKEIKQRVVFKYSQMINISDYINRWIFVTITNNRLNKSKIYINGRLIDQKPISNLGNIHAS
NKIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVITTNIYLNSSLYRGTKFIIKKYASGNKDNIVRNNDRVYINVVVKNKEYRLATNASQA
GVEKILSALEIPDVGNLSQVVVMKSKNDKGITNKCKMNLQDNNGNDIGFIGFHQFNNIAKLVASNWYNRQIERSS
RTLGCSWEFIPVDDGWGERPL
SEQ ID NO: 18
(Modified BoNT/A1 (N886K, N930K, S955K, Q991K, N1026K, N1052K,
Q1229K, M1144 Deletion) Polypeptide Sequence)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESKHLIDLSRYASKINI
GSKVNFDPIDKNQIQLFNLESSKIEVILKKAIVYNSMYENFSTSFWIRIPKYFNKISLNNEYTIINCMENNSGWK
VSLNYGEIIWTLQDTKEIKQRVVFKYSQMINISDYINRWIFVTITNNRLNKSKIYINGRLIDQKPISNLGNIHAS
NKIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVTTNIYLNSSLYRGTKFIIKKYASGNKDNIVRNNDRVYINVVVKNKEYRLATNASQAG
VEKILSALEIPDVGNLSQVVVMKSKNDKGITNKCKMNLQDNNGNDIGFIGFHQFNNIAKLVASNWYNRQIERSSR
TLGCSWEFIPVDDGWGERPL
SEQ ID NO: 19
(BoNT/A1 (N954K, N930K, S955K, Q991K, N1026K, N1052K, Q1229K)
Nucleic Acid Sequence)
ATGCCATTCGTCAACAAGCAATTCAACTACAAAGACCCAGTCAACGGCGTCGACATCGCATACATCAAGATTCCG
AACGCCGGTCAAATGCAGCCGGTTAAGGCTTTTAAGATCCACAACAAGATTTGGGTTATCCCGGAGCGTGACACC
TTCACGAACCCGGAAGAAGGCGATCTGAACCCGCCACCGGAAGCGAAGCAAGTCCCTGTCAGCTACTACGATTCG
ACGTACCTGAGCACGGATAACGAAAAAGATAACTACCTGAAAGGTGTGACCAAGCTGTTCGAACGTATCTACAGC
ACGGATCTGGGTCGCATGCTGCTGACTAGCATTGTTCGCGGTATCCCGTTCTGGGGTGGTAGCACGATTGACACC
GAACTGAAGGTTATCGACACTAACTGCATTAACGTTATTCAACCGGATGGTAGCTATCGTAGCGAAGAGCTGAAT
CTGGTCATCATTGGCCCGAGCGCAGACATTATCCAATTCGAGTGCAAGAGCTTTGGTCACGAGGTTCTGAATCTG
ACCCGCAATGGCTATGGTAGCACCCAGTACATTCGTTTTTCGCCGGATTTTACCTTCGGCTTTGAAGAGAGCCTG
GAGGTTGATACCAATCCGTTGCTGGGTGCGGGCAAATTCGCTACCGATCCGGCTGTCACGCTGGCCCATGAACTG
ATCCACGCAGGCCACCGCCTGTACGGCATTGCCATCAACCCAAACCGTGTGTTCAAGGTTAATACGAATGCATAC
TACGAGATGAGCGGCCTGGAAGTCAGCTTCGAAGAACTGCGCACCTTCGGTGGCCATGACGCTAAATTCATTGAC
AGCTTGCAAGAGAATGAGTTCCGTCTGTACTACTATAACAAATTCAAAGACATTGCAAGCACGTTGAACAAGGCC
AAAAGCATCGTTGGTACTACCGCGTCGTTGCAGTATATGAAGAATGTGTTTAAAGAGAAGTACCTGCTGTCCGAG
GATACCTCCGGCAAGTTTAGCGTTGATAAGCTGAAGTTTGACAAACTGTACaAGATGCTGACCGAGATTTACACC
GAGGACAACTTTGTGAAATTCTTCAAAGTGTTGAATCGTAAAACCTATCTGAATTTTGACAAAGCGGTTTTCAAG
ATTAACATCGTGCCGAAGGTGAACTACACCATCTATGACGGTTTTAACCTGCGTAACACCAACCTGGCGGCGAAC
TTTAACGGTCAGAATACGGAAATCAACAACATGAATTTCACGAAGTTGAAGAACTTCACGGGTCTGTTCGAGTTC
TATAAGCTGCTGTGCGTGCGCGGTATCATCACCAGCAAAACCAAAAGCCTGGACAAAGGCTACAACAAGGCGCTG
AATGACCTGTGCATTAAGGTAAACAATTGGGATCTGTTCTTTTCGCCATCCGAAGATAATTTTACCAACGACCTG
AACAAGGGTGAAGAAATCACCAGCGATACGAATATTGAAGCAGCGGAAGAGAATATCAGCCTGGATCTGATCCAG
CAGTACTATCTGACCTTTAACTTCGACAATGAACCGGAGAACATTAGCATTGAGAATCTGAGCAGCGACATTATC
GGTCAGCTGGAACTGATGCCGAATATCGAACGTTTCCCGAACGGCAAAAAGTACGAGCTGGACAAGTACACTATG
TTCCATTACCTGCGTGCACAGGAGTTTGAACACGGTAAAAGCCGTATCGCGCTGACCAACAGCGTTAACGAGGCC
CTGCTGAACCCGAGCCGTGTCTATACCTTCTTCAGCAGCGACTATGTTAAGAAAGTGAACAAAGCCACTGAGGCC
GCGATGTTCCTGGGCTGGGTGGAACAGCTGGTATATGACTTCACGGACGAGACGAGCGAAGTGAGCACTACCGAC
AAAATTGCTGATATTACCATCATTATCCCGTATATTGGTCCGGCACTGAACATTGGCAACATGCTGTACAAAGAC
GATTTTGTGGGTGCCCTGATCTTCTCCGGTGCCGTGATTCTGCTGGAGTTCATTCCGGAGATTGCGATCCCGGTG
TTGGGTACCTTCGCGCTGGTGTCCTACATCGCGAATAAGGTTCTGACGGTTCAGACCATCGATAACGCGCTGTCG
AAACGTAATGAAAAATGGGACGAGGTTTACAAATACATTGTTACGAATTGGCTGGCGAAAGTCAATACCCAGATC
GACCTGATCCGTAAGAAAATGAAAGAGGCGCTGGAGAATCAGGCGGAGGCCACCAAAGCAATTATCAACTACCAA
TACAACCAGTACACGGAAGAAGAGAAGAATAACATTAACTTCAATATCGATGATTTGAGCAGCAAGCTGAATGAA
TCTATCAACAAAGCGATGATCAATATCAACAAGTTTTTGAATCAGTGTAGCGTTTCGTACCTGATGAATAGCATG
ATTCCGTATGGCGTCAAACGTCTGGAGGACTTCGACGCCAGCCTGAAAGATGCGTTGCTGAAATACATTTACGAC
AaTCGTGGTACGCTGATTGGCCAAGTTGACCGCTTGAAAGACAAAGTTAACAATACCCTGAGCACCGACATCCCA
TTTCAACTGAGCAAGTATGTTGATAATCAACGTCTGTTGAGCACTTTCACCGAGTATATCAAAAACATCATCAAT
ACTAGCATTCTGAACCTGCGTTACGAGAGCAATCATCTGATTGATCTGAGCCGTTATGCTAGCAAGATCAACATC
GGTAGCAAGGTCAATTTTGACCCGATCGATAAGAACCAGATCCAGCTGTTTAATCTGGAATCGAGCAAAATTGAG
GTTATCCTGAAAAAGGCCATTGTCTACAACTCCATGTACGAGAATTTCTCCACCAGCTTCTGGATTCGCATCCCG
AAATACTTCAAGAAGATTAGCCTGAACAACGAGTATACTATCATCAACTGTATGGAGAACAACAGCGGTTGGAAG
GTGTCTCTGAACTATGGTGAGATCATTTGGACCTTGCAGGACACCAAAGAGATCAAGCAGCGCGTCGTGTTCAAG
TACTCTCAAATGATCAACATTTCCGATTACATTAATCGTTGGATCTTCGTGACCATTACGAATAACCGTCTGAAT
AAGAGCAAGATTTACATCAATGGTCGCTTGATCGATCAGAAACCGATTAGCAACCTGGGTAATATCCACGCAAGC
AACAAGATTATGTTCAAATTGGACGGTTGCCGCGATACCCATCGTTATATCTGGATCAAGTATTTCAACCTGTTT
GATAAAGAACTGAATGAGAAGGAGATCAAAGATTTGTATGACAACCAATCTAACAGCGGCATTTTGAAGGACTTC
TGGGGCGATTATCTGCAATACGATAAGCCGTACTATATGCTGAACCTGTATGATCCGAACAAATATGTGGATGTC
AATAATGTGGGTATTCGTGGTTACATGTATTTGAAGGGTCCGCGTGGCAGCGTTATGACGACCAACATTTACCTG
AACTCTAGCCTGTACCGTGGTACGAAATTCATCATTAAGAAATATGCCAGCGGCAACAAAGATAACATTGTGCGT
AATAACGATCGTGTCTACATCAACGTGGTCGTGAAGAATAAAGAGTACCGTCTGGCGACCAACGCTTCGCAGGCG
GGTGTTGAGAAAATTCTGAGCGCGTTGGAGATCCCTGATGTCGGTAATCTGAGCCAAGTCGTGGTTATGAAGAGC
AAGAACGACAAGGGTATCACTAACAAGTGCAAGATGAACCTGCAAGACAACAATGGTAACGACATCGGCTTTATT
GGTTTCCACCAGTTCAACAATATTGCTAAACTGGTAGCGAGCAATTGGTACAATCGTCAGATTGAGCGCAGCAGC
CGTACTTTGGGCTGTAGCTGGGAGTTTATCCCGGTCGATGATGGTTGGGGCGAACGTCCGCTG
SEQ ID NO: 20
(BoNT/A1 (N954K, N930K, S955K, Q991K, N1026K, N1052K, Q1229K)
Polypeptide Sequence)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESNHLIDLSRYASKINI
GSKVNFDPIDKNQIQLFNLESSKIEVILKKAIVYNSMYENFSTSFWIRIPKYFKKISLNNEYTIINCMENNSGWK
VSLNYGEIIWTLQDTKEIKQRVVFKYSQMINISDYINRWIFVTITNNRLNKSKIYINGRLIDQKPISNLGNIHAS
NKIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVMTTNIYLNSSLYRGTKFIIKKYASGNKDNIVRNNDRVYINVVVKNKEYRLATNASQA
GVEKILSALEIPDVGNLSQVVVMKSKNDKGITNKCKMNLQDNNGNDIGFIGFHQFNNIAKLVASNWYNRQIERSS
RTLGCSWEFIPVDDGWGERPL
SEQ ID NO: 21
(Modified BoNT/A1 (N954K, N930K, S955K, Q991K, N1026K, N1052K,
Q1229K, M1144V) Polypeptide Sequence)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESNHLIDLSRYASKINI
GSKVNFDPIDKNQIQLFNLESSKIEVILKKAIVYNSMYENFSTSFWIRIPKYFKKISLNNEYTIINCMENNSGWK
VSLNYGEIIWTLQDTKEIKQRVVFKYSQMINISDYINRWIFVTITNNRLNKSKIYINGRLIDQKPISNLGNIHAS
NKIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVVTTNIYLNSSLYRGTKFIIKKYASGNKDNIVRNNDRVYINVVVKNKEYRLATNASQA
GVEKILSALEIPDVGNLSQVVVMKSKNDKGITNKCKMNLQDNNGNDIGFIGFHQFNNIAKLVASNWYNRQIERSS
RTLGCSWEFIPVDDGWGERPL
SEQ ID NO: 22
(Modified BoNT/A1 (N954K, N930K, S955K, Q991K, N1026K, N1052K,
Q1229K, M1144G) Polypeptide Sequence)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESNHLIDLSRYASKINI
GSKVNFDPIDKNQIQLFNLESSKIEVILKKAIVYNSMYENFSTSFWIRIPKYFKKISLNNEYTIINCMENNSGWK
VSLNYGEIIWTLQDTKEIKQRVVFKYSQMINISDYINRWIFVTITNNRLNKSKIYINGRLIDQKPISNLGNIHAS
NKIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVGTTNIYLNSSLYRGTKFIIKKYASGNKDNIVRNNDRVYINVVVKNKEYRLATNASQA
GVEKILSALEIPDVGNLSQVVVMKSKNDKGITNKCKMNLQDNNGNDIGFIGFHQFNNIAKLVASNWYNRQIERSS
RTLGCSWEFIPVDDGWGERPL
SEQ ID NO: 23
(Modified BoNT/A1 (N954K, N930K, S955K, Q991K, N1026K, N1052K,
Q1229K, M1144L) Polypeptide Sequence)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESNHLIDLSRYASKINI
GSKVNFDPIDKNQIQLFNLESSKIEVILKKAIVYNSMYENFSTSFWIRIPKYFKKISLNNEYTIINCMENNSGWK
VSLNYGEIIWTLQDTKEIKQRVVFKYSQMINISDYINRWIFVTITNNRLNKSKIYINGRLIDQKPISNLGNIHAS
NKIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVLTTNIYLNSSLYRGTKFIIKKYASGNKDNIVRNNDRVYINVVVKNKEYRLATNASQA
GVEKILSALEIPDVGNLSQVVVMKSKNDKGITNKCKMNLQDNNGNDIGFIGFHQFNNIAKLVASNWYNRQIERSS
RTLGCSWEFIPVDDGWGERPL
SEQ ID NO: 24
(Modified BoNT/A1 (N954K, N930K, S955K, Q991K, N1026K, N1052K,
Q1229K, M1144T) Polypeptide Sequence)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESNHLIDLSRYASKINI
GSKVNFDPIDKNQIQLFNLESSKIEVILKKAIVYNSMYENFSTSFWIRIPKYFKKISLNNEYTIINCMENNSGWK
VSLNYGEIIWTLQDTKEIKQRVVFKYSQMINISDYINRWIFVTITNNRLNKSKIYINGRLIDQKPISNLGNIHAS
NKIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVTTTNIYLNSSLYRGTKFIIKKYASGNKDNIVRNNDRVYINVVVKNKEYRLATNASQA
GVEKILSALEIPDVGNLSQVVVMKSKNDKGITNKCKMNLQDNNGNDIGFIGFHQFNNIAKLVASNWYNRQIERSS
RTLGCSWEFIPVDDGWGERPL
SEQ ID NO: 25
(Modified BoNT/A1 (N954K, N930K, S955K, Q991K, N1026K, N1052K,
Q1229K, M1144A) Polypeptide Sequence)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESNHLIDLSRYASKINI
GSKVNFDPIDKNQIQLFNLESSKIEVILKKAIVYNSMYENFSTSFWIRIPKYFKKISLNNEYTIINCMENNSGWK
VSLNYGEIIWTLQDTKEIKQRVVFKYSQMINISDYINRWIFVTITNNRLNKSKIYINGRLIDQKPISNLGNIHAS
NKIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVATTNIYLNSSLYRGTKFIIKKYASGNKDNIVRNNDRVYINVVVKNKEYRLATNASQA
GVEKILSALEIPDVGNLSQVVVMKSKNDKGITNKCKMNLQDNNGNDIGFIGFHQFNNIAKLVASNWYNRQIERSS
RTLGCSWEFIPVDDGWGERPL
SEQ ID NO: 26
(Modified BoNT/A1 (N954K, N930K, S955K, Q991K, N1026K, N1052K,
Q1229K, M11441) Polypeptide Sequence)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTINKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESNHLIDLSRYASKINI
GSKVNFDPIDKNQIQLFNLESSKIEVILKKAIVYNSMYENFSTSFWIRIPKYFKKISLNNEYTIINCMENNSGWK
VSLNYGEIIWTLQDTKEIKQRVVFKYSQMINISDYINRWIFVTITNNRLNKSKIYINGRLIDQKPISNLGNIHAS
NKIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVITTNIYLNSSLYRGTKFIIKKYASGNKDNIVRNNDRVYINVVVKNKEYRLATNASQA
GVEKILSALEIPDVGNLSQVVVMKSKNDKGITNKCKMNLQDNNGNDIGFIGFHQFNNIAKLVASNWYNRQIERSS
RTLGCSWEFIPVDDGWGERPL
SEQ ID NO: 27
(Modified BoNT/A1 (N954K, N930K, S955K, Q991K, N1026K, N1052K,
Q1229K, M1144 Deletion) Polypeptide Sequence)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESNHLIDLSRYASKINI
GSKVNFDPIDKNQIQLFNLESSKIEVILKKAIVYNSMYENFSTSFWIRIPKYFKKISLNNEYTIINCMENNSGWK
VSLNYGEIIWTLQDTKEIKQRVVFKYSQMINISDYINRWIFVTITNNRLNKSKIYINGRLIDQKPISNLGNIHAS
NKIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVTTNIYLNSSLYRGTKFIIKKYASGNKDNIVRNNDRVYINVVVKNKEYRLATNASQAG
VEKILSALEIPDVGNLSQVVVMKSKNDKGITNKCKMNLQDNNGNDIGFIGFHQFNNIAKLVASNWYNRQIERSSR
TLGCSWEFIPVDDGWGERPL
SEQ ID NO: 28
(BoNT/A1 (N930K, S955K, Q991K, N1026K, N1052K, Q1229K, N1025K)
Nucleic Acid Sequence)
ATGCCATTCGTCAACAAGCAATTCAACTACAAAGACCCAGTCAACGGCGTCGACATCGCATACATCAAGATTCCG
AACGCCGGTCAAATGCAGCCGGTTAAGGCTTTTAAGATCCACAACAAGATTTGGGTTATCCCGGAGCGTGACACC
TTCACGAACCCGGAAGAAGGCGATCTGAACCCGCCACCGGAAGCGAAGCAAGTCCCTGTCAGCTACTACGATTCG
ACGTACCTGAGCACGGATAACGAAAAAGATAACTACCTGAAAGGTGTGACCAAGCTGTTCGAACGTATCTACAGC
ACGGATCTGGGTCGCATGCTGCTGACTAGCATTGTTCGCGGTATCCCGTTCTGGGGTGGTAGCACGATTGACACC
GAACTGAAGGTTATCGACACTAACTGCATTAACGTTATTCAACCGGATGGTAGCTATCGTAGCGAAGAGCTGAAT
CTGGTCATCATTGGCCCGAGCGCAGACATTATCCAATTCGAGTGCAAGAGCTTTGGTCACGAGGTTCTGAATCTG
ACCCGCAATGGCTATGGTAGCACCCAGTACATTCGTTTTTCGCCGGATTTTACCTTCGGCTTTGAAGAGAGCCTG
GAGGTTGATACCAATCCGTTGCTGGGTGCGGGCAAATTCGCTACCGATCCGGCTGTCACGCTGGCCCATGAACTG
ATCCACGCAGGCCACCGCCTGTACGGCATTGCCATCAACCCAAACCGTGTGTTCAAGGTTAATACGAATGCATAC
TACGAGATGAGCGGCCTGGAAGTCAGCTTCGAAGAACTGCGCACCTTCGGTGGCCATGACGCTAAATTCATTGAC
AGCTTGCAAGAGAATGAGTTCCGTCTGTACTACTATAACAAATTCAAAGACATTGCAAGCACGTTGAACAAGGCC
AAAAGCATCGTTGGTACTACCGCGTCGTTGCAGTATATGAAGAATGTGTTTAAAGAGAAGTACCTGCTGTCCGAG
GATACCTCCGGCAAGTTTAGCGTTGATAAGCTGAAGTTTGACAAACTGTACAAGATGCTGACCGAGATTTACACC
GAGGACAACTTTGTGAAATTCTTCAAAGTGTTGAATCGTAAAACCTATCTGAATTTTGACAAAGCGGTTTTCAAG
ATTAACATCGTGCCGAAGGTGAACTACACCATCTATGACGGTTTTAACCTGCGTAACACCAACCTGGCGGCGAAC
TTTAACGGTCAGAATACGGAAATCAACAACATGAATTTCACGAAGTTGAAGAACTTCACGGGTCTGTTCGAGTTC
TATAAGCTGCTGTGCGTGCGCGGTATCATCACCAGCAAAACCAAAAGCCTGGACAAAGGCTACAACAAGGCGCTG
AATGACCTGTGCATTAAGGTAAACAATTGGGATCTGTTCTTTTCGCCATCCGAAGATAATTTTACCAACGACCTG
AACAAGGGTGAAGAAATCACCAGCGATACGAATATTGAAGCAGCGGAAGAGAATATCAGCCTGGATCTGATCCAG
CAGTACTATCTGACCTTTAACTTCGACAATGAACCGGAGAACATTAGCATTGAGAATCTGAGCAGCGACATTATC
GGTCAGCTGGAACTGATGCCGAATATCGAACGTTTCCCGAACGGCAAAAAGTACGAGCTGGACAAGTACACTATG
TTCCATTACCTGCGTGCACAGGAGTTTGAACACGGTAAAAGCCGTATCGCGCTGACCAACAGCGTTAACGAGGCC
CTGCTGAACCCGAGCCGTGTCTATACCTTCTTCAGCAGCGACTATGTTAAGAAAGTGAACAAAGCCACTGAGGCC
GCGATGTTCCTGGGCTGGGTGGAACAGCTGGTATATGACTTCACGGACGAGACGAGCGAAGTGAGCACTACCGAC
AAAATTGCTGATATTACCATCATTATCCCGTATATTGGTCCGGCACTGAACATTGGCAACATGCTGTACAAAGAC
GATTTTGTGGGTGCCCTGATCTTCTCCGGTGCCGTGATTCTGCTGGAGTTCATTCCGGAGATTGCGATCCCGGTG
TTGGGTACCTTCGCGCTGGTGTCCTACATCGCGAATAAGGTTCTGACGGTTCAGACCATCGATAACGCGCTGTCG
AAACGTAATGAAAAATGGGACGAGGTTTACAAATACATTGTTACGAATTGGCTGGCGAAAGTCAATACCCAGATC
GACCTGATCCGTAAGAAAATGAAAGAGGCGCTGGAGAATCAGGCGGAGGCCACCAAAGCAATTATCAACTACCAA
TACAACCAGTACACGGAAGAAGAGAAGAATAACATTAACTTCAATATCGATGATTTGAGCAGCAAGCTGAATGAA
TCTATCAACAAAGCGATGATCAATATCAACAAGTTTTTGAATCAGTGTAGCGTTTCGTACCTGATGAATAGCATG
ATTCCGTATGGCGTCAAACGTCTGGAGGACTTCGACGCCAGCCTGAAAGATGCGTTGCTGAAATACATTTACGAC
AATCGTGGTACGCTGATTGGCCAAGTTGACCGCTTGAAAGACAAAGTTAACAATACCCTGAGCACCGACATCCCA
TTTCAACTGAGCAAGTATGTTGATAATCAACGTCTGTTGAGCACTTTCACCGAGTATATCAAAAACATCATCAAT
ACTAGCATTCTGAACCTGCGTTACGAGAGCAATCATCTGATTGATCTGAGCCGTTATGCTAGCAAGATCAACATC
GGTAGCAAGGTCAATTTTGACCCGATCGATAAGAACCAGATCCAGCTGTTTAATCTGGAATCGAGCAAAATTGAG
GTTATCCTGAAAAAGGCCATTGTCTACAACTCCATGTACGAGAATTTCTCCACCAGCTTCTGGATTCGCATCCCG
AAATACTTCAACAAGATTAGCCTGAACAACGAGTATACTATCATCAACTGTATGGAGAACAACAGCGGTTGGAAG
GTGTCTCTGAACTATGGTGAGATCATTTGGACCTTGCAGGACACCAAAGAGATCAAGCAGCGCGTCGTGTTCAAG
TACTCTCAAATGATCAACATTTCCGATTACATTAATCGTTGGATCTTCGTGACCATTACGAATAACCGTCTGAAG
AAGAGCAAGATTTACATCAATGGTCGCTTGATCGATCAGAAACCGATTAGCAACCTGGGTAATATCCACGCAAGC
AACAAGATTATGTTCAAATTGGACGGTTGCCGCGATACCCATCGTTATATCTGGATCAAGTATTTCAACCTGTTT
GATAAAGAACTGAATGAGAAGGAGATCAAAGATTTGTATGACAACCAATCTAACAGCGGCATTTTGAAGGACTTC
TGGGGCGATTATCTGCAATACGATAAGCCGTACTATATGCTGAACCTGTATGATCCGAACAAATATGTGGATGTC
AATAATGTGGGTATTCGTGGTTACATGTATTTGAAGGGTCCGCGTGGCAGCGTTATGACGACCAACATTTACCTG
AACTCTAGCCTGTACCGTGGTACGAAATTCATCATTAAGAAATATGCCAGCGGCAACAAAGATAACATTGTGCGT
AATAACGATCGTGTCTACATCAACGTGGTCGTGAAGAATAAAGAGTACCGTCTGGCGACCAACGCTTCGCAGGCG
GGTGTTGAGAAAATTCTGAGCGCGTTGGAGATCCCTGATGTCGGTAATCTGAGCCAAGTCGTGGTTATGAAGAGC
AAGAACGACAAGGGTATCACTAACAAGTGCAAGATGAACCTGCAAGACAACAATGGTAACGACATCGGCTTTATT
GGTTTCCACCAGTTCAACAATATTGCTAAACTGGTAGCGAGCAATTGGTACAATCGTCAGATTGAGCGCAGCAGC
CGTACTTTGGGCTGTAGCTGGGAGTTTATCCCGGTCGATGATGGTTGGGGCGAACGTCCGCTG
SEQ ID NO: 29
(BoNT/A1 (N930K, S955K, Q991K, N1026K, N1052K, Q1229K, N1025K)
Polypeptide Sequence)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTINKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESNHLIDLSRYASKINI
GSKVNFDPIDKNQIQLFNLESSKIEVILKKAIVYNSMYENFSTSFWIRIPKYFNKISLNNEYTIINCMENNSGWK
VSLNYGEIIWTLQDTKEIKQRVVFKYSQMINISDYINRWIFVTITNNRLKKSKIYINGRLIDQKPISNLGNIHAS
NKIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVMTTNIYLNSSLYRGTKFIIKKYASGNKDNIVRNNDRVYINVVVKNKEYRLATNASQA
GVEKILSALEIPDVGNLSQVVVMKSKNDKGITNKCKMNLQDNNGNDIGFIGFHQFNNIAKLVASNWYNRQIERSS
RTLGCSWEFIPVDDGWGERPL
SEQ ID NO: 30
(Modified BoNT/A1 (N930K, S955K, Q991K, N1026K, N1052K, Q1229K,
N1025K, M1144V) Polypeptide Sequence)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESNHLIDLSRYASKINI
GSKVNFDPIDKNQIQLFNLESSKIEVILKKAIVYNSMYENFSTSFWIRIPKYFNKISLNNEYTIINCMENNSGWK
VSLNYGEIIWTLQDTKEIKQRVVFKYSQMINISDYINRWIFVTITNNRLKKSKIYINGRLIDQKPISNLGNIHAS
NKIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVVTTNIYLNSSLYRGTKFIIKKYASGNKDNIVRNNDRVYINVVVKNKEYRLATNASQA
GVEKILSALEIPDVGNLSQVVVMKSKNDKGITNKCKMNLQDNNGNDIGFIGFHQFNNIAKLVASNWYNRQIERSS
RTLGCSWEFIPVDDGWGERPL
SEQ ID NO: 31
(Modified BoNT/A1 (N930K, S955K, Q991K, N1026K, N1052K, Q1229K,
N1025K, M1144G) Polypeptide Sequence)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESNHLIDLSRYASKINI
GSKVNFDPIDKNQIQLFNLESSKIEVILKKAIVYNSMYENFSTSFWIRIPKYFNKISLNNEYTIINCMENNSGWK
VSLNYGEIIWTLQDTKEIKQRVVFKYSQMINISDYINRWIFVTITNNRLKKSKIYINGRLIDQKPISNLGNIHAS
NKIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVGTTNIYLNSSLYRGTKFIIKKYASGNKDNIVRNNDRVYINVVVKNKEYRLATNASQA
GVEKILSALEIPDVGNLSQVVVMKSKNDKGITNKCKMNLQDNNGNDIGFIGFHQFNNIAKLVASNWYNRQIERSS
RTLGCSWEFIPVDDGWGERPL
SEQ ID NO: 32
(Modified BoNT/A1 (N930K, S955K, Q991K, N1026K, N1052K, Q1229K,
N1025K, M1144L) Polypeptide Sequence)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESNHLIDLSRYASKINI
GSKVNFDPIDKNQIQLFNLESSKIEVILKKAIVYNSMYENFSTSFWIRIPKYFNKISLNNEYTIINCMENNSGWK
VSLNYGEIIWTLQDTKEIKQRVVFKYSQMINISDYINRWIFVTITNNRLKKSKIYINGRLIDQKPISNLGNIHAS
NKIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVLTTNIYLNSSLYRGTKFIIKKYASGNKDNIVRNNDRVYINVVVKNKEYRLATNASQA
GVEKILSALEIPDVGNLSQVVVMKSKNDKGITNKCKMNLQDNNGNDIGFIGFHQFNNIAKLVASNWYNRQIERSS
RTLGCSWEFIPVDDGWGERPL
SEQ ID NO: 33
(Modified BoNT/A1 (N930K, S955K, Q991K, N1026K, N1052K, Q1229K,
N1025K, M1144T) Polypeptide Sequence)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTINKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESNHLIDLSRYASKINI
GSKVNFDPIDKNQIQLFNLESSKIEVILKKAIVYNSMYENFSTSFWIRIPKYFNKISLNNEYTIINCMENNSGWK
VSLNYGEIIWTLQDTKEIKQRVVFKYSQMINISDYINRWIFVTITNNRLKKSKIYINGRLIDQKPISNLGNIHAS
NKIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVTTTNIYLNSSLYRGTKFIIKKYASGNKDNIVRNNDRVYINVVVKNKEYRLATNASQA
GVEKILSALEIPDVGNLSQVVVMKSKNDKGITNKCKMNLQDNNGNDIGFIGFHQFNNIAKLVASNWYNRQIERSS
RTLGCSWEFIPVDDGWGERPL
SEQ ID NO: 34
(Modified BoNT/A1 (N930K, S955K, Q991K, N1026K, N1052K, Q1229K,
N1025K, M1144A) Polypeptide Sequence)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESNHLIDLSRYASKINI
GSKVNFDPIDKNQIQLFNLESSKIEVILKKAIVYNSMYENFSTSFWIRIPKYFNKISLNNEYTIINCMENNSGWK
VSLNYGEIIWTLQDTKEIKQRVVFKYSQMINISDYINRWIFVTITNNRLKKSKIYINGRLIDQKPISNLGNIHAS
NKIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVATTNIYLNSSLYRGTKFIIKKYASGNKDNIVRNNDRVYINVVVKNKEYRLATNASQA
GVEKILSALEIPDVGNLSQVVVMKSKNDKGITNKCKMNLQDNNGNDIGFIGFHQFNNIAKLVASNWYNRQIERSS
RTLGCSWEFIPVDDGWGERPL
SEQ ID NO: 35
(Modified BoNT/A1 (N930K, S955K, Q991K, N1026K, N1052K, Q1229K,
N1025K, M11441) Polypeptide Sequence)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESNHLIDLSRYASKINI
GSKVNFDPIDKNQIQLFNLESSKIEVILKKAIVYNSMYENFSTSFWIRIPKYFNKISLNNEYTIINCMENNSGWK
VSLNYGEIIWTLQDTKEIKQRVVFKYSQMINISDYINRWIFVTITNNRLKKSKIYINGRLIDQKPISNLGNIHAS
NKIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVITTNIYLNSSLYRGTKFIIKKYASGNKDNIVRNNDRVYINVVVKNKEYRLATNASQA
GVEKILSALEIPDVGNLSQVVVMKSKNDKGITNKCKMNLQDNNGNDIGFIGFHQFNNIAKLVASNWYNRQIERSS
RTLGCSWEFIPVDDGWGERPL
SEQ ID NO: 36
(Modified BoNT/A1 (N930K, S955K, Q991K, N1026K, N1052K, Q1229K,
N1025K, M1144 Deletion) Polypeptide Sequence)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESNHLIDLSRYASKINI
GSKVNFDPIDKNQIQLFNLESSKIEVILKKAIVYNSMYENFSTSFWIRIPKYFNKISLNNEYTIINCMENNSGWK
VSLNYGEIIWTLQDTKEIKQRVVFKYSQMINISDYINRWIFVTITNNRLKKSKIYINGRLIDQKPISNLGNIHAS
NKIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVTTNIYLNSSLYRGTKFIIKKYASGNKDNIVRNNDRVYINVVVKNKEYRLATNASQAG
VEKILSALEIPDVGNLSQVVVMKSKNDKGITNKCKMNLQDNNGNDIGFIGFHQFNNIAKLVASNWYNRQIERSSR
TLGCSWEFIPVDDGWGERPL
SEQ ID NO: 37
(BoNT/A1 (N1188R, D1213R, G1215R, N1216R, N1242R, N1243R, S1274R,
T1277R) Nucleic Acid Sequence)
ATGCCATTCGTCAACAAGCAATTCAACTACAAAGACCCAGTCAACGGCGTCGACATCGCATACATCAAGATTCCG
AACGCCGGTCAAATGCAGCCGGTTAAGGCTTTTAAGATCCACAACAAGATTTGGGTTATCCCGGAGCGTGACACC
TTCACGAACCCGGAAGAAGGCGATCTGAACCCGCCACCGGAAGCGAAGCAAGTCCCTGTCAGCTACTACGATTCG
ACGTACCTGAGCACGGATAACGAAAAAGATAACTACCTGAAAGGTGTGACCAAGCTGTTCGAACGTATCTACAGC
ACGGATCTGGGTCGCATGCTGCTGACTAGCATTGTTCGCGGTATCCCGTTCTGGGGTGGTAGCACGATTGACACC
GAACTGAAGGTTATCGACACTAACTGCATTAACGTTATTCAACCGGATGGTAGCTATCGTAGCGAAGAGCTGAAT
CTGGTCATCATTGGCCCGAGCGCAGACATTATCCAATTCGAGTGCAAGAGCTTTGGTCACGAGGTTCTGAATCTG
ACCCGCAATGGCTATGGTAGCACCCAGTACATTCGTTTTTCGCCGGATTTTACCTTCGGCTTTGAAGAGAGCCTG
GAGGTTGATACCAATCCGTTGCTGGGTGCGGGCAAATTCGCTACCGATCCGGCTGTCACGCTGGCCCATGAACTG
ATCCACGCAGGCCACCGCCTGTACGGCATTGCCATCAACCCAAACCGTGTGTTCAAGGTTAATACGAATGCATAC
TACGAGATGAGCGGCCTgGAAGTCAGCTTCGAAGAACTGCGCACCTTCGGTGGCCATGACGCTAAATTCATTGAC
AGCTTGCAAGAGAATGAGTTCCGTCTGTACTACTATAACAAATTCAAAGACATTGCAAGCACGTTGAACAAGGCC
AAAAGCATCGTTGGTACTACCGCGTCGTTGCAGTATATGAAGAATGTGTTTAAAGAGAAGTACCTGCTGTCCGAG
GATACCTCCGGCAAGTTTAGCGTTGATAAGCTGAAGTTTGACAAACTGTACAAGATGCTGACCGAGATTTACACC
GAGGACAACTTTGTGAAATTCTTCAAaGTGTTGAATCGTAAAACCTATCTGAATTTTGACAAAGCGGTTTTCaAG
ATTAACATCGTGCCGAAGGTGAACTACACCATCTATGACGGTTTTAACCTGCGTAACACCAACCTGGCGGCGAAC
TTTAACGGTCAGAATACGGAAATCAACAACATGAATTTCACGAAGTTGAAGAACTTCACGGGTCTGTTCGAGTTC
TATAAGCTGCTGTGCGTGCGCGGTATCATCACCAGCAAAACCAAAAGCCTGGACAAAGGCTACAACAAGGCGCTG
AATGACCTGTGCATTAAGGTAAACAATTGGGATCTGTTCTTTTCGCCATCCGAAGATAATTTTACCAACGACCTG
AACAAGGGTGAAGAAATCACCAGCGATACGAATATTGAAGCAGCGGAAGAGAATATCAGCCTGGATCTGATCCAG
CAGTACTATCTGACCTTTAACTTCGACAATGAACCGGAGAACATTAGCATTGAGAATCTGAGCAGCGACATTATC
GGTCAGCTGGAACTGATGCCGAATATCGAACGTTTCCCGAACGGCAAAAAGTACGAGCTGGACAAGTACACTATG
TTCCATTACCTGCGTGCACAGGAGTTTGAACACGGTAAAAGCCGTATCGCGCTGACCAACAGCGTTAACGAGGCC
CTGCTGAACCCGAGCCGTGTCTATACCTTCTTCAGCAGCGACTATGTTAAGAAAGTGAACAAAGCCACTGAGGCC
GCGATGTTCCTGGGCTGGGTGGAACAGCTGGTATATGACTTCACGGACGAGACGAGCGAAGTGAGCACTACCGAC
AAAaTTGCTGATaTTACCATCATTATCCCGTATATTGGTCCGGCACTGAACATTGGCAACATGCTGTACAAAGAC
GATTTTGTGGGTGCCCTGATCTTCTCCGGTGCCGTGATTCTGCTGGAGTTCATTCCGGAGATTGCGATCCCGGTG
TTGGGTACCTTCGCGCTGGTGTCCTACATCGCGAATAAGGTTCTGACGGTTCAGACCATCGATAACGCGCTGTCG
AAACGTAATGAAAAATGGGACGAGGTTTACAAATACATTGTTACGAATTGGCTGGCGAAAGTCaATACCCAGATC
GACCTGATCCGTAAGAAAATGAAAGAGGCGCTGGAGAATCAGGCGGAGGCCACCAAAGCAATTATCAACTACCAA
TACAACCAGTACACGGAAGAAGAGAAGAATAACATTAACTTCAATATCGATGATTTGAGCAGCAAGCTGAATGAA
TCTATCAACAAAGCGATGATCAATATCAACAAGTTTTTGAATCAGTGTAGCGTTTCGTACCTGATGAATAGCATG
ATTCCGTATGGCGTCAAACGTCTGGAGGACTTCGACGCCAGCCTGAAAGATGCGTTGCTGAAATACATTTACGAC
AATCGTGGTACGCTGATTGGCCAAGTTGACCGCTTGAAAGACAAAGTTAACAATACCCTGAGCACCGACATCCCA
TTTCAACTGAGCAAGTATGTTGATAATCAACGTCTGTTGAGCACTTTCACCGAGTATATCAAAAACATCATCAAT
ACTAGCATTCTGAACCTGCGTTACGAGAGCAATCATCTGATtGATCTGAGCCGTTATGCAAGCAAGATCAACATC
GGTAGCAAGGTCAATTTTGACCCGATCGATAAGAACCAGATCCAGCTGTTTAATCTGGAATCGAGCAAAATTGAG
GTTATCCTGAAAAACGCCATTGTCTACAACTCCATGTACGAGAATTTCTCCACCAGCTTCTGGATTCGCATCCCG
AAATACTTCAACAGCATTAGCCTGAACAACGAGTATACTATCATCAACTGTATGGAGAACAACAGCGGTTGGAAG
GTGTCTCTGAACTATGGTGAGATCATTTGGACCTTGCAGGACACCCAAGAGATCAAGCAGCGCGTCGTGTTCAAG
TACTCTCAAATGATCAACATTTCCGATTACATTAATCGTTGGATCTTCGTGACCATTACGAATAACCGTCTGAAT
AACAGCAAGATTTACATCAATGGTCGCTTGATCGATCAGAAACCGATTAGCAACCTGGGTAATATCCACGCAAGC
AACAACATTATGTTCAAATTGGACGGTTGCCGCGATACCCATCGTTATATCTGGATCAAGTATTTCAACCTGTTT
GATAAAGAACTGAATGAGAAGGAGATCAAAGATTTGTATGACAACCAATCTAACAGCGGCATTTTGAAGGACTTC
TGGGGCGATTATCTGCAATACGATAAGCCGTACTATATGCTGAACCTGTATGATCCGAACAAATATGTGGATGTC
AATAATGTGGGTATTCGTGGTTACATGTATTTGAAGGGTCCGCGTGGCAGCGTTATGACGACCAACATTTACCTG
AACTCTAGCCTGTACCGTGGTACGAAATTCATCATTAAGAAATATGCCAGCGGCAACAAAGATAACATTGTGCGT
AATAACGATCGTGTCTACATCAACGTGGTCGTGAAGCGTAAAGAGTACCGTCTGGCGACCAACGCTTCGCAGGCG
GGTGTTGAGAAAATTCTGAGCGCGTTGGAGATCCCTCGTGTCCGTCGTCTGAGCCAAGTCGTGGTTATGAAGAGC
AAGAACGACCAGGGTATCACTAACAAGTGCAAGATGAACCTGCAAGACCGTCGTGGTAACGACATCGGCTTTATT
GGTTTCCACCAGTTCAACAATATTGCTAAACTGGTAGCGAGCAATTGGTACAATCGTCAGATTGAGCGCCGTAGC
CGTCGTTTGGGCTGTAGCTGGGAGTTTATCCCGGTCGATGATGGTTGGGGCGAACGTCCGCTG
SEQ ID NO: 38
(BoNT/A1 (N1188R, D1213R, G1215R, N1216R, N1242R, N1243R, S1274R,
T1277R) Polypeptide Sequence)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTINKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESNHLIDLSRYASKINI
GSKVNFDPIDKNQIQLENLESSKIEVILKNAIVYNSMYENFSTSFWIRIPKYFNSISLNNEYTIINCMENNSGWK
VSLNYGEIIWTLQDTQEIKQRVVFKYSQMINISDYINRWIFVTITNNRLNNSKIYINGRLIDQKPISNLGNIHAS
NNIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVMTTNIYLNSSLYRGTKFIIKKYASGNKDNIVRNNDRVYINVVVKRKEYRLATNASQA
GVEKILSALEIPRVRRLSQVVVMKSKNDQGITNKCKMNLQDRRGNDIGFIGFHQFNNIAKLVASNWYNRQIERRS
RRLGCSWEFIPVDDGWGERPL
SEQ ID NO: 39
(Modified BoNT/A1 (N1188R, D1213R, G1215R, N1216R, N1242R, N1243R,
S1274R, T1277R, M1144V) Polypeptide Sequence)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESNHLIDLSRYASKINI
GSKVNFDPIDKNQIQLFNLESSKIEVILKNAIVYNSMYENFSTSFWIRIPKYFNSISLNNEYTIINCMENNSGWK
VSLNYGEIIWTLQDTQEIKQRVVFKYSQMINISDYINRWIFVTITNNRLNNSKIYINGRLIDQKPISNLGNIHAS
NNIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVVTTNIYLNSSLYRGTKFIIKKYASGNKDNIVRNNDRVYINVVVKRKEYRLATNASQA
GVEKILSALEIPRVRRLSQVVVMKSKNDQGITNKCKMNLQDRRGNDIGFIGFHQENNIAKLVASNWYNRQIERRS
RRLGCSWEFIPVDDGWGERPL
SEQ ID NO: 40
(Modified BoNT/A1 (N1188R, D1213R, G1215R, N1216R, N1242R, N1243R,
S1274R, T1277R, M1144G) Polypeptide Sequence)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESNHLIDLSRYASKINI
GSKVNFDPIDKNQIQLFNLESSKIEVILKNAIVYNSMYENFSTSFWIRIPKYFNSISLNNEYTIINCMENNSGWK
VSLNYGEIIWTLQDTQEIKQRVVFKYSQMINISDYINRWIFVTITNNRLNNSKIYINGRLIDQKPISNLGNIHAS
NNIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVGTTNIYLNSSLYRGTKFIIKKYASGNKDNIVRNNDRVYINVVVKRKEYRLATNASQA
GVEKILSALEIPRVRRLSQVVVMKSKNDQGITNKCKMNLQDRRGNDIGFIGFHQFNNIAKLVASNWYNRQIERRS
RRLGCSWEFIPVDDGWGERPL
SEQ ID NO: 41
(Modified BoNT/A1 (N1188R, D1213R, G1215R, N1216R, N1242R, N1243R,
S1274R, T1277R, M1144L) Polypeptide Sequence)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESNHLIDLSRYASKINI
GSKVNFDPIDKNQIQLFNLESSKIEVILKNAIVYNSMYENFSTSFWIRIPKYFNSISLNNEYTIINCMENNSGWK
VSLNYGEIIWTLQDTQEIKQRVVFKYSQMINISDYINRWIFVTITNNRLNNSKIYINGRLIDQKPISNLGNIHAS
NNIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVLTTNIYLNSSLYRGTKFIIKKYASGNKDNIVRNNDRVYINVVVKRKEYRLATNASQA
GVEKILSALEIPRVRRLSQVVVMKSKNDQGITNKCKMNLQDRRGNDIGFIGFHQFNNIAKLVASNWYNRQIERRS
RRLGCSWEFIPVDDGWGERPL
SEQ ID NO: 42
(Modified BoNT/A1 (N1188R, D1213R, G1215R, N1216R, N1242R, N1243R,
S1274R, T1277R, M1144T) Polypeptide Sequence)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESNHLIDLSRYASKINI
GSKVNFDPIDKNQIQLFNLESSKIEVILKNAIVYNSMYENFSTSFWIRIPKYFNSISLNNEYTIINCMENNSGWK
VSLNYGEIIWTLQDTQEIKQRVVFKYSQMINISDYINRWIFVTITNNRLNNSKIYINGRLIDQKPISNLGNIHAS
NNIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVTTTNIYLNSSLYRGTKFIIKKYASGNKDNIVRNNDRVYINVVVKRKEYRLATNASQA
GVEKILSALEIPRVRRLSQVVVMKSKNDQGITNKCKMNLQDRRGNDIGFIGFHQFNNIAKLVASNWYNRQIERRS
RRLGCSWEFIPVDDGWGERPL
SEQ ID NO: 43
(Modified BoNT/A1 (N1188R, D1213R, G1215R, N1216R, N1242R, N1243R,
S1274R, T1277R, M1144A) Polypeptide Sequence)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTENFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESNHLIDLSRYASKINI
GSKVNFDPIDKNQIQLFNLESSKIEVILKNAIVYNSMYENFSTSFWIRIPKYFNSISLNNEYTIINCMENNSGWK
VSLNYGEIIWTLQDTQEIKQRVVFKYSQMINISDYINRWIFVTITNNRLNNSKIYINGRLIDQKPISNLGNIHAS
NNIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVATTNIYLNSSLYRGTKFIIKKYASGNKDNIVRNNDRVYINVVVKRKEYRLATNASQA
GVEKILSALEIPRVRRLSQVVVMKSKNDQGITNKCKMNLQDRRGNDIGFIGFHQFNNIAKLVASNWYNRQIERRS
RRLGCSWEFIPVDDGWGERPL
SEQ ID NO: 44
(Modified BoNT/A1 (N1188R, D1213R, G1215R, N1216R, N1242R, N1243R,
S1274R, T1277R, M11441) Polypeptide Sequence)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESNHLIDLSRYASKINI
GSKVNFDPIDKNQIQLFNLESSKIEVILKNAIVYNSMYENFSTSFWIRIPKYFNSISLNNEYTIINCMENNSGWK
VSLNYGEIIWTLQDTQEIKQRVVFKYSQMINISDYINRWIFVTITNNRLNNSKIYINGRLIDQKPISNLGNIHAS
NNIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVITTNIYLNSSLYRGTKFIIKKYASGNKDNIVRNNDRVYINVVVKRKEYRLATNASQA
GVEKILSALEIPRVRRLSQVVVMKSKNDQGITNKCKMNLQDRRGNDIGFIGFHQFNNIAKLVASNWYNRQIERRS
RRLGCSWEFIPVDDGWGERPL
SEQ ID NO: 45
(Modified BoNT/A1 (N1188R, D1213R, G1215R, N1216R, N1242R, N1243R,
S1274R, T1277R, M1144 Deletion) Polypeptide Sequence)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTINKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESNHLIDLSRYASKINI
GSKVNFDPIDKNQIQLFNLESSKIEVILKNAIVYNSMYENFSTSFWIRIPKYFNSISLNNEYTIINCMENNSGWK
VSLNYGEIIWTLQDTQEIKQRVVFKYSQMINISDYINRWIFVTITNNRLNNSKIYINGRLIDQKPISNLGNIHAS
NNIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVTTNIYLNSSLYRGTKFIIKKYASGNKDNIVRNNDRVYINVVVKRKEYRLATNASQAG
VEKILSALEIPRVRRLSQVVVMKSKNDQGITNKCKMNLQDRRGNDIGFIGFHQFNNIAKLVASNWYNRQIERRSR
RLGCSWEFIPVDDGWGERPL
SEQ ID NO: 46
(BoNT/A3 Polypeptide Sequence)
MPFVNKPFNYRDPGNGVDIAYIKIPNAGQMQPVKAFKIHEGVWVIPERDTFTNPEEGDLN
PPPEAKQVPVSYYDSTYLSTDNEKDNYLKGVIKLFDRIYSTGLGRMLLSFIVKGIPFWGG
STIDTELKVIDTNCINVIEPGGSYRSEELNLVITGPSADIIQFECKSFGHDVFNLTRNGY
GSTQYIRFSPDFTFGFEESLEVDTNPLLGAGTFATDPAVTLAHELIHAAHRLYGIAINPN
RVLKVKTNAYYEMSGLEVSFEELRTFGGNDTNFIDSLWQKKFSRDAYDNLQNIARILNEA
KTIVGTTTPLQYMKNIFIRKYFLSEDASGKISVNKAAFKEFYRVLTRGFTELEFVNPFKV
INRKTYLNFDKAVFRINIVPDENYTINEGFNLEGANSNGQNTEINSRNFTRLKNFTGLFE
FYKLLCVRGIIPFKTKSLDEGYNKALNYLCIKVNNWDLFFSPSEDNFTNDLDKVEEITAD
TNIEAAEENISSDLIQQYYLTFDFDNEPENISIENLSSDIIGQLEPMPNIERFPNGKKYE
LDKYTMFHYLRAQEFEHGDSRIILTNSAEEALLKPNVAYTFFSSKYVKKINKAVEAVIFL
SWAEELVYDFTDETNEVTTMDKIADITIIVPYIGPALNIGNMVSKGEFVEAILFTGVVAL
LEFIPEYSLPVFGTFAIVSYIANKVLTVQTINNALSKRNEKWDEVYKYTVTNWLAKVNTQ
IDLIREKMKKALENQAEATRAIINYQYNQYTEEEKNNINFNIDDLSSKLNRSINRAMINI
NKFLDQCSVSYLMNSMIPYAVKRLKDFDASVRDVLLKYIYDNRGTLILQVDRLKDEVNNT
LSADIPFQLSKYVNDKKLLSTFTEYIKNIVNTSILSIVYKKDDLIDLSRYGAKINIGDRV
YYDSIDKNQIKLINLESSTIEVILKNAIVYNSMYENFSTSFWIKIPKYFSKINLNNEYTI
INCIENNSGWKVSLNYGEIIWTLQDNKQNIQRVVFKYSQMVNISDYINRWMFVTITNNRL
TKSKIYINGRLIDQKPISNLGNIHASNKIMFKLDGCRDPRRYIMIKYFNLFDKELNEKEI
KDLYDSQSNPGILKDFWGNYLQYDKPYYMLNLFDPNKYVDVNNIGIRGYMYLKGPRGSVM
TTNIYLNSTLYMGTKFIIKKYASGNEDNIVRNNDRVYINVVVKNKEYRLATNASQAGVEK
ILSALEIPDVGNLSQVVVMKSKDDQGIRNKCKMNLQDNNGNDIGFVGFHLYDNIAKLVAS
NWYNRQVGKASRTFGCSWEFIPVDDGWGESSL
SEQ ID NO: 47
(Modified BoNT/A3 (M1140V) Polypeptide Sequence)
MPFVNKPFNYRDPGNGVDIAYIKIPNAGQMQPVKAFKIHEGVWVIPERDTFTNPEEGDLN
PPPEAKQVPVSYYDSTYLSTDNEKDNYLKGVIKLFDRIYSTGLGRMLLSFIVKGIPFWGG
STIDTELKVIDTNCINVIEPGGSYRSEELNLVITGPSADIIQFECKSFGHDVFNLTRNGY
GSTQYIRFSPDFTFGFEESLEVDTNPLLGAGTFATDPAVTLAHELIHAAHRLYGIAINPN
RVLKVKTNAYYEMSGLEVSFEELRTFGGNDTNFIDSLWQKKFSRDAYDNLQNIARILNEA
KTIVGTTTPLQYMKNIFIRKYFLSEDASGKISVNKAAFKEFYRVLTRGFTELEFVNPFKV
INRKTYLNFDKAVFRINIVPDENYTINEGFNLEGANSNGQNTEINSRNFTRLKNFTGLFE
FYKLLCVRGIIPFKTKSLDEGYNKALNYLCIKVNNWDLFFSPSEDNFTNDLDKVEEITAD
TNIEAAEENISSDLIQQYYLTFDFDNEPENISIENLSSDIIGQLEPMPNIERFPNGKKYE
LDKYTMFHYLRAQEFEHGDSRIILTNSAEEALLKPNVAYTFFSSKYVKKINKAVEAVIFL
SWAEELVYDFTDETNEVTTMDKIADITIIVPYIGPALNIGNMVSKGEFVEAILFTGVVAL
LEFIPEYSLPVFGTFAIVSYIANKVLTVQTINNALSKRNEKWDEVYKYTVTNWLAKVNTQ
IDLIREKMKKALENQAEATRAIINYQYNQYTEEEKNNINFNIDDLSSKLNRSINRAMINI
NKFLDQCSVSYLMNSMIPYAVKRLKDFDASVRDVLLKYIYDNRGTLILQVDRLKDEVNNT
LSADIPFQLSKYVNDKKLLSTFTEYIKNIVNTSILSIVYKKDDLIDLSRYGAKINIGDRV
YYDSIDKNQIKLINLESSTIEVILKNAIVYNSMYENFSTSFWIKIPKYFSKINLNNEYTI
INCIENNSGWKVSLNYGEIIWTLQDNKQNIQRVVFKYSQMVNISDYINRWMFVTITNNRL
TKSKIYINGRLIDQKPISNLGNIHASNKIMFKLDGCRDPRRYIMIKYFNLFDKELNEKEI
KDLYDSQSNPGILKDFWGNYLQYDKPYYMLNLFDPNKYVDVNNIGIRGYMYLKGPRGSVV
TTNIYLNSTLYMGTKFIIKKYASGNEDNIVRNNDRVYINVVVKNKEYRLATNASQAGVEK
ILSALEIPDVGNLSQVVVMKSKDDQGIRNKCKMNLQDNNGNDIGFVGFHLYDNIAKLVAS
NWYNRQVGKASRTFGCSWEFIPVDDGWGESSL
SEQ ID NO: 48
(Modified BoNT/A3 (M1140G) Polypeptide Sequence)
MPFVNKPFNYRDPGNGVDIAYIKIPNAGQMQPVKAFKIHEGVWVIPERDTFTNPEEGDLN
PPPEAKQVPVSYYDSTYLSTDNEKDNYLKGVIKLFDRIYSTGLGRMLLSFIVKGIPFWGG
STIDTELKVIDTNCINVIEPGGSYRSEELNLVITGPSADIIQFECKSFGHDVFNLTRNGY
GSTQYIRFSPDFTFGFEESLEVDTNPLLGAGTFATDPAVTLAHELIHAAHRLYGIAINPN
RVLKVKTNAYYEMSGLEVSFEELRTFGGNDTNFIDSLWQKKFSRDAYDNLQNIARILNEA
KTIVGTTTPLQYMKNIFIRKYFLSEDASGKISVNKAAFKEFYRVLTRGFTELEFVNPFKV
INRKTYLNFDKAVFRINIVPDENYTINEGFNLEGANSNGQNTEINSRNFTRLKNFTGLFE
FYKLLCVRGIIPFKTKSLDEGYNKALNYLCIKVNNWDLFFSPSEDNFTNDLDKVEEITAD
TNIEAAEENISSDLIQQYYLTFDFDNEPENISIENLSSDIIGQLEPMPNIERFPNGKKYE
LDKYTMFHYLRAQEFEHGDSRIILTNSAEEALLKPNVAYTFFSSKYVKKINKAVEAVIFL
SWAEELVYDFTDETNEVTTMDKIADITIIVPYIGPALNIGNMVSKGEFVEAILFTGVVAL
LEFIPEYSLPVFGTFAIVSYIANKVLTVQTINNALSKRNEKWDEVYKYTVTNWLAKVNTQ
IDLIREKMKKALENQAEATRAIINYQYNQYTEEEKNNINFNIDDLSSKLNRSINRAMINI
NKFLDQCSVSYLMNSMIPYAVKRLKDFDASVRDVLLKYIYDNRGTLILQVDRLKDEVNNT
LSADIPFQLSKYVNDKKLLSTFTEYIKNIVNTSILSIVYKKDDLIDLSRYGAKINIGDRV
YYDSIDKNQIKLINLESSTIEVILKNAIVYNSMYENFSTSFWIKIPKYFSKINLNNEYTI
INCIENNSGWKVSLNYGEIIWTLQDNKQNIQRVVFKYSQMVNISDYINRWMFVTITNNRL
TKSKIYINGRLIDQKPISNLGNIHASNKIMFKLDGCRDPRRYIMIKYFNLFDKELNEKEI
KDLYDSQSNPGILKDFWGNYLQYDKPYYMLNLFDPNKYVDVNNIGIRGYMYLKGPRGSVG
TTNIYLNSTLYMGTKFIIKKYASGNEDNIVRNNDRVYINVVVKNKEYRLATNASQAGVEK
ILSALEIPDVGNLSQVVVMKSKDDQGIRNKCKMNLQDNNGNDIGFVGFHLYDNIAKLVAS
NWYNRQVGKASRTFGCSWEFIPVDDGWGESSL
SEQ ID NO: 49
(Modified BoNT/A3 (M1140L) Polypeptide Sequence)
MPFVNKPFNYRDPGNGVDIAYIKIPNAGQMQPVKAFKIHEGVWVIPERDTFTNPEEGDLN
PPPEAKQVPVSYYDSTYLSTDNEKDNYLKGVIKLFDRIYSTGLGRMLLSFIVKGIPFWGG
STIDTELKVIDTNCINVIEPGGSYRSEELNLVITGPSADIIQFECKSFGHDVFNLTRNGY
GSTQYIRFSPDFTFGFEESLEVDTNPLLGAGTFATDPAVTLAHELIHAAHRLYGIAINPN
RVLKVKTNAYYEMSGLEVSFEELRTFGGNDTNFIDSLWQKKFSRDAYDNLQNIARILNEA
KTIVGTTTPLQYMKNIFIRKYFLSEDASGKISVNKAAFKEFYRVLTRGFTELEFVNPFKV
INRKTYLNFDKAVFRINIVPDENYTINEGFNLEGANSNGQNTEINSRNFTRLKNFTGLFE
FYKLLCVRGIIPFKTKSLDEGYNKALNYLCIKVNNWDLFFSPSEDNFTNDLDKVEEITAD
TNIEAAEENISSDLIQQYYLTFDFDNEPENISIENLSSDIIGQLEPMPNIERFPNGKKYE
LDKYTMFHYLRAQEFEHGDSRIILTNSAEEALLKPNVAYTFFSSKYVKKINKAVEAVIFL
SWAEELVYDFTDETNEVTTMDKIADITIIVPYIGPALNIGNMVSKGEFVEAILFTGVVAL
LEFIPEYSLPVFGTFAIVSYIANKVLTVQTINNALSKRNEKWDEVYKYTVTNWLAKVNTQ
IDLIREKMKKALENQAEATRAIINYQYNQYTEEEKNNINFNIDDLSSKLNRSINRAMINI
NKFLDQCSVSYLMNSMIPYAVKRLKDFDASVRDVLLKYIYDNRGTLILQVDRLKDEVNNT
LSADIPFQLSKYVNDKKLLSTFTEYIKNIVNTSILSIVYKKDDLIDLSRYGAKINIGDRV
YYDSIDKNQIKLINLESSTIEVILKNAIVYNSMYENFSTSFWIKIPKYFSKINLNNEYTI
INCIENNSGWKVSLNYGEIIWTLQDNKQNIQRVVFKYSQMVNISDYINRWMFVTITNNRL
TKSKIYINGRLIDQKPISNLGNIHASNKIMFKLDGCRDPRRYIMIKYFNLFDKELNEKEI
KDLYDSQSNPGILKDFWGNYLQYDKPYYMLNLFDPNKYVDVNNIGIRGYMYLKGPRGSVL
TTNIYLNSTLYMGTKFIIKKYASGNEDNIVRNNDRVYINVVVKNKEYRLATNASQAGVEK
ILSALEIPDVGNLSQVVVMKSKDDQGIRNKCKMNLQDNNGNDIGFVGFHLYDNIAKLVAS
NWYNRQVGKASRTFGCSWEFIPVDDGWGESSL
SEQ ID NO: 50
(Modified BoNT/A3 (M1140T) Polypeptide Sequence)
MPFVNKPFNYRDPGNGVDIAYIKIPNAGQMQPVKAFKIHEGVWVIPERDTFTNPEEGDLN
PPPEAKQVPVSYYDSTYLSTDNEKDNYLKGVIKLFDRIYSTGLGRMLLSFIVKGIPFWGG
STIDTELKVIDTNCINVIEPGGSYRSEELNLVITGPSADIIQFECKSFGHDVFNLTRNGY
GSTQYIRFSPDFTFGFEESLEVDTNPLLGAGTFATDPAVTLAHELIHAAHRLYGIAINPN
RVLKVKTNAYYEMSGLEVSFEELRTFGGNDTNFIDSLWQKKFSRDAYDNLQNIARILNEA
KTIVGTTTPLQYMKNIFIRKYFLSEDASGKISVNKAAFKEFYRVLTRGFTELEFVNPFKV
INRKTYLNFDKAVFRINIVPDENYTINEGFNLEGANSNGQNTEINSRNFTRLKNFTGLFE
FYKLLCVRGIIPFKTKSLDEGYNKALNYLCIKVNNWDLFFSPSEDNFTNDLDKVEEITAD
TNIEAAEENISSDLIQQYYLTFDFDNEPENISIENLSSDIIGQLEPMPNIERFPNGKKYE
LDKYTMFHYLRAQEFEHGDSRIILTNSAEEALLKPNVAYTFFSSKYVKKINKAVEAVIFL
SWAEELVYDFTDETNEVTTMDKIADITIIVPYIGPALNIGNMVSKGEFVEAILFTGVVAL
LEFIPEYSLPVFGTFAIVSYIANKVLTVQTINNALSKRNEKWDEVYKYTVTNWLAKVNTQ
IDLIREKMKKALENQAEATRAIINYQYNQYTEEEKNNINFNIDDLSSKLNRSINRAMINI
NKFLDQCSVSYLMNSMIPYAVKRLKDFDASVRDVLLKYIYDNRGTLILQVDRLKDEVNNT
LSADIPFQLSKYVNDKKLLSTFTEYIKNIVNTSILSIVYKKDDLIDLSRYGAKINIGDRV
YYDSIDKNQIKLINLESSTIEVILKNAIVYNSMYENFSTSFWIKIPKYFSKINLNNEYTI
INCIENNSGWKVSLNYGEIIWTLQDNKQNIQRVVFKYSQMVNISDYINRWMFVTITNNRL
TKSKIYINGRLIDQKPISNLGNIHASNKIMFKLDGCRDPRRYIMIKYFNLFDKELNEKEI
KDLYDSQSNPGILKDFWGNYLQYDKPYYMLNLFDPNKYVDVNNIGIRGYMYLKGPRGSVT
TTNIYLNSTLYMGTKFIIKKYASGNEDNIVRNNDRVYINVVVKNKEYRLATNASQAGVEK
ILSALEIPDVGNLSQVVVMKSKDDQGIRNKCKMNLQDNNGNDIGFVGFHLYDNIAKLVAS
NWYNRQVGKASRTFGCSWEFIPVDDGWGESSL
SEQ ID NO: 51
(Modified BoNT/A3 (M1140A) Polypeptide Sequence)
MPFVNKPFNYRDPGNGVDIAYIKIPNAGQMQPVKAFKIHEGVWVIPERDTFTNPEEGDLN
PPPEAKQVPVSYYDSTYLSTDNEKDNYLKGVIKLFDRIYSTGLGRMLLSFIVKGIPFWGG
STIDTELKVIDTNCINVIEPGGSYRSEELNLVITGPSADIIQFECKSFGHDVFNLTRNGY
GSTQYIRFSPDFTFGFEESLEVDTNPLLGAGTFATDPAVTLAHELIHAAHRLYGIAINPN
RVLKVKTNAYYEMSGLEVSFEELRTFGGNDTNFIDSLWQKKFSRDAYDNLQNIARILNEA
KTIVGTTTPLQYMKNIFIRKYFLSEDASGKISVNKAAFKEFYRVLTRGFTELEFVNPFKV
INRKTYLNFDKAVFRINIVPDENYTINEGFNLEGANSNGQNTEINSRNFTRLKNFTGLFE
FYKLLCVRGIIPFKTKSLDEGYNKALNYLCIKVNNWDLFFSPSEDNFTNDLDKVEEITAD
TNIEAAEENISSDLIQQYYLTFDFDNEPENISIENLSSDIIGQLEPMPNIERFPNGKKYE
LDKYTMFHYLRAQEFEHGDSRIILTNSAEEALLKPNVAYTFFSSKYVKKINKAVEAVIFL
SWAEELVYDFTDETNEVTTMDKIADITIIVPYIGPALNIGNMVSKGEFVEAILFTGVVAL
LEFIPEYSLPVFGTFAIVSYIANKVLTVQTINNALSKRNEKWDEVYKYTVTNWLAKVNTQ
IDLIREKMKKALENQAEATRAIINYQYNQYTEEEKNNINFNIDDLSSKLNRSINRAMINI
NKFLDQCSVSYLMNSMIPYAVKRLKDFDASVRDVLLKYIYDNRGTLILQVDRLKDEVNNT
LSADIPFQLSKYVNDKKLLSTFTEYIKNIVNTSILSIVYKKDDLIDLSRYGAKINIGDRV
YYDSIDKNQIKLINLESSTIEVILKNAIVYNSMYENFSTSFWIKIPKYFSKINLNNEYTI
INCIENNSGWKVSLNYGEIIWTLQDNKQNIQRVVFKYSQMVNISDYINRWMFVTITNNRL
TKSKIYINGRLIDQKPISNLGNIHASNKIMFKLDGCRDPRRYIMIKYFNLFDKELNEKEI
KDLYDSQSNPGILKDFWGNYLQYDKPYYMLNLFDPNKYVDVNNIGIRGYMYLKGPRGSVA
TTNIYLNSTLYMGTKFIIKKYASGNEDNIVRNNDRVYINVVVKNKEYRLATNASQAGVEK
ILSALEIPDVGNLSQVVVMKSKDDQGIRNKCKMNLQDNNGNDIGFVGFHLYDNIAKLVAS
NWYNRQVGKASRTFGCSWEFIPVDDGWGESSL
SEQ ID NO: 52
(Modified BoNT/A3 (M11401) Polypeptide Sequence)
MPFVNKPFNYRDPGNGVDIAYIKIPNAGQMQPVKAFKIHEGVWVIPERDTFTNPEEGDLN
PPPEAKQVPVSYYDSTYLSTDNEKDNYLKGVIKLFDRIYSTGLGRMLLSFIVKGIPFWGG
STIDTELKVIDTNCINVIEPGGSYRSEELNLVITGPSADIIQFECKSFGHDVFNLTRNGY
GSTQYIRFSPDFTFGFEESLEVDTNPLLGAGTFATDPAVTLAHELIHAAHRLYGIAINPN
RVLKVKTNAYYEMSGLEVSFEELRTFGGNDTNFIDSLWQKKFSRDAYDNLQNIARILNEA
KTIVGTTTPLQYMKNIFIRKYFLSEDASGKISVNKAAFKEFYRVLTRGFTELEFVNPFKV
INRKTYLNFDKAVFRINIVPDENYTINEGFNLEGANSNGQNTEINSRNFTRLKNFTGLFE
FYKLLCVRGIIPFKTKSLDEGYNKALNYLCIKVNNWDLFFSPSEDNFTNDLDKVEEITAD
TNIEAAEENISSDLIQQYYLTFDFDNEPENISIENLSSDIIGQLEPMPNIERFPNGKKYE
LDKYTMFHYLRAQEFEHGDSRIILTNSAEEALLKPNVAYTFFSSKYVKKINKAVEAVIFL
SWAEELVYDFTDETNEVTTMDKIADITIIVPYIGPALNIGNMVSKGEFVEAILFTGVVAL
LEFIPEYSLPVFGTFAIVSYIANKVLTVQTINNALSKRNEKWDEVYKYTVTNWLAKVNTQ
IDLIREKMKKALENQAEATRAIINYQYNQYTEEEKNNINFNIDDLSSKLNRSINRAMINI
NKFLDQCSVSYLMNSMIPYAVKRLKDFDASVRDVLLKYIYDNRGTLILQVDRLKDEVNNT
LSADIPFQLSKYVNDKKLLSTFTEYIKNIVNTSILSIVYKKDDLIDLSRYGAKINIGDRV
YYDSIDKNQIKLINLESSTIEVILKNAIVYNSMYENFSTSFWIKIPKYFSKINLNNEYTI
INCIENNSGWKVSLNYGEIIWTLQDNKQNIQRVVFKYSQMVNISDYINRWMFVTITNNRL
TKSKIYINGRLIDQKPISNLGNIHASNKIMFKLDGCRDPRRYIMIKYFNLFDKELNEKEI
KDLYDSQSNPGILKDFWGNYLQYDKPYYMLNLFDPNKYVDVNNIGIRGYMYLKGPRGSVI
TTNIYLNSTLYMGTKFIIKKYASGNEDNIVRNNDRVYINVVVKNKEYRLATNASQAGVEK
ILSALEIPDVGNLSQVVVMKSKDDQGIRNKCKMNLQDNNGNDIGFVGFHLYDNIAKLVAS
NWYNRQVGKASRTFGCSWEFIPVDDGWGESSL
SEQ ID NO: 53
(Modified BoNT/A3 (M1140 Deletion) Polypeptide Sequence)
MPFVNKPFNYRDPGNGVDIAYIKIPNAGQMQPVKAFKIHEGVWVIPERDTFTNPEEGDLN
PPPEAKQVPVSYYDSTYLSTDNEKDNYLKGVIKLFDRIYSTGLGRMLLSFIVKGIPFWGG
STIDTELKVIDTNCINVIEPGGSYRSEELNLVITGPSADIIQFECKSFGHDVFNLTRNGY
GSTQYIRFSPDFTFGFEESLEVDTNPLLGAGTFATDPAVTLAHELIHAAHRLYGIAINPN
RVLKVKTNAYYEMSGLEVSFEELRTFGGNDTNFIDSLWQKKFSRDAYDNLQNIARILNEA
KTIVGTTTPLQYMKNIFIRKYFLSEDASGKISVNKAAFKEFYRVLTRGFTELEFVNPFKV
INRKTYLNFDKAVFRINIVPDENYTINEGFNLEGANSNGQNTEINSRNFTRLKNFTGLFE
FYKLLCVRGIIPFKTKSLDEGYNKALNYLCIKVNNWDLFFSPSEDNFTNDLDKVEEITAD
TNIEAAEENISSDLIQQYYLTFDFDNEPENISIENLSSDIIGQLEPMPNIERFPNGKKYE
LDKYTMFHYLRAQEFEHGDSRIILTNSAEEALLKPNVAYTFFSSKYVKKINKAVEAVIFL
SWAEELVYDFTDETNEVTTMDKIADITIIVPYIGPALNIGNMVSKGEFVEAILFTGVVAL
LEFIPEYSLPVFGTFAIVSYIANKVLTVQTINNALSKRNEKWDEVYKYTVTNWLAKVNTQ
IDLIREKMKKALENQAEATRAIINYQYNQYTEEEKNNINFNIDDLSSKLNRSINRAMINI
NKFLDQCSVSYLMNSMIPYAVKRLKDFDASVRDVLLKYIYDNRGTLILQVDRLKDEVNNT
LSADIPFQLSKYVNDKKLLSTFTEYIKNIVNTSILSIVYKKDDLIDLSRYGAKINIGDRV
YYDSIDKNQIKLINLESSTIEVILKNAIVYNSMYENFSTSFWIKIPKYFSKINLNNEYTI
INCIENNSGWKVSLNYGEIIWTLQDNKQNIQRVVFKYSQMVNISDYINRWMFVTITNNRL
TKSKIYINGRLIDQKPISNLGNIHASNKIMFKLDGCRDPRRYIMIKYFNLFDKELNEKEI
KDLYDSQSNPGILKDFWGNYLQYDKPYYMLNLFDPNKYVDVNNIGIRGYMYLKGPRGSV
TTNIYLNSTLYMGTKFIIKKYASGNEDNIVRNNDRVYINVVVKNKEYRLATNASQAGVEK
ILSALEIPDVGNLSQVVVMKSKDDQGIRNKCKMNLQDNNGNDIGFVGFHLYDNIAKLVAS
NWYNRQVGKASRTFGCSWEFIPVDDGWGESSL
SEQ ID NO: 54
(BoNT/A4 Polypeptide Sequence)
MPLVNQQINYYDPVNGVDIAYIKIPNAGKMQPVKAFKIHNKVWVIPERDIFTNPEEVDLN
PPPEAKQVPISYYDSAYLSTDNEKDNYLKGVIKLFERIYSTDLGRMLLISIVRGIPFWGG
GKIDTELKVIDTNCINIIQLDDSYRSEELNLAIIGPSANIIESQCSSFRDDVLNLTRNGY
GSTQYIRFSPDFTVGFEESLEVDTNPLLGAGKFAQDPAVALAHELIHAEHRLYGIAINTN
RVFKVNTNAYYEMAGLEVSLEELITFGGNDAKFIDSLQKKEFSLYYYNKFKDIASTLNKA
KSIVGTTASLQYMKNVFKEKYLLSEDATGKFLVDRLKFDELYKLLTEIYTEDNFVKFFKV
LNRKTYLNFDKAVFKINIVPDVNYTIHDGFNLRNTNLAANENGQNIEINNKNFDKLKNFT
GLFEFYKLLCVRGIITSKTKSLDEGYNKALNELCIKVNNWDLFFSPSEDNFTNDLDKVEE
ITSDTNIEAAEENISLDLIQQYYLNFNFDNEPENTSIENLSSDIIGQLEPMPNIERFPNG
KKYELNKYTMFHYLRAQEFKHSNSRIILTNSAKEALLKPNIVYTFFSSKYIKAINKAVEA
VTFVNWIENLVYDFTDETNEVSTMDKIADITIVIPYIGPALNIGNMIYKGEFVEAIIFSG
AVILLEIVPEIALPVLGTFALVSYVSNKVLTVQTIDNALSKRNEKWDEVYKYIVTNWLAI
VNTQINLIREKMKKALENQAEATKAIINYQYNQYTEEEKNNINFNIDDLSSKLNESINSA
MININKFLDQCSVSYLMNSMIPYAVKRLKDFDASVRDVLLKYIYDNRGTLIGQVNRLKDK
VNNTLSADIPFQLSKYVDNKKLLSTFTEYIKNITNASILSIVYKDDDLIDLSRYGAEIYN
GDKVYYNSIDKNQIRLINLESSTIEVILKKAIVYNSMYENFSTSFWIRIPKYFNSISLNN
EYTIINCMENNSGWKVSLNYGEIIWTFQDTQEIKQRVVFKYSQMINISDYINRWIFVTIT
NNRITKSKIYINGRLIDQKPISNLGNIHASNKIMFKLDGCRDPHRYIVIKYFNLFDKELS
EKEIKDLYDNQSNSGILKDFWGDYLQYDKSYYMLNLYDPNKYVDVNNVGIRGYMYLKGPR
DNVMTTNIYLNSSLYMGTKFIIKKYASGNKDNIVRNNDRVYINVVVKNKEYRLATNASQA
GVEKILSALEIPDVGNLSQVVVMKSKNDQGITNKCKMNLQDNNGNDIGFIGFHQFNNIAK
LVASNWYNRQIERSSRTLGCSWEFIPVDDGWRERPL
SEQ ID NO: 55
(Modified BoNT/A4 (M1144V) Polypeptide Sequence)
MPLVNQQINYYDPVNGVDIAYIKIPNAGKMQPVKAFKIHNKVWVIPERDIFTNPEEVDLN
PPPEAKQVPISYYDSAYLSTDNEKDNYLKGVIKLFERIYSTDLGRMLLISIVRGIPFWGG
GKIDTELKVIDTNCINIIQLDDSYRSEELNLAIIGPSANIIESQCSSFRDDVLNLTRNGY
GSTQYIRFSPDFTVGFEESLEVDTNPLLGAGKFAQDPAVALAHELIHAEHRLYGIAINTN
RVFKVNTNAYYEMAGLEVSLEELITFGGNDAKFIDSLQKKEFSLYYYNKFKDIASTLNKA
KSIVGTTASLQYMKNVFKEKYLLSEDATGKFLVDRLKFDELYKLLTEIYTEDNFVKFFKV
LNRKTYLNFDKAVFKINIVPDVNYTIHDGFNLRNTNLAANFNGQNIEINNKNFDKLKNFT
GLFEFYKLLCVRGIITSKTKSLDEGYNKALNELCIKVNNWDLFFSPSEDNFTNDLDKVEE
ITSDTNIEAAEENISLDLIQQYYLNFNFDNEPENTSIENLSSDIIGQLEPMPNIERFPNG
KKYELNKYTMFHYLRAQEFKHSNSRIILTNSAKEALLKPNIVYTFFSSKYIKAINKAVEA
VTFVNWIENLVYDFTDETNEVSTMDKIADITIVIPYIGPALNIGNMIYKGEFVEAIIFSG
AVILLEIVPEIALPVLGTFALVSYVSNKVLTVQTIDNALSKRNEKWDEVYKYIVTNWLAI
VNTQINLIREKMKKALENQAEATKAIINYQYNQYTEEEKNNINFNIDDLSSKLNESINSA
MININKFLDQCSVSYLMNSMIPYAVKRLKDFDASVRDVLLKYIYDNRGTLIGQVNRLKDK
VNNTLSADIPFQLSKYVDNKKLLSTFTEYIKNITNASILSIVYKDDDLIDLSRYGAEIYN
GDKVYYNSIDKNQIRLINLESSTIEVILKKAIVYNSMYENFSTSFWIRIPKYFNSISLNN
EYTIINCMENNSGWKVSLNYGEIIWTFQDTQEIKQRVVFKYSQMINISDYINRWIFVTIT
NNRITKSKIYINGRLIDQKPISNLGNIHASNKIMFKLDGCRDPHRYIVIKYENLEDKELS
EKEIKDLYDNQSNSGILKDFWGDYLQYDKSYYMLNLYDPNKYVDVNNVGIRGYMYLKGPR
DNVVTTNIYLNSSLYMGTKFIIKKYASGNKDNIVRNNDRVYINVVVKNKEYRLATNASQA
GVEKILSALEIPDVGNLSQVVVMKSKNDQGITNKCKMNLQDNNGNDIGFIGFHQFNNIAK
LVASNWYNRQIERSSRTLGCSWEFIPVDDGWRERPL
SEQ ID NO: 56
(Modified BoNT/A4 (M1144G) Polypeptide Sequence)
MPLVNQQINYYDPVNGVDIAYIKIPNAGKMQPVKAFKIHNKVWVIPERDIFTNPEEVDLN
PPPEAKQVPISYYDSAYLSTDNEKDNYLKGVIKLFERIYSTDLGRMLLISIVRGIPFWGG
GKIDTELKVIDTNCINIIQLDDSYRSEELNLAIIGPSANIIESQCSSERDDVLNLTRNGY
GSTQYIRFSPDFTVGFEESLEVDTNPLLGAGKFAQDPAVALAHELIHAEHRLYGIAINTN
RVFKVNTNAYYEMAGLEVSLEELITFGGNDAKFIDSLQKKEFSLYYYNKFKDIASTLNKA
KSIVGTTASLQYMKNVFKEKYLLSEDATGKFLVDRLKFDELYKLLTEIYTEDNFVKFFKV
LNRKTYLNFDKAVFKINIVPDVNYTIHDGFNLRNTNLAANFNGQNIEINNKNFDKLKNFT
GLFEFYKLLCVRGIITSKTKSLDEGYNKALNELCIKVNNWDLFFSPSEDNFTNDLDKVEE
ITSDTNIEAAEENISLDLIQQYYLNFNFDNEPENTSIENLSSDIIGQLEPMPNIERFPNG
KKYELNKYTMFHYLRAQEFKHSNSRIILTNSAKEALLKPNIVYTFFSSKYIKAINKAVEA
VTFVNWIENLVYDFTDETNEVSTMDKIADITIVIPYIGPALNIGNMIYKGEFVEAIIFSG
AVILLEIVPEIALPVLGTFALVSYVSNKVLTVQTIDNALSKRNEKWDEVYKYIVTNWLAI
VNTQINLIREKMKKALENQAEATKAIINYQYNQYTEEEKNNINFNIDDLSSKLNESINSA
MININKFLDQCSVSYLMNSMIPYAVKRLKDFDASVRDVLLKYIYDNRGTLIGQVNRLKDK
VNNTLSADIPFQLSKYVDNKKLLSTFTEYIKNITNASILSIVYKDDDLIDLSRYGAEIYN
GDKVYYNSIDKNQIRLINLESSTIEVILKKAIVYNSMYENFSTSFWIRIPKYFNSISLNN
EYTIINCMENNSGWKVSLNYGEIIWTFQDTQEIKQRVVFKYSQMINISDYINRWIFVTIT
NNRITKSKIYINGRLIDQKPISNLGNIHASNKIMFKLDGCRDPHRYIVIKYENLFDKELS
EKEIKDLYDNQSNSGILKDFWGDYLQYDKSYYMLNLYDPNKYVDVNNVGIRGYMYLKGPR
DNVGTTNIYLNSSLYMGTKFIIKKYASGNKDNIVRNNDRVYINVVVKNKEYRLATNASQA
GVEKILSALEIPDVGNLSQVVVMKSKNDQGITNKCKMNLQDNNGNDIGFIGFHQFNNIAK
LVASNWYNRQIERSSRTLGCSWEFIPVDDGWRERPL
SEQ ID NO: 57
(Modified BoNT/A4 (M1144L) Polypeptide Sequence)
MPLVNQQINYYDPVNGVDIAYIKIPNAGKMQPVKAFKIHNKVWVIPERDIFTNPEEVDLN
PPPEAKQVPISYYDSAYLSTDNEKDNYLKGVIKLFERIYSTDLGRMLLISIVRGIPFWGG
GKIDTELKVIDTNCINIIQLDDSYRSEELNLAIIGPSANIIESQCSSFRDDVLNLTRNGY
GSTQYIRFSPDFTVGFEESLEVDTNPLLGAGKFAQDPAVALAHELIHAEHRLYGIAINTN
RVFKVNTNAYYEMAGLEVSLEELITFGGNDAKFIDSLQKKEFSLYYYNKFKDIASTLNKA
KSIVGTTASLQYMKNVFKEKYLLSEDATGKFLVDRLKFDELYKLLTEIYTEDNFVKFFKV
LNRKTYLNFDKAVFKINIVPDVNYTIHDGFNLRNTNLAANFNGQNIEINNKNFDKLKNFT
GLFEFYKLLCVRGIITSKTKSLDEGYNKALNELCIKVNNWDLFFSPSEDNFTNDLDKVEE
ITSDTNIEAAEENISLDLIQQYYLNFNFDNEPENTSIENLSSDIIGQLEPMPNIERFPNG
KKYELNKYTMFHYLRAQEFKHSNSRIILTNSAKEALLKPNIVYTFFSSKYIKAINKAVEA
VTFVNWIENLVYDFTDETNEVSTMDKIADITIVIPYIGPALNIGNMIYKGEFVEAIIFSG
AVILLEIVPEIALPVLGTFALVSYVSNKVLTVQTIDNALSKRNEKWDEVYKYIVTNWLAI
VNTQINLIREKMKKALENQAEATKAIINYQYNQYTEEEKNNINFNIDDLSSKLNESINSA
MININKFLDQCSVSYLMNSMIPYAVKRLKDFDASVRDVLLKYIYDNRGTLIGQVNRLKDK
VNNTLSADIPFQLSKYVDNKKLLSTFTEYIKNITNASILSIVYKDDDLIDLSRYGAEIYN
GDKVYYNSIDKNQIRLINLESSTIEVILKKAIVYNSMYENFSTSFWIRIPKYFNSISLNN
EYTIINCMENNSGWKVSLNYGEIIWTFQDTQEIKQRVVFKYSQMINISDYINRWIFVTIT
NNRITKSKIYINGRLIDQKPISNLGNIHASNKIMFKLDGCRDPHRYIVIKYFNLFDKELS
EKEIKDLYDNQSNSGILKDFWGDYLQYDKSYYMLNLYDPNKYVDVNNVGIRGYMYLKGPR
DNVLTTNIYLNSSLYMGTKFIIKKYASGNKDNIVRNNDRVYINVVVKNKEYRLATNASQA
GVEKILSALEIPDVGNLSQVVVMKSKNDQGITNKCKMNLQDNNGNDIGFIGFHQFNNIAK
LVASNWYNRQIERSSRTLGCSWEFIPVDDGWRERPL
SEQ ID NO: 58
(Modified BoNT/A4 (M1144T) Polypeptide Sequence)
MPLVNQQINYYDPVNGVDIAYIKIPNAGKMQPVKAFKIHNKVWVIPERDIFTNPEEVDLN
PPPEAKQVPISYYDSAYLSTDNEKDNYLKGVIKLFERIYSTDLGRMLLISIVRGIPFWGG
GKIDTELKVIDTNCINIIQLDDSYRSEELNLAIIGPSANIIESQCSSFRDDVLNLTRNGY
GSTQYIRFSPDFTVGFEESLEVDTNPLLGAGKFAQDPAVALAHELIHAEHRLYGIAINTN
RVFKVNTNAYYEMAGLEVSLEELITFGGNDAKFIDSLQKKEFSLYYYNKFKDIASTINKA
KSIVGTTASLQYMKNVFKEKYLLSEDATGKFLVDRLKFDELYKLLTEIYTEDNFVKFFKV
LNRKTYLNFDKAVFKINIVPDVNYTIHDGFNLRNTNLAANFNGQNIEINNKNFDKLKNFT
GLFEFYKLLCVRGIITSKTKSLDEGYNKALNELCIKVNNWDLFFSPSEDNFTNDLDKVEE
ITSDTNIEAAEENISLDLIQQYYLNFNFDNEPENTSIENLSSDIIGQLEPMPNIERFPNG
KKYELNKYTMFHYLRAQEFKHSNSRIILTNSAKEALLKPNIVYTFFSSKYIKAINKAVEA
VTFVNWIENLVYDFTDETNEVSTMDKIADITIVIPYIGPALNIGNMIYKGEFVEAIIFSG
AVILLEIVPEIALPVLGTFALVSYVSNKVLTVQTIDNALSKRNEKWDEVYKYIVTNWLAI
VNTQINLIREKMKKALENQAEATKAIINYQYNQYTEEEKNNINFNIDDLSSKLNESINSA
MININKFLDQCSVSYLMNSMIPYAVKRLKDFDASVRDVLLKYIYDNRGTLIGQVNRLKDK
VNNTLSADIPFQLSKYVDNKKLLSTFTEYIKNITNASILSIVYKDDDLIDLSRYGAEIYN
GDKVYYNSIDKNQIRLINLESSTIEVILKKAIVYNSMYENFSTSFWIRIPKYFNSISLNN
EYTIINCMENNSGWKVSLNYGEIIWTFQDTQEIKQRVVFKYSQMINISDYINRWIFVTIT
NNRITKSKIYINGRLIDQKPISNLGNIHASNKIMFKLDGCRDPHRYIVIKYENLEDKELS
EKEIKDLYDNQSNSGILKDFWGDYLQYDKSYYMLNLYDPNKYVDVNNVGIRGYMYLKGPR
DNVTTTNIYLNSSLYMGTKFIIKKYASGNKDNIVRNNDRVYINVVVKNKEYRLATNASQA
GVEKILSALEIPDVGNLSQVVVMKSKNDQGITNKCKMNLQDNNGNDIGFIGFHQFNNIAK
LVASNWYNRQIERSSRTLGCSWEFIPVDDGWRERPL
SEQ ID NO: 59
(Modified BoNT/A4 (M1144A) Polypeptide Sequence)
MPLVNQQINYYDPVNGVDIAYIKIPNAGKMQPVKAFKIHNKVWVIPERDIFTNPEEVDLN
PPPEAKQVPISYYDSAYLSTDNEKDNYLKGVIKLFERIYSTDLGRMLLISIVRGIPFWGG
GKIDTELKVIDTNCINIIQLDDSYRSEELNLAIIGPSANIIESQCSSFRDDVLNLTRNGY
GSTQYIRFSPDFTVGFEESLEVDTNPLLGAGKFAQDPAVALAHELIHAEHRLYGIAINTN
RVFKVNTNAYYEMAGLEVSLEELITFGGNDAKFIDSLQKKEFSLYYYNKFKDIASTLNKA
KSIVGTTASLQYMKNVFKEKYLLSEDATGKFLVDRLKFDELYKLLTEIYTEDNFVKFFKV
LNRKTYLNFDKAVFKINIVPDVNYTIHDGFNLRNTNLAANFNGQNIEINNKNFDKLKNFT
GLFEFYKLLCVRGIITSKTKSLDEGYNKALNELCIKVNNWDLFFSPSEDNFTNDLDKVEE
ITSDTNIEAAEENISLDLIQQYYLNFNFDNEPENTSIENLSSDIIGQLEPMPNIERFPNG
KKYELNKYTMFHYLRAQEFKHSNSRIILTNSAKEALLKPNIVYTFFSSKYIKAINKAVEA
VTFVNWIENLVYDFTDETNEVSTMDKIADITIVIPYIGPALNIGNMIYKGEFVEAIIFSG
AVILLEIVPEIALPVLGTFALVSYVSNKVLTVQTIDNALSKRNEKWDEVYKYIVTNWLAI
VNTQINLIREKMKKALENQAEATKAIINYQYNQYTEEEKNNINFNIDDLSSKLNESINSA
MININKFLDQCSVSYLMNSMIPYAVKRLKDFDASVRDVLLKYIYDNRGTLIGQVNRLKDK
VNNTLSADIPFQLSKYVDNKKLLSTFTEYIKNITNASILSIVYKDDDLIDLSRYGAEIYN
GDKVYYNSIDKNQIRLINLESSTIEVILKKAIVYNSMYENFSTSFWIRIPKYFNSISLNN
EYTIINCMENNSGWKVSLNYGEIIWTFQDTQEIKQRVVFKYSQMINISDYINRWIFVTIT
NNRITKSKIYINGRLIDQKPISNLGNIHASNKIMFKLDGCRDPHRYIVIKYENLEDKELS
EKEIKDLYDNQSNSGILKDFWGDYLQYDKSYYMLNLYDPNKYVDVNNVGIRGYMYLKGPR
DNVATTNIYLNSSLYMGTKFIIKKYASGNKDNIVRNNDRVYINVVVKNKEYRLATNASQA
GVEKILSALEIPDVGNLSQVVVMKSKNDQGITNKCKMNLQDNNGNDIGFIGFHQFNNIAK
LVASNWYNRQIERSSRTLGCSWEFIPVDDGWRERPL
SEQ ID NO: 60
(Modified BoNT/A4 (M11441) Polypeptide Sequence)
MPLVNQQINYYDPVNGVDIAYIKIPNAGKMQPVKAFKIHNKVWVIPERDIFTNPEEVDLN
PPPEAKQVPISYYDSAYLSTDNEKDNYLKGVIKLFERIYSTDLGRMLLISIVRGIPFWGG
GKIDTELKVIDTNCINIIQLDDSYRSEELNLAIIGPSANIIESQCSSFRDDVLNLTRNGY
GSTQYIRFSPDFTVGFEESLEVDTNPLLGAGKFAQDPAVALAHELIHAEHRLYGIAINTN
RVFKVNTNAYYEMAGLEVSLEELITFGGNDAKFIDSLQKKEFSLYYYNKFKDIASTLNKA
KSIVGTTASLQYMKNVFKEKYLLSEDATGKFLVDRLKFDELYKLLTEIYTEDNFVKFFKV
LNRKTYLNFDKAVFKINIVPDVNYTIHDGFNLRNTNLAANENGQNIEINNKNFDKLKNFT
GLFEFYKLLCVRGIITSKTKSLDEGYNKALNELCIKVNNWDLFFSPSEDNFTNDLDKVEE
ITSDTNIEAAEENISLDLIQQYYLNFNFDNEPENTSIENLSSDIIGQLEPMPNIEREPNG
KKYELNKYTMFHYLRAQEFKHSNSRIILTNSAKEALLKPNIVYTFFSSKYIKAINKAVEA
VTFVNWIENLVYDFTDETNEVSTMDKIADITIVIPYIGPALNIGNMIYKGEFVEAIIFSG
AVILLEIVPEIALPVLGTFALVSYVSNKVLTVQTIDNALSKRNEKWDEVYKYIVTNWLAI
VNTQINLIREKMKKALENQAEATKAIINYQYNQYTEEEKNNINFNIDDLSSKINESINSA
MININKFLDQCSVSYLMNSMIPYAVKRLKDFDASVRDVLLKYIYDNRGTLIGQVNRLKDK
VNNTLSADIPFQLSKYVDNKKLLSTFTEYIKNITNASILSIVYKDDDLIDLSRYGAEIYN
GDKVYYNSIDKNQIRLINLESSTIEVILKKAIVYNSMYENFSTSFWIRIPKYFNSISLNN
EYTIINCMENNSGWKVSLNYGEIIWTFQDTQEIKQRVVFKYSQMINISDYINRWIFVTIT
NNRITKSKIYINGRLIDQKPISNLGNIHASNKIMFKLDGCRDPHRYIVIKYENLEDKELS
EKEIKDLYDNQSNSGILKDFWGDYLQYDKSYYMLNLYDPNKYVDVNNVGIRGYMYLKGPR
DNVITTNIYLNSSLYMGTKFIIKKYASGNKDNIVRNNDRVYINVVVKNKEYRLATNASQA
GVEKILSALEIPDVGNLSQVVVMKSKNDQGITNKCKMNLQDNNGNDIGFIGFHQFNNIAK
LVASNWYNRQIERSSRTLGCSWEFIPVDDGWRERPL
SEQ ID NO: 61
(Modified BoNT/A4 (M1144 Deletion) Polypeptide Sequence)
MPLVNQQINYYDPVNGVDIAYIKIPNAGKMQPVKAFKIHNKVWVIPERDIFTNPEEVDLN
PPPEAKQVPISYYDSAYLSTDNEKDNYLKGVIKLFERIYSTDLGRMLLISIVRGIPFWGG
GKIDTELKVIDTNCINIIQLDDSYRSEELNLAIIGPSANIIESQCSSFRDDVLNLTRNGY
GSTQYIRFSPDFTVGFEESLEVDTNPLLGAGKFAQDPAVALAHELIHAEHRLYGIAINTN
RVFKVNTNAYYEMAGLEVSLEELITFGGNDAKFIDSLQKKEFSLYYYNKFKDIASTLNKA
KSIVGTTASLQYMKNVFKEKYLLSEDATGKFLVDRLKFDELYKLLTEIYTEDNFVKFFKV
LNRKTYLNFDKAVFKINIVPDVNYTIHDGFNLRNTNLAANENGQNIEINNKNFDKLKNFT
GLFEFYKLLCVRGIITSKTKSLDEGYNKALNELCIKVNNWDLFFSPSEDNFTNDLDKVEE
ITSDTNIEAAEENISLDLIQQYYLNFNFDNEPENTSIENLSSDIIGQLEPMPNIEREPNG
KKYELNKYTMFHYLRAQEFKHSNSRIILTNSAKEALLKPNIVYTFFSSKYIKAINKAVEA
VTFVNWIENLVYDFTDETNEVSTMDKIADITIVIPYIGPALNIGNMIYKGEFVEAIIFSG
AVILLEIVPEIALPVLGTFALVSYVSNKVLTVQTIDNALSKRNEKWDEVYKYIVTNWLAI
VNTQINLIREKMKKALENQAEATKAIINYQYNQYTEEEKNNINFNIDDLSSKLNESINSA
MININKFLDQCSVSYLMNSMIPYAVKRLKDFDASVRDVLLKYIYDNRGTLIGQVNRLKDK
VNNTLSADIPFQLSKYVDNKKLLSTFTEYIKNITNASILSIVYKDDDLIDLSRYGAEIYN
GDKVYYNSIDKNQIRLINLESSTIEVILKKAIVYNSMYENFSTSFWIRIPKYFNSISLNN
EYTIINCMENNSGWKVSLNYGEIIWTFQDTQEIKQRVVFKYSQMINISDYINRWIFVTIT
NNRITKSKIYINGRLIDQKPISNLGNIHASNKIMFKLDGCRDPHRYIVIKYENLEDKELS
EKEIKDLYDNQSNSGILKDFWGDYLQYDKSYYMLNLYDPNKYVDVNNVGIRGYMYLKGPR
DNVTTNIYLNSSLYMGTKFIIKKYASGNKDNIVRNNDRVYINVVVKNKEYRLATNASQA
GVEKILSALEIPDVGNLSQVVVMKSKNDQGITNKCKMNLQDNNGNDIGFIGFHQFNNIAK
LVASNWYNRQIERSSRTLGCSWEFIPVDDGWRERPL
SEQ ID NO: 62
(BoNT/A1 H_{cc} domain Polypeptide Sequence)
YYMLNLYDPNKYVDVNNVGIRGYMYLKGPRGSVMTTNIYLNSSLYRGTKFIIKKYASGNKDNIVRNNDRVYINVV
VKNKEYRLATNASQAGVEKILSALEIPDVGNLSQVVVMKSKNDQGITNKCKMNLQDNNGNDIGFIGFHQFNNIAK
LVASNWYNRQIERSSRTLGCSWEFIPVDDGWGERPL
SEQ ID NO: 63
(Modified BoNT/A1 H_{cc} domain (M1144V) Polypeptide Sequence)
YYMLNLYDPNKYVDVNNVGIRGYMYLKGPRGSVVTTNIYLNSSLYRGTKFIIKKYASGNKDNIVRNNDRVYINVV
VKNKEYRLATNASQAGVEKILSALEIPDVGNLSQVVVMKSKNDQGITNKCKMNLQDNNGNDIGFIGFHQFNNIAK
LVASNWYNRQIERSSRTLGCSWEFIPVDDGWGERPL
SEQ ID NO: 64
(Modified BoNT/A1 H_{cc} domain (M1144G) Polypeptide Sequence)
YYMLNLYDPNKYVDVNNVGIRGYMYLKGPRGSVGTTNIYLNSSLYRGTKFIIKKYASGNKDNIVRNNDRVYINVV
VKNKEYRLATNASQAGVEKILSALEIPDVGNLSQVVVMKSKNDQGITNKCKMNLQDNNGNDIGFIGFHQFNNIAK
LVASNWYNRQIERSSRTLGCSWEFIPVDDGWGERPL
SEQ ID NO: 65
(Modified BoNT/A1 H_{cc} domain (M1144L) Polypeptide Sequence)
YYMLNLYDPNKYVDVNNVGIRGYMYLKGPRGSVLTTNIYLNSSLYRGTKFIIKKYASGNKDNIVRNNDRVYINVV
VKNKEYRLATNASQAGVEKILSALEIPDVGNLSQVVVMKSKNDQGITNKCKMNLQDNNGNDIGFIGFHQFNNIAK
LVASNWYNRQIERSSRTLGCSWEFIPVDDGWGERPL
SEQ ID NO: 66
(Modified BoNT/A1 H_{cc} domain (M1144T) Polypeptide Sequence)
YYMLNLYDPNKYVDVNNVGIRGYMYLKGPRGSVTTTNIYLNSSLYRGTKFIIKKYASGNKDNIVRNNDRVYINVV
VKNKEYRLATNASQAGVEKILSALEIPDVGNLSQVVVMKSKNDQGITNKCKMNLQDNNGNDIGFIGFHQFNNIAK
LVASNWYNRQIERSSRTLGCSWEFIPVDDGWGERPL
SEQ ID NO: 67
(Modified BoNT/A1 H_{cc} domain (M1144A) Polypeptide Sequence)
YYMLNLYDPNKYVDVNNVGIRGYMYLKGPRGSVATTNIYLNSSLYRGTKFIIKKYASGNKDNIVRNNDRVYINVV
VKNKEYRLATNASQAGVEKILSALEIPDVGNLSQVVVMKSKNDQGITNKCKMNLQDNNGNDIGFIGFHQFNNIAK
LVASNWYNRQIERSSRTLGCSWEFIPVDDGWGERPL
SEQ ID NO: 68
(Modified BoNT/A1 H_{cc} domain (M11441) Polypeptide Sequence)
YYMLNLYDPNKYVDVNNVGIRGYMYLKGPRGSVITTNIYLNSSLYRGTKFIIKKYASGNKDNIVRNNDRVYINVV
VKNKEYRLATNASQAGVEKILSALEIPDVGNLSQVVVMKSKNDQGITNKCKMNLQDNNGNDIGFIGFHQENNIAK
LVASNWYNRQIERSSRTLGCSWEFIPVDDGWGERPL
SEQ ID NO: 69
(Modified BoNT/A1 H_{cc} domain (M1144 Deletion) Polypeptide Sequence)
YYMLNLYDPNKYVDVNNVGIRGYMYLKGPRGSVTTNIYLNSSLYRGTKFIIKKYASGNKDNIVRNNDRVYINVVV
KNKEYRLATNASQAGVEKILSALEIPDVGNLSQVVVMKSKNDQGITNKCKMNLQDNNGNDIGFIGFHQFNNIAKL
VASNWYNRQIERSSRTLGCSWEFIPVDDGWGERPL
SEQ ID NO: 70
(BoNT/A1 H_{cc} domain (Q1229K) Polypeptide Sequence)
YYMLNLYDPNKYVDVNNVGIRGYMYLKGPRGSVMTTNIYLNSSLYRGTKFIIKKYASGNKDNIVRNNDRVYINVV
VKNKEYRLATNASQAGVEKILSALEIPDVGNLSQVVVMKSKNDKGITNKCKMNLQDNNGNDIGFIGFHQFNNIAK
LVASNWYNRQIERSSRTLGCSWEFIPVDDGWGERPL
SEQ ID NO: 71
(Modified BoNT/A1 H_{cc} domain (Q1229K, M1144V) Polypeptide Sequence)
YYMLNLYDPNKYVDVNNVGIRGYMYLKGPRGSVVTTNIYLNSSLYRGTKFIIKKYASGNKDNIVRNNDRVYINVV
VKNKEYRLATNASQAGVEKILSALEIPDVGNLSQVVVMKSKNDKGITNKCKMNLQDNNGNDIGFIGFHQFNNIAK
LVASNWYNRQIERSSRTLGCSWEFIPVDDGWGERPL
SEQ ID NO: 72
(Modified BoNT/A1 H_{cc} domain (Q1229K, M1144G) Polypeptide Sequence)
YYMLNLYDPNKYVDVNNVGIRGYMYLKGPRGSVGTTNIYLNSSLYRGTKFIIKKYASGNKDNIVRNNDRVYINVV
VKNKEYRLATNASQAGVEKILSALEIPDVGNLSQVVVMKSKNDKGITNKCKMNLQDNNGNDIGFIGFHQFNNIAK
LVASNWYNRQIERSSRTLGCSWEFIPVDDGWGERPL
SEQ ID NO: 73
(Modified BoNT/A1 H_{cc} domain (Q1229K, M1144L) Polypeptide Sequence)
YYMLNLYDPNKYVDVNNVGIRGYMYLKGPRGSVLTTNIYLNSSLYRGTKFIIKKYASGNKDNIVRNNDRVYINVV
VKNKEYRLATNASQAGVEKILSALEIPDVGNLSQVVVMKSKNDKGITNKCKMNLQDNNGNDIGFIGFHQFNNIAK
LVASNWYNRQIERSSRTLGCSWEFIPVDDGWGERPL
SEQ ID NO: 74
(Modified BoNT/A1 H_{cc} domain (Q1229K, M1144T) Polypeptide Sequence)
YYMLNLYDPNKYVDVNNVGIRGYMYLKGPRGSVTTTNIYLNSSLYRGTKFIIKKYASGNKDNIVRNNDRVYINVV
VKNKEYRLATNASQAGVEKILSALEIPDVGNLSQVVVMKSKNDKGITNKCKMNLQDNNGNDIGFIGFHQFNNIAK
LVASNWYNRQIERSSRTLGCSWEFIPVDDGWGERPL
SEQ ID NO: 75
(Modified BoNT/A1 H_{cc} domain (Q1229K, M1144A) Polypeptide Sequence)
YYMLNLYDPNKYVDVNNVGIRGYMYLKGPRGSVATTNIYLNSSLYRGTKFIIKKYASGNKDNIVRNNDRVYINVV
VKNKEYRLATNASQAGVEKILSALEIPDVGNLSQVVVMKSKNDKGITNKCKMNLQDNNGNDIGFIGFHQFNNIAK
LVASNWYNRQIERSSRTLGCSWEFIPVDDGWGERPL
SEQ ID NO: 76
(Modified BoNT/A1 H_{cc} domain (Q1229K, M11441) Polypeptide Sequence)
YYMLNLYDPNKYVDVNNVGIRGYMYLKGPRGSVITTNIYLNSSLYRGTKFIIKKYASGNKDNIVRNNDRVYINVV
VKNKEYRLATNASQAGVEKILSALEIPDVGNLSQVVVMKSKNDKGITNKCKMNLQDNNGNDIGFIGFHQFNNIAK
LVASNWYNRQIERSSRTLGCSWEFIPVDDGWGERPL
SEQ ID NO: 77
(Modified BoNT/A1 H_{cc} domain (Q1229K, M1144 Deletion) Polypeptide
Sequence)
YYMLNLYDPNKYVDVNNVGIRGYMYLKGPRGSVTTNIYLNSSLYRGTKFIIKKYASGNKDNIVRNNDRVYINVVV
KNKEYRLATNASQAGVEKILSALEIPDVGNLSQVVVMKSKNDKGITNKCKMNLQDNNGNDIGFIGFHQFNNIAKL
VASNWYNRQIERSSRTLGCSWEFIPVDDGWGERPL
SEQ ID NO: 78
(BoNT/A1 H_{cc} domain (N1188R, D1213R, G1215R, N1216R, N1242R,
N1243R, S1274R, T1277R) Polypeptide Sequence)
YYMLNLYDPNKYVDVNNVGIRGYMYLKGPRGSVMTTNIYLNSSLYRGTKFIIKKYASGNKDNIVRNNDRVYINVV
VKRKEYRLATNASQAGVEKILSALEIPRVRRLSQVVVMKSKNDQGITNKCKMNLQDRRGNDIGFIGFHQFNNIAK
LVASNWYNRQIERRSRRLGCSWEFIPVDDGWGERPL
SEQ ID NO: 79
(Modified BoNT/A1 H_{cc} domain (N1188R, D1213R, G1215R, N1216R,
N1242R, N1243R, S1274R, T1277R, M1144V) Polypeptide
YYMLNLYDPNKYVDVNNVGIRGYMYLKGPRGSVVTTNIYLNSSLYRGTKFIIKKYASGNKDNIVRNNDRVYINVV
VKRKEYRLATNASQAGVEKILSALEIPRVRRLSQVVVMKSKNDQGITNKCKMNLQDRRGNDIGFIGFHQFNNIAK
LVASNWYNRQIERRSRRLGCSWEFIPVDDGWGERPL
SEQ ID NO: 80
(Modified BoNT/A1 H_{cc} domain (N1188R, D1213R, G1215R, N1216R,
N1242R, N1243R, S1274R, T1277R, M1144G) Polypeptide Sequence)
YYMLNLYDPNKYVDVNNVGIRGYMYLKGPRGSVGTTNIYLNSSLYRGTKFIIKKYASGNKDNIVRNNDRVYINVV
VKRKEYRLATNASQAGVEKILSALEIPRVRRLSQVVVMKSKNDQGITNKCKMNLQDRRGNDIGFIGFHQFNNIAK
LVASNWYNRQIERRSRRLGCSWEFIPVDDGWGERPL
SEQ ID NO: 81
(Modified BoNT/A1 H_{cc} domain (N1188R, D1213R, G1215R, N1216R,
N1242R, N1243R, S1274R, T1277R, M1144L) Polypeptide Sequence)
YYMLNLYDPNKYVDVNNVGIRGYMYLKGPRGSVLTTNIYLNSSLYRGTKFIIKKYASGNKDNIVRNNDRVYINVV
VKRKEYRLATNASQAGVEKILSALEIPRVRRLSQVVVMKSKNDQGITNKCKMNLQDRRGNDIGFIGFHQFNNIAK
LVASNWYNRQIERRSRRLGCSWEFIPVDDGWGERPL
SEQ ID NO: 82
(Modified BoNT/A1 H_{cc} domain (N1188R, D1213R, G1215R, N1216R,
N1242R, N1243R, S1274R, T1277R, M1144T) Polypeptide Sequence)
YYMLNLYDPNKYVDVNNVGIRGYMYLKGPRGSVTTTNIYLNSSLYRGTKFIIKKYASGNKDNIVRNNDRVYINVV
VKRKEYRLATNASQAGVEKILSALEIPRVRRLSQVVVMKSKNDQGITNKCKMNLQDRRGNDIGFIGFHQFNNIAK
LVASNWYNRQIERRSRRLGCSWEFIPVDDGWGERPL
SEQ ID NO: 83
(Modified BoNT/A1 H_{cc} domain (N1188R, D1213R, G1215R, N1216R,
N1242R, N1243R, S1274R, T1277R, M1144A) Polypeptide Sequence)
YYMLNLYDPNKYVDVNNVGIRGYMYLKGPRGSVATTNIYLNSSLYRGTKFIIKKYASGNKDNIVRNNDRVYINVV
VKRKEYRLATNASQAGVEKILSALEIPRVRRLSQVVVMKSKNDQGITNKCKMNLQDRRGNDIGFIGFHQFNNIAK
LVASNWYNRQIERRSRRLGCSWEFIPVDDGWGERPL
SEQ ID NO: 84
(Modified BoNT/A1 H_{cc} domain (N1188R, D1213R, G1215R, N1216R,
N1242R, N1243R, S1274R, T1277R, M11441) Polypeptide Sequence)
YYMLNLYDPNKYVDVNNVGIRGYMYLKGPRGSVITTNIYLNSSLYRGTKFIIKKYASGNKDNIVRNNDRVYINVV
VKRKEYRLATNASQAGVEKILSALEIPRVRRLSQVVVMKSKNDQGITNKCKMNLQDRRGNDIGFIGFHQFNNIAK
LVASNWYNRQIERRSRRLGCSWEFIPVDDGWGERPL
SEQ ID NO: 85
(Modified BoNT/A1 H_{cc} domain (N1188R, D1213R, G1215R, N1216R,
N1242R, N1243R, S1274R, T1277R, M1144 Deletion) Polypeptide Sequence)
YYMLNLYDPNKYVDVNNVGIRGYMYLKGPRGSVTTNIYLNSSLYRGTKFIIKKYASGNKDNIVRNNDRVYINVVV
KRKEYRLATNASQAGVEKILSALEIPRVRRLSQVVVMKSKNDQGITNKCKMNLQDRRGNDIGFIGFHQFNNIAKL
VASNWYNRQIERRSRRLGCSWEFIPVDDGWGERPL
SEQ ID NO: 86
(BoNT/A3 H_{cc} domain Polypeptide Sequence)
YYMLNLFDPNKYVDVNNIGIRGYMYLKGPRGSVMTTNIYLNSTLYMGTKFIIKKYASGNEDNIVRNNDRVYINVV
VKNKEYRLATNASQAGVEKILSALEIPDVGNLSQVVVMKSKDDQGIRNKCKMNLQDNNGNDIGFVGFHLYDNIAK
LVASNWYNRQVGKASRTFGCSWEFIPVDDGWGESSL
SEQ ID NO: 87
(Modified BoNT/A3 H_{cc} domain (M1144V) Polypeptide Sequence)
YYMLNLFDPNKYVDVNNIGIRGYMYLKGPRGSVVTTNIYLNSTLYMGTKFIIKKYASGNEDNIVRNNDRVYINVV
VKNKEYRLATNASQAGVEKILSALEIPDVGNLSQVVVMKSKDDQGIRNKCKMNLQDNNGNDIGFVGFHLYDNIAK
LVASNWYNRQVGKASRTFGCSWEFIPVDDGWGESSL
SEQ ID NO: 88
(Modified BoNT/A3 H_{cc} domain (M1144G) Polypeptide Sequence)
YYMLNLFDPNKYVDVNNIGIRGYMYLKGPRGSVGTTNIYLNSTLYMGTKFIIKKYASGNEDNIVRNNDRVYINVV
VKNKEYRLATNASQAGVEKILSALEIPDVGNLSQVVVMKSKDDQGIRNKCKMNLQDNNGNDIGFVGFHLYDNIAK
LVASNWYNRQVGKASRTFGCSWEFIPVDDGWGESSL
SEQ ID NO: 89
(Modified BoNT/A3 H_{cc} domain (M1144L) Polypeptide Sequence)
YYMLNLFDPNKYVDVNNIGIRGYMYLKGPRGSVLTTNIYLNSTLYMGTKFIIKKYASGNEDNIVRNNDRVYINVV
VKNKEYRLATNASQAGVEKILSALEIPDVGNLSQVVVMKSKDDQGIRNKCKMNLQDNNGNDIGFVGFHLYDNIAK
LVASNWYNRQVGKASRTFGCSWEFIPVDDGWGESSL
SEQ ID NO: 90
(Modified BoNT/A3 H_{cc} domain (M1144T) Polypeptide Sequence)
YYMLNLFDPNKYVDVNNIGIRGYMYLKGPRGSVTTTNIYLNSTLYMGTKFIIKKYASGNEDNIVRNNDRVYINVV
VKNKEYRLATNASQAGVEKILSALEIPDVGNLSQVVVMKSKDDQGIRNKCKMNLQDNNGNDIGFVGFHLYDNIAK
LVASNWYNRQVGKASRTFGCSWEFIPVDDGWGESSL
SEQ ID NO: 91
(Modified BoNT/A3 H_{cc} domain (M1144A) Polypeptide Sequence)
YYMLNLFDPNKYVDVNNIGIRGYMYLKGPRGSVATTNIYLNSTLYMGTKFIIKKYASGNEDNIVRNNDRVYINVV
VKNKEYRLATNASQAGVEKILSALEIPDVGNLSQVVVMKSKDDQGIRNKCKMNLQDNNGNDIGFVGFHLYDNIAK
LVASNWYNRQVGKASRTFGCSWEFIPVDDGWGESSL
SEQ ID NO: 92
(Modified BoNT/A3 H_{cc} domain (M11441) Polypeptide Sequence)
YYMLNLFDPNKYVDVNNIGIRGYMYLKGPRGSVITTNIYLNSTLYMGTKFIIKKYASGNEDNIVRNNDRVYINVV
VKNKEYRLATNASQAGVEKILSALEIPDVGNLSQVVVMKSKDDQGIRNKCKMNLQDNNGNDIGFVGFHLYDNIAK
LVASNWYNRQVGKASRTFGCSWEFIPVDDGWGESSL
SEQ ID NO: 93
(Modified BoNT/A3 H_{cc} domain (M1144 Deletion) Polypeptide Sequence)
YYMLNLFDPNKYVDVNNIGIRGYMYLKGPRGSVTTNIYLNSTLYMGTKFIIKKYASGNEDNIVRNNDRVYINVVV
KNKEYRLATNASQAGVEKILSALEIPDVGNLSQVVVMKSKDDQGIRNKCKMNLQDNNGNDIGFVGFHLYDNIAKL
VASNWYNRQVGKASRTFGCSWEFIPVDDGWGESSL
SEQ ID NO: 94
(BoNT/A4 H_{cc} domain Polypeptide Sequence)
YYMLNLYDPNKYVDVNNVGIRGYMYLKGPRDNVMTTNIYLNSSLYMGTKFIIKKYASGNKDNIVRNNDRVYINVV
VKNKEYRLATNASQAGVEKILSALEIPDVGNLSQVVVMKSKNDQGITNKCKMNLQDNNGNDIGFIGFHQFNNIAK
LVASNWYNRQIERSSRTLGCSWEFIPVDDGWRERPL
SEQ ID NO: 95
(Modified BoNT/A4 H_{cc} domain (M1144V) Polypeptide Sequence)
YYMLNLYDPNKYVDVNNVGIRGYMYLKGPRDNVVTTNIYLNSSLYMGTKFIIKKYASGNKDNIVRNNDRVYINVV
VKNKEYRLATNASQAGVEKILSALEIPDVGNLSQVVVMKSKNDQGITNKCKMNLQDNNGNDIGFIGFHQFNNIAK
LVASNWYNRQIERSSRTLGCSWEFIPVDDGWRERPL
SEQ ID NO: 96
(Modified BoNT/A4 H_{cc} domain (M1144G) Polypeptide Sequence)
YYMLNLYDPNKYVDVNNVGIRGYMYLKGPRDNVGTTNIYLNSSLYMGTKFIIKKYASGNKDNIVRNNDRVYINVV
VKNKEYRLATNASQAGVEKILSALEIPDVGNLSQVVVMKSKNDQGITNKCKMNLQDNNGNDIGFIGFHQFNNIAK
LVASNWYNRQIERSSRTLGCSWEFIPVDDGWRERPL
SEQ ID NO: 97
(Modified BoNT/A4 H_{cc} domain (M1144L) Polypeptide Sequence)
YYMLNLYDPNKYVDVNNVGIRGYMYLKGPRDNVLTTNIYLNSSLYMGTKFIIKKYASGNKDNIVRNNDRVYINVV
VKNKEYRLATNASQAGVEKILSALEIPDVGNLSQVVVMKSKNDQGITNKCKMNLQDNNGNDIGFIGFHQFNNIAK
LVASNWYNRQIERSSRTLGCSWEFIPVDDGWRERPL
SEQ ID NO: 98
(Modified BoNT/A4 H_{cc} domain (M1144T) Polypeptide Sequence)
YYMLNLYDPNKYVDVNNVGIRGYMYLKGPRDNVTTTNIYLNSSLYMGTKFIIKKYASGNKDNIVRNNDRVYINVV
VKNKEYRLATNASQAGVEKILSALEIPDVGNLSQVVVMKSKNDQGITNKCKMNLQDNNGNDIGFIGFHQFNNIAK
LVASNWYNRQIERSSRTLGCSWEFIPVDDGWRERPL
SEQ ID NO: 99
(Modified BoNT/A4 H_{cc} domain (M1144A) Polypeptide Sequence)
YYMLNLYDPNKYVDVNNVGIRGYMYLKGPRDNVATTNIYLNSSLYMGTKFIIKKYASGNKDNIVRNNDRVYINVV
VKNKEYRLATNASQAGVEKILSALEIPDVGNLSQVVVMKSKNDQGITNKCKMNLQDNNGNDIGFIGFHQFNNIAK
LVASNWYNRQIERSSRTLGCSWEFIPVDDGWRERPL
SEQ ID NO: 100
(Modified BoNT/A4 H_{cc} domain (M11441) Polypeptide Sequence)
YYMLNLYDPNKYVDVNNVGIRGYMYLKGPRDNVITTNIYLNSSLYMGTKFIIKKYASGNKDNIVRNNDRVYINVV
VKNKEYRLATNASQAGVEKILSALEIPDVGNLSQVVVMKSKNDQGITNKCKMNLQDNNGNDIGFIGFHQFNNIAK
LVASNWYNRQIERSSRTLGCSWEFIPVDDGWRERPL
SEQ ID NO: 101
(Modified BoNT/A4 H_{cc} domain (M1144 Deletion) Polypeptide Sequence)
YYMLNLYDPNKYVDVNNVGIRGYMYLKGPRDNVTTNIYLNSSLYMGTKFIIKKYASGNKDNIVRNNDRVYINVVV
KNKEYRLATNASQAGVEKILSALEIPDVGNLSQVVVMKSKNDQGITNKCKMNLQDNNGNDIGFIGFHQENNIAK
LVASNWYNRQIERSSRTLGCSWEFIPVDDGWRERPL
SEQ ID NO: 102
(Modified SV2 Binding Domain Consensus Sequence 1)
RX1X2VX3TTNIYLNSX4LYX5GT, wherein: X1 is D or G; X2 is S or N; X3 is an
amino acid that is resistant to oxidation; X4 is S or T; and X5 is M
or R.
SEQ ID NO: 103
(BoNT/A1 Hall Str (Reference) SV2 Binding Domain)
RGSVMTTNIYLNSSLYRGT
SEQ ID NO: 104
(BoNT/A1 CDC297 SV2 Binding Domain)
RGNVMTTNIYLNSSLYMGT
SEQ ID NO: 105
(BoNT/A3 Loch Maree SV2 Binding Domain)
RGSVMTTNIYLNSTLYMGT
SEQ ID NO: 106
(BoNT/A4 SV2 Binding Domain)
RDNVMTTNIYLNSSLYMGT
SEQ ID NO: 107
(Modified SV2 Binding Domain Consensus Sequence 2)
RGSVXTTNIYLNSSLYRGT, wherein X is an amino acid that is resistant to
oxidation.
SEQ ID NO: 108
(Modified SV2 Binding Domain Consensus Sequence 3)
RGNVXTTNIYLNSSLYMGT, wherein X is an amino acid that is resistant to
oxidation.
SEQ ID NO: 109
(Modified SV2 Binding Domain Consensus Sequence 4)
RGSVXTTNIYLNSTLYMGT, wherein X is an amino acid that is resistant to
oxidation.
SEQ ID NO: 110
(Modified SV2 Binding Domain Consensus Sequence 5)
RDNVXTTNIYLNSSLYMGT, wherein X is an amino acid that is resistant to
oxidation.
SEQ ID NO: 111
(Modified SV2 Binding Domain Consensus Sequence 6)
RX1X2VTTNIYLNSX3LYX4GT, wherein: X1 is D or G; X2 is S or N; X3 is S or
T; and X4 is M or R.
SEQ ID NO: 112
(Modified SV2 Binding Domain A)
RGSVTTNIYLNSSLYRGT
SEQ ID NO: 113
(Modified SV2 Binding Domain B)
RGNVTTNIYLNSSLYMGT
SEQ ID NO: 114
(Modified SV2 Binding Domain C)
RGSVTTNIYLNSTLYMGT
SEQ ID NO: 115
Modified SV2 Binding Domain D)
RDNVTTNIYLNSSLYMGT
SEQ ID NO: 116
(Influenza Virus Haemagglutinin)
GLFGAIAGFIENGWEGMIDGWYG
SEQ ID NO: 117
(Leucine-Based Motif 1)
xDxxxLL, wherein x is any amino acid.
SEQ ID NO: 118
(Leucine-Based Motif 2)
xExxxLL, wherein x is any amino acid.
SEQ ID NO: 119
(Leucine-Based Motif 3)
xExxxIL, wherein x is any amino acid.
SEQ ID NO: 120
(Leucine-Based Motif 4)
xExxxLM, wherein x is any amino acid.
SEQ ID NO: 121
(Tyrosine-based Motif)
Y-x-x-Hy, wherein Hy is a hydrophobic amino acid and wherein x is any
amino acid.
SEQ ID NO: 122
(TEV Cleavage Site)
ENLYFQG
SEQ ID NO: 123
(Thrombin Cleavage Site)
LVPRGS
SEQ ID NO: 124
(PreScission Cleavage Site)
LEVLFQGP
SEQ ID NO: 125
(Enterokinase Cleavage Site)
DDDDK
SEQ ID NO: 126
(Factor Xa Cleavage Site 1)
IEGR
SEQ ID NO: 127
(Factor Xa Cleavage Site 2)
IDGR
SEQ ID NO: 128
(Polypeptide Sequence of BoNT/B - UniProt P10844)
MPVTINNFNYNDPIDNNNIIMMEPPFARGTGRYYKAFKITDRIWIIPERYTFGYKPEDFN
KSSGIFNRDVCEYYDPDYLNTNDKKNIFLQTMIKLFNRIKSKPLGEKLLEMIINGIPYLG
DRRVPLEEFNTNIASVTVNKLISNPGEVERKKGIFANLIIFGPGPVLNENETIDIGIQNH
FASREGFGGIMQMKFCPEYVSVFNNVQENKGASIFNRRGYFSDPALILMHELIHVLHGLY
GIKVDDLPIVPNEKKFFMQSTDAIQAEELYTFGGQDPSIITPSTDKSIYDKVLQNFRGIV
DRLNKVLVCISDPNININIYKNKFKDKYKFVEDSEGKYSIDVESFDKLYKSLMFGFTETN
IAENYKIKTRASYFSDSLPPVKIKNLLDNEIYTIEEGFNISDKDMEKEYRGQNKAINKQA
YEEISKEHLAVYKIQMCKSVKAPGICIDVDNEDLFFIADKNSFSDDLSKNERIEYNTQSN
YIENDFPINELILDTDLISKIELPSENTESLTDFNVDVPVYEKQPAIKKIFTDENTIFQY
LYSQTFPLDIRDISLTSSFDDALLFSNKVYSFFSMDYIKTANKVVEAGLFAGWVKQIVND
FVIEANKSNTMDKIADISLIVPYIGLALNVGNETAKGNFENAFEIAGASILLEFIPELLI
PVVGAFLLESYIDNKNKIIKTIDNALTKRNEKWSDMYGLIVAQWLSTVNTQFYTIKEGMY
KALNYQAQALEEIIKYRYNIYSEKEKSNINIDFNDINSKLNEGINQAIDNINNFINGCSV
SYLMKKMIPLAVEKLLDFDNTLKKNLLNYIDENKLYLIGSAEYEKSKVNKYLKTIMPFDL
SIYTNDTILIEMFNKYNSEILNNIILNLRYKDNNLIDLSGYGAKVEVYDGVELNDKNQFK
LTSSANSKIRVTQNQNIIFNSVFLDFSVSFWIRIPKYKNDGIQNYIHNEYTIINCMKNNS
GWKISIRGNRIIWTLIDINGKTKSVFFEYNIREDISEYINRWFFVTITNNLNNAKIYING
KLESNTDIKDIREVIANGEIIFKLDGDIDRTQFIWMKYFSIFNTELSQSNIEERYKIQSY
SEYLKDFWGNPLMYNKEYYMFNAGNKNSYIKLKKDSPVGEILTRSKYNQNSKYINYRDLY
IGEKFIIRRKSNSQSINDDIVRKEDYIYLDFFNLNQEWRVYTYKYFKKEEEKLFLAPISD
SDEFYNTIQIKEYDEQPTYSCQLLFKKDEESTDEIGLIGIHRFYESGIVFEEYKDYFCIS
KWYLKEVKRKPYNLKLGCNWQFIPKDEGWTE
SEQ ID NO: 129
(Polypeptide Sequence of BoNT/C - UniProt P18640)
MPITINNFNYSDPVDNKNILYLDTHLNTLANEPEKAFRITGNIWVIPDRFSRNSNPNLNK
PPRVTSPKSGYYDPNYLSTDSDKDPFLKEIIKLFKRINSREIGEELIYRLSTDIPFPGNN
NTPINTFDFDVDFNSVDVKTRQGNNWVKTGSINPSVIITGPRENIIDPETSTFKLTNNTF
AAQEGFGALSIISISPRFMLTYSNATNDVGEGRFSKSEFCMDPILILMHELNHAMHNLYG
IAIPNDQTISSVTSNIFYSQYNVKLEYAEIYAFGGPTIDLIPKSARKYFEEKALDYYRSI
AKRLNSITTANPSSFNKYIGEYKQKLIRKYRFVVESSGEVTVNRNKFVELYNELTQIFTE
FNYAKIYNVQNRKIYLSNVYTPVTANILDDNVYDIQNGFNIPKSNLNVLFMGQNLSRNPA
LRKVNPENMLYLFTKFCHKAIDGRSLYNKTLDCRELLVKNTDLPFIGDISDVKTDIFLRK
DINEETEVIYYPDNVSVDQVILSKNTSEHGQLDLLYPSIDSESEILPGENQVFYDNRTQN
VDYLNSYYYLESQKLSDNVEDFTFTRSIEEALDNSAKVYTYFPTLANKVNAGVQGGLFLM
WANDVVEDFTTNILRKDTLDKISDVSAIIPYIGPALNISNSVRRGNFTEAFAVTGVTILL
EAFPEFTIPALGAFVIYSKVQERNEIIKTIDNCLEQRIKRWKDSYEWMMGTWLSRIITQF
NNISYQMYDSLNYQAGAIKAKIDLEYKKYSGSDKENIKSQVENLKNSLDVKISEAMNNIN
KFIRECSVTYLFKNMLPKVIDELNEFDRNTKAKLINLIDSHNIILVGEVDKLKAKVNNSF
QNTIPFNIFSYTNNSLLKDIINEYFNNINDSKILSLQNRKNTLVDTSGYNAEVSEEGDVQ
LNPIFPFDFKLGSSGEDRGKVIVTQNENIVYNSMYESFSISFWIRINKWVSNLPGYTIID
SVKNNSGWSIGIISNFLVFTLKQNEDSEQSINFSYDISNNAPGYNKWFFVTVTNNMMGNM
KIYINGKLIDTIKVKELTGINFSKTITFEINKIPDTGLITSDSDNINMWIRDFYIFAKEL
DGKDINILFNSLQYTNVVKDYWGNDLRYNKEYYMVNIDYLNRYMYANSRQIVFNTRRNNN
DFNEGYKIIIKRIRGNTNDTRVRGGDILYFDMTINNKAYNLFMKNETMYADNHSTEDIYA
IGLREQTKDINDNIIFQIQPMNNTYYYASQIFKSNFNGENISGICSIGTYRFRLGGDWYR
HNYLVPTVKQGNYASLLESTSTHWGFVPVSE
SEQ ID NO: 130
(Polypeptide Sequence of BoNT/D - UniProt P19321)
MTWPVKDFNYSDPVNDNDILYLRIPQNKLITTPVKAFMITQNIWVIPERFSSDTNPSLSK
PPRPTSKYQSYYDPSYLSTDEQKDTFLKGIIKLFKRINERDIGKKLINYLVVGSPFMGDS
STPEDTFDFTRHTTNIAVEKFENGSWKVTNIITPSVLIFGPLPNILDYTASLTLQGQQSN
PSFEGFGTLSILKVAPEFLLTFSDVTSNQSSAVLGKSIFCMDPVIALMHELTHSLHQLYG
INIPSDKRIRPQVSEGFFSQDGPNVQFEELYTFGGLDVEIIPQIERSQLREKALGHYKDI
AKRLNNINKTIPSSWISNIDKYKKIFSEKYNFDKDNTGNFVVNIDKFNSLYSDLTNVMSE
VVYSSQYNVKNRTHYFSRHYLPVFANILDDNIYTIRDGFNLINKGFNIENSGQNIERNPA
LQKLSSESVVDLFTKVCLRLTKNSRDDSTCIKVKNNRLPYVADKDSISQEIFENKIITDE
TNVQNYSDKFSLDESILDGQVPINPEIVDPLLPNVNMEPLNLPGEEIVFYDDITKYVDYL
NSYYYLESQKLSNNVENITLTTSVEEALGYSNKIYTFLPSLAEKVNKGVQAGLFLNWANE
VVEDFTTNIMKKDTLDKISDVSVIIPYIGPALNIGNSALRGNFNQAFATAGVAFLLEGFP
EFTIPALGVFTFYSSIQEREKIIKTIENCLEQRVKRWKDSYQWMVSNWLSRITTQFNHIN
YQMYDSLSYQADAIKAKIDLEYKKYSGSDKENIKSQVENLKNSLDVKISEAMNNINKFIR
ECSVTYLFKNMLPKVIDELNKFDLRTKTELINLIDSHNIILVGEVDRLKAKVNESFENTM
PFNIFSYTNNSLLKDIINEYFNSINDSKILSLQNKKNALVDTSGYNAEVRVGDNVQLNTI
YTNDFKLSSSGDKIIVNLNNNILYSAIYENSSVSFWIKISKDLTNSHNEYTIINSIEQNS
GWKLCIRNGNIEWILQDVNRKYKSLIFDYSESLSHTGYTNKWFFVTITNNIMGYMKLYIN
GELKQSQKIEDLDEVKLDKTIVFGIDENIDENQMLWIRDFNIFSKELSNEDINIVYEGQI
LRNVIKDYWGNPLKFDTEYYIINDNYIDRYIAPESNVLVLVQYPDRSKLYTGNPITIKSV
SDKNPYSRILNGDNIILHMLYNSRKYMIIRDTDTIYATQGGECSQNCVYALKLQSNLGNY
GIGIFSIKNIVSKNKYCSQIFSSFRENTMLLADIYKPWRFSFKNAYTPVAVTNYETKLLS
TSSFWKFISRDPGWVE
SEQ ID NO: 131
(Polypeptide Sequence of BoNT/E - UniProt Q00496)
MPKINSFNYNDPVNDRTILYIKPGGCQEFYKSFNIMKNIWIIPERNVIGTTPQDFHPPTS
LKNGDSSYYDPNYLQSDEEKDRFLKIVTKIFNRINNNLSGGILLEELSKANPYLGNDNTP
DNQFHIGDASAVEIKFSNGSQDILLPNVIIMGAEPDLFETNSSNISLRNNYMPSNHRFGS
IAIVTFSPEYSFRFNDNCMNEFIQDPALTLMHELIHSLHGLYGAKGITTKYTITQKQNPL
ITNIRGTNIEEFLTFGGTDLNIITSAQSNDIYTNLLADYKKIASKLSKVQVSNPLLNPYK
DVFEAKYGLDKDASGIYSVNINKFNDIFKKLYSFTEFDLRTKFQVKCRQTYIGQYKYFKL
SNLLNDSIYNISEGYNINNLKVNFRGQNANLNPRIITPITGRGLVKKIIRFCKNIVSVKG
IRKSICIEINNGELFFVASENSYNDDNINTPKEIDDTVTSNNNYENDLDQVILNFNSESA
PGLSDEKLNLTIQNDAYIPKYDSNGTSDIEQHDVNELNVFFYLDAQKVPEGENNVNLTSS
IDTALLEQPKIYTFFSSEFINNVNKPVQAALFVSWIQQVLVDFTTEANQKSTVDKIADIS
IVVPYIGLALNIGNEAQKGNFKDALELLGAGILLEFEPELLIPTILVFTIKSFLGSSDNK
NKVIKAINNALKERDEKWKEVYSFIVSNWMTKINTQFNKRKEQMYQALQNQVNAIKTIIE
SKYNSYTLEEKNELTNKYDIKQIENELNQKVSIAMNNIDRFLTESSISYLMKIINEVKIN
KLREYDENVKTYLLNYIIQHGSILGESQQELNSMVTDTLNNSIPFKLSSYTDDKILISYF
NKFFKRIKSSSVLNMRYKNDKYVDTSGYDSNININGDVYKYPTNKNQFGIYNDKLSEVNI
SQNDYIIYDNKYKNFSISFWVRIPNYDNKIVNVNNEYTIINCMRDNNSGWKVSLNHNEII
WTFEDNRGINQKLAFNYGNANGISDYINKWIFVTITNDRLGDSKLYINGNLIDQKSILNL
GNIHVSDNILFKIVNCSYTRYIGIRYFNIFDKELDETEIQTLYSNEPNTNILKDFWGNYL
LYDKEYYLLNVLKPNNFIDRRKDSTLSINNIRSTILLANRLYSGIKVKIQRVNNSSTNDN
LVRKNDQVYINFVASKTHLFPLYADTATTNKEKTIKISSSGNRFNQVVVMNSVGNCTMNF
KNNNGNNIGLLGFKADTVVASTWYYTHMRDHTNSNGCFWNFISEEHGWQEK
SEQ ID NO: 132
(Polypeptide Sequence of BoNT/F - UniProt A7GBG3)
MPVVINSFNYNDPVNDDTILYMQIPYEEKSKKYYKAFEIMRNVWIIPERNTIGTDPSDFD
PPASLENGSSAYYDPNYLTTDAEKDRYLKTTIKLFKRINSNPAGEVLLQEISYAKPYLGN
EHTPINEFHPVTRTTSVNIKSSTNVKSSIILNLLVLGAGPDIFENSSYPVRKLMDSGGVY
DPSNDGFGSINIVTFSPEYEYTFNDISGGYNSSTESFIADPAISLAHELIHALHGLYGAR
GVTYKETIKVKQAPLMIAEKPIRLEEFLTFGGQDLNIITSAMKEKIYNNLLANYEKIATR
LSRVNSAPPEYDINEYKDYFQWKYGLDKNADGSYTVNENKFNEIYKKLYSFTEIDLANKF
KVKCRNTYFIKYGFLKVPNLLDDDIYTVSEGFNIGNLAVNNRGQNIKLNPKIIDSIPDKG
LVEKIVKFCKSVIPRKGTKAPPRLCIRVNNRELFFVASESSYNENDINTPKEIDDTTNLN
NNYRNNLDEVILDYNSETIPQISNQTLNTLVQDDSYVPRYDSNGTSEIEEHNVVDLNVFF
YLHAQKVPEGETNISLTSSIDTALSEESQVYTFFSSEFINTINKPVHAALFISWINQVIR
DFTTEATQKSTFDKIADISLVVPYVGLALNIGNEVQKENFKEAFELLGAGILLEFVPELL
IPTILVFTIKSFIGSSENKNKIIKAINNSLMERETKWKEIYSWIVSNWLTRINTQFNKRK
EQMYQALQNQVDAIKTVIEYKYNNYTSDERNRLESEYNINNIREELNKKVSLAMENIERF
ITESSIFYLMKLINEAKVSKLREYDEGVKEYLLDYISEHRSILGNSVQELNDLVTSTLNN
SIPFELSSYTNDKILILYFNKLYKKIKDNSILDMRYENNKFIDISGYGSNISINGDVYIY
STNRNQFGIYSSKPSEVNIAQNNDIIYNGRYQNFSISFWVRIPKYFNKVNLNNEYTIIDC
IRNNNSGWKISLNYNKIIWTLQDTAGNNQKLVFNYTQMISISDYINKWIFVTITNNRLGN
SRIYINGNLIDEKSISNLGDIHVSDNILFKIVGCNDTRYVGIRYFKVFDTELGKTEIETL
YSDEPDPSILKDFWGNYLLYNKRYYLLNLLRTDKSITQNSNFLNINQQRGVYQKPNIFSN
TRLYTGVEVIIRKNGSTDISNTDNFVRKNDLAYINVVDRDVEYRLYADISIAKPEKIIKL
IRTSNSNNSLGQIIVMDSIGNNCTMNFQNNNGGNIGLLGFHSNNLVASSWYYNNIRKNTS
SNGCFWSFISKEHGWQEN
SEQ ID NO: 133
(Polypeptide Sequence of BoNT/G - UniProt Q60393)
MPVNIKXFNYNDPINNDDIIMMEPFNDPGPGTYYKAFRIIDRIWIVPERFTYGFQPDQFN
ASTGVFSKDVYEYYDPTYLKTDAEKDKFLKTMIKLFNRINSKPSGQRLLDMIVDAIPYLG
NASTPPDKFAANVANVSINKKIIQPGAEDQIKGLMTNLIIFGPGPVLSDNFTDSMIMNGH
SPISEGFGARMMIRFCPSCLNVFNNVQENKDTSIFSRRAYFADPALTLMHELIHVLHGLY
GIKISNLPITPNTKEFFMQHSDPVQAEELYTFGGHDPSVISPSTDMNIYNKALQNFQDIA
NRLNIVSSAQGSGIDISLYKQIYKNKYDFVEDPNGKYSVDKDKFDKLYKALMFGFTETNL
AGEYGIKTRYSYFSEYLPPIKTEKLLDNTIYTQNEGFNIASKNLKTEFNGQNKAVNKEAY
EEISLEHLVIYRIAMCKPVMYKNTGKSEQCIIVNNEDLFFIANKDSFSKDLAKAETIAYN
TQNNTIENNFSIDQLILDNDLSSGIDLPNENTEPFTNFDDIDIPVYIKQSALKKIFVDGD
SLFEYLHAQTFPSNIENLQLTNSLNDALRNNNKVYTFFSTNLVEKANTVVGASLFVNWVK
GVIDDFTSESTQKSTIDKVSDVSIIIPYIGPALNVGNETAKENFKNAFEIGGAAILMEFI
PELIVPIVGFFTLESYVGNKGHIIMTISNALKKRDQKWTDMYGLIVSQWLSTVNTQFYTI
KERMYNALNNQSQAIEKIIEDQYNRYSEEDKMNINIDFNDIDFKLNQSINLAINNIDDFI
NQCSISYLMNRMIPLAVKKLKDFDDNLKRDLLEYIDTNELYLLDEVNILKSKVNRHLKDS
IPFDLSLYTKDTILIQVFNNYISNISSNAILSLSYRGGRLIDSSGYGATMNVGSDVIFND
IGNGQFKLNNSENSNITAHQSKFVVYDSMFDNFSINFWVRTPKYNNNDIQTYLQNEYTII
SCIKNDSGWKVSIKGNRIIWTLIDVNAKSKSIFFEYSIKDNISDYINKWFSITITNDRLG
NANIYINGSLKKSEKILNLDRINSSNDIDFKLINCTDTTKFVWIKDFNIFGRELNATEVS
SLYWIQSSTNTLKDFWGNPLRYDTQYYLFNQGMQNIYIKYFSKASMGETAPRTNFNNAAI
NYQNLYLGLRFIIKKASNSRNINNDNIVREGDYIYLNIDNISDESYRVYVLVNSKEIQTQ
LFLAPINDDPTFYDVLQIKKYYEKTTYNCQILCEKDTKTFGLFGIGKFVKDYGYVWDTYD
NYFCISQWYLRRISENINKLRLGCNWQFIPVDEGWTE
SEQ ID NO: 134
(Polypeptide Sequence of TeNT - UniProt P04958)
MPITINNFRYSDPVNNDTIIMMEPPYCKGLDIYYKAFKITDRIWIVPERYEFGTKPEDFN
PPSSLIEGASEYYDPNYLRTDSDKDRFLQTMVKLFNRIKNNVAGEALLDKIINAIPYLGN
SYSLLDKFDTNSNSVSFNLLEQDPSGATTKSAMLTNLIIFGPGPVLNKNEVRGIVLRVDN
KNYFPCRDGFGSIMQMAFCPEYVPTFDNVIENITSLTIGKSKYFQDPALLLMHELIHVLH
GLYGMQVSSHEIIPSKQEIYMQHTYPISAEELFTFGGQDANLISIDIKNDLYEKTLNDYK
AIANKLSQVTSCNDPNIDIDSYKQIYQQKYQFDKDSNGQYIVNEDKFQILYNSIMYGFTE
IELGKKFNIKTRLSYFSMNHDPVKIPNLLDDTIYNDTEGFNIESKDLKSEYKGQNMRVNT
NAFRNVDGSGLVSKLIGLCKKIIPPTNIRENLYNRTASLTDLGGELCIKIKNFDLTFIAE
KNSFSEEPFQDEIVSYNTKNKPLNFNYSLDKIIVDYNLQSKITLPNDRTTPVTKGIPYAP
EYKSNAASTIEIHNIDDNTIYQYLYAQKSPTTLQRITMTNSVDDALINSTKIYSYFPSVI
SKVNQGAQGILFLQWVRDIIDDFTNESSQKTTIDKISDVSTIVPYIGPALNIVKQGYEGN
FIGALETTGVVLLLEYIPEITLPVIAALSIAESSTQKEKIIKTIDNFLEKRYEKWIEVYK
LVKAKWLGTVNTQFQKRSYQMYRSLEYQVDAIKKIIDYEYKIYSGPDKEQIADEINNLKN
KLEEKANKAMININIFMRESSRSFLVNQMINEAKKQLLEFDTQSKNILMQYIKANSKFIG
ITELKKLESKINKVFSTPIPFSYSKNLDCWVDNEEDIDVILKKSTILNLDINNDIISDIS
GFNSSVITYPDAQLVPGINGKAIHLVNNESSEVIVHKAMDIEYNDMFNNFTVSFWLRVPK
VSASHLEQYGTNEYSIISSMKKHSLSIGSGWSVSLKGNNLIWTLKDSAGEVRQITFRDLP
DKFNAYLANKWVFITITNDRLSSANLYINGVLMGSAEITGLGAIREDNNITLKLDRCNNN
NQYVSIDKFRIFCKALNPKEIEKLYTSYLSITFLRDFWGNPLRYDTEYYLIPVASSSKDV
QLKNITDYMYLTNAPSYTNGKLNIYYRRLYNGLKFIIKRYTPNNEIDSFVKSGDFIKLYV
SYNNNEHIVGYPKDGNAFNNLDRILRVGYNAPGIPLYKKMEAVKLRDLKTYSVQLKLYDD
KNASLGLVGTHNGQIGNDPNRDILIASNWYFNHLKDKILGCDWYFVPTDEGWTND
SEQ ID NO: 135
(Polypeptide Sequence of BoNT/X)
MKLEINKFNYNDPIDGINVITMRPPRHSDKINKGKGPFKAFQVIKNIWIVPERYNFTNNT
NDLNIPSEPIMEADAIYNPNYLNTPSEKDEFLQGVIKVLERIKSKPEGEKLLELISSSIP
LPLVSNGALTLSDNETIAYQENNNIVSNLQANLVIYGPGPDIANNATYGLYSTPISNGEG
TLSEVSFSPFYLKPFDESYGNYRSLVNIVNKFVKREFAPDPASTLMHELVHVTHNLYGIS
NRNFYYNFDTGKIETSRQQNSLIFEELLTFGGIDSKAISSLIIKKIIETAKNNYTTLISE
RLNTVTVENDLLKYIKNKIPVQGRLGNFKLDTAEFEKKLNTILFVLNESNLAQRFSILVR
KHYLKERPIDPIYVNILDDNSYSTLEGFNISSQGSNDFQGQLLESSYFEKIESNALRAFI
KICPRNGLLYNAIYRNSKNYLNNIDLEDKKTTSKTNVSYPCSLLNGCIEVENKDLFLISN
KDSLNDINLSEEKIKPETTVFFKDKLPPQDITLSNYDFTEANSIPSISQQNILERNEELY
EPIRNSLFEIKTIYVDKLTTFHFLEAQNIDESIDSSKIRVELTDSVDEALSNPNKVYSPF
KNMSNTINSIETGITSTYIFYQWLRSIVKDFSDETGKIDVIDKSSDTLAIVPYIGPLLNI
GNDIRHGDFVGAIELAGITALLEYVPEFTIPILVGLEVIGGELAREQVEAIVNNALDKRD
QKWAEVYNITKAQWWGTIHLQINTRLAHTYKALSRQANAIKMNMEFQLANYKGNIDDKAK
IKNAISETEILLNKSVEQAMKNTEKFMIKLSNSYLTKEMIPKVQDNLKNFDLETKKTLDK
FIKEKEDILGTNLSSSLRRKVSIRLNKNIAFDINDIPFSEFDDLINQYKNEIEDYEVLNL
GAEDGKIKDLSGTTSDINIGSDIELADGRENKAIKIKGSENSTIKIAMNKYLRFSATDNF
SISFWIKHPKPTNLLNNGIEYTLVENFNQRGWKISIQDSKLIWYLRDHNNSIKIVTPDYI
AFNGWNLITITNNRSKGSIVYVNGSKIEEKDISSIWNTEVDDPIIFRLKNNRDTQAFTLL
DQFSIYRKELNQNEVVKLYNYYFNSNYIRDIWGNPLQYNKKYYLQTQDKPGKGLIREYWS
SFGYDYVILSDSKTITFPNNIRYGALYNGSKVLIKNSKKLDGLVRNKDFIQLEIDGYNMG
ISADRFNEDTNYIGTTYGTTHDLTTDFEIIQRQEKYRNYCQLKTPYNIFHKSGLMSTETS
KPTFHDYRDWVYSSAWYFQNYENLNLRKHTKINWYFIPKDEGWDED
SEQ ID NO: 136
(Modified BoNT/A1 (M1144V) Nucleic Acid Sequence)
ATGCCGTTTGTGAACAAACAGTTCAACTATAAAGATCCGGTGAACGGTGTTGATATCGCCTATATCAA
AATTCCGAATGCAGGTCAGATGCAGCCGGTTAAAGCCTTTAAAATCCATAACAAAATTTGGGTGATTC
CGGAACGTGATACCTTTACCAATCCGGAAGAAGGTGATCTGAATCCGCCTCCGGAAGCAAAACAGGTT
CCGGTTAGCTATTATGATAGCACCTATCTGAGCACCGATAACGAGAAAGATAACTATCTGAAAGGTGT
GACCAAACTGTTTGAACGCATTTATAGTACCGATCTGGGTCGTATGCTGCTGACCAGCATTGTTCGTG
GTATTCCGTTTTGGGGTGGTAGCACCATTGATACCGAACTGAAAGTTATTGACACCAACTGCATTAAT
GTGATTCAGCCGGATGGTAGCTATCGTAGCGAAGAACTGAATCTGGTTATTATTGGTCCGAGCGCAGA
TATCATTCAGTTTGAATGTAAATCCTTTGGCCACGAAGTTCTGAATCTGACCCGTAATGGTTATGGTA
GTACCCAGTATATTCGTTTCAGTCCGGATTTTACCTTTGGCTTTGAAGAAAGCCTGGAAGTTGATACA
AATCCGCTGTTAGGTGCAGGTAAATTTGCAACCGATCCGGCAGTTACCCTGGCACATGAACTGATTCA
TGCCGGTCATCGTCTGTATGGTATTGCAATTAATCCGAACCGTGTGTTCAAAGTGAATACCAACGCAT
ATTATGAAATGAGCGGTCTGGAAGTGTCATTTGAAGAACTGCGTACCTTTGGTGGTCATGATGCCAAA
TTTATCGATAGCCTGCAAGAAAATGAATTTCGCCTGTACTACTATAACAAATTCAAGGATATTGCGAG
CACCCTGAATAAAGCCAAAAGCATTGTTGGCACCACCGCAAGCCTGCAGTATATGAAAAATGTGTTTA
AAGAAAAATATCTGCTGAGCGAAGATACCAGCGGTAAATTTAGCGTTGACAAACTGAAATTCGATAAA
CTGTACAAGATGCTGACCGAGATTTATACCGAAGATAACTTCGTGAAGTTTTTCAAAGTGCTGAACCG
CAAAACCTACCTGAACTTTGATAAAGCCGTGTTCAAAATCAACATCGTGCCGAAAGTGAACTATACCA
TCTATGATGGTTTTAACCTGCGCAATACCAATCTGGCAGCAAACTTTAATGGTCAGAACACCGAAATC
AACAACATGAACTTTACCAAACTGAAGAACTTCACCGGTCTGTTCGAATTTTACAAACTGCTGTGTGT
TCGTGGCATTATTACCtCCAAAACCAAAAGCCTGGATAAAGGTTATAACAAAGCCCTGAATGACCTGT
GCATTAAAGTGAATAATTGGGACCTGTTTTTTAGCCCGAGCGAAGATAACTTTACCAACGATCTGAAT
AAAGGCGAAGAAATTACCAGCGATACCAATATTGAAGCAGCCGAAGAAAACATTAGCCTGGATCTGAT
TCAGCAGTATTATCTGACCTTCAACTTTGATAACGAGCCGGAAAATATCAGCATTGAAAATCTGAGCA
GCGATATTATTGGTCAGCTGGAACTGATGCCGAATATTGAACGTTTTCCGAACGGCAAAAAATACGAG
CTGGATAAATACACCATGTTCCATTATCTGCGTGCCCAAGAATTTGAACATGGTAAAAGCCGTATTGC
CCTGACCAATTCAGTTAATGAAGCACTGCTGAACCCGAGCCGTGTTTATACCTTTTTTAGCAGCGATT
ACGTGAAAAAGGTGAACAAAGCAACCGAAGCAGCAATGTTTTTAGGTTGGGTTGAACAGCTGGTGTAT
GATTTCACCGATGAAACCAGCGAAGTTAGCACCACCGATAAAATTGCAGATATCACCATTATCATCCC
GTATATTGGTCCGGCACTGAATATTGGCAATATGCTGTATAAAGATGATTTCGTGGGTGCCCTGATTT
TTAGCGGTGCAGTTATTCTGCTGGAATTTATTCCGGAAATTGCCATTCCGGTTCTGGGCACCTTTGCA
CTGGTTAGCTATATTGCAAATAAAGTTCTGACCGTGCAGACCATTGATAATGCACTGAGCAAACGTAA
CGAGAAATGGGATGAAGTGTACAAATATATCGTGACCAATTGGCTGGCCAAAGTTAATACCCAGATTG
ATCTGATCCGCAAAAAAATGAAAGAAGCCCTGGAAAATCAGGCAGAAGCAACCAAAGCCATTATCAAC
TATCAGTATAACCAGTACACCGAAGAAGAGAAAAACAACATCAACTTCAACATCGATGACCTGAGCAG
CAAACTGAATGAAAGCATCAATAAGGCCATGATTAACATCAACAAATTTCTGAATCAGTGCAGCGTGA
GCTATCTGATGAATAGCATGATTCCGTATGGTGTGAAACGCCTGGAAGATTTTGATGCAAGCCTGAAA
GATGCGCTGCTGAAATATATCTATGATAATCGTGGCACCCTGATTGGCCAGGTTGATCGTCTGAAAGA
TAAAGTTAACAATACCCTGAGTACCGACATTCCGTTTCAGCTGAGCAAATATGTTGATAATCAGCGTC
TGCTGAGCACCTTTACCGAATATATCAAGAACATCATCAACACCAGCATTCTGAATCTGCGCTATGAA
AGCAATCATCTGATCGATCTGAGCCGTTATGCAAGCAAAATCAACATTGGTAGCAAAGTGAACTTCGA
CCCGATTGATAAAAACCAGATTCAGCTGTTTAATCTGGAAAGCAGCAAAATCGAAGTGATCCTGAAAA
ACGCCATTGTGTATAACAGCATGTATGAGAATTTCTCGACCAGCTTTTGGATTCGCATTCCGAAATAC
TTTAATAGCATCAGCCTGAACAACGAGTACACCATTATTAACTGCATGGAAAACAATAGCGGTTGGAA
AGTGAGCCTGAATTATGGTGAAATTATCTGGACCCTGCAGGATACCCAAGAAATCAAACAGCGTGTTG
TGTTCAAATACAGCCAGATGATTAATATCAGCGACTATATCAACCGCTGGATCTTTGTTACCATTACC
AATAATCGCCTGAATAACAGCAAGATCTATATTAACGGTCGCCTGATTGATCAGAAACCGATTAGCAA
TCTGGGCAATATTCATGCGAGCAACAACATTATGTTTAAACTGGATGGTTGCCGTGATACCCATCGTT
ATATTTGGATCAAATACTTCAACCTGTTTGATAAAGAACTGAACGAAAAAGAAATTAAAGACCTGTAC
GACAACCAGAGCAATTCCGGTATTCTGAAAGACTTTTGGGGAGATTATCTGCAGTATGACAAACCGTA
TTATATGCTGAACCTGTATGACCCGAACAAATATGTGGATGTGAACAATGTTGGTATCCGTGGCTATA
TGTATCTGAAAGGTCCGCGTGGTAGCGTTqTGACCACCAACATTTATCTGAATAGCAGCCTGTATCGC
GGTACGAAATTTATCATTAAAAAGTATGCCAGCGGCAACAAGGATAATATTGTGCGTAATAATGATCG
CGTGTACATTAACGTTGTGGTGAAGAATAAAGAATATCGCCTGGCAACCAATGCAAGCCAGGCAGGCG
TTGAAAAAATTCTGAGCGCACTGGAAATTCCGGATGTTGGTAATCTGAGCCAGGTTGTTGTTATGAAA
AGCAAAAATGATCAGGGCATCACCAACAAGTGCAAAATGAATCTGCAGGACAATAACGGCAACGATAT
TGGTTTTATTGGCTTCCACCAGTTCAACAATATTGCGAAACTGGTTGCAAGCAATTGGTATAATCGTC
AGATTGAACGTAGCAGTCGTACCCTGGGTTGTAGCTGGGAATTTATCCCTGTGGATGATGGTTGGGGT
GAACGTCCGCTGTAA
SEQ ID NO: 137
(Modified BoNT/A1 (M1144G) Nucleic Acid Sequence)
ATGCCGTTTGTGAACAAACAGTTCAACTATAAAGATCCGGTGAACGGTGTTGATATCGCCTATATCAA
AATTCCGAATGCAGGTCAGATGCAGCCGGTTAAAGCCTTTAAAATCCATAACAAAATTTGGGTGATTC
CGGAACGTGATACCTTTACCAATCCGGAAGAAGGTGATCTGAATCCGCCTCCGGAAGCAAAACAGGTT
CCGGTTAGCTATTATGATAGCACCTATCTGAGCACCGATAACGAGAAAGATAACTATCTGAAAGGTGT
GACCAAACTGTTTGAACGCATTTATAGTACCGATCTGGGTCGTATGCTGCTGACCAGCATTGTTCGTG
GTATTCCGTTTTGGGGTGGTAGCACCATTGATACCGAACTGAAAGTTATTGACACCAACTGCATTAAT
GTGATTCAGCCGGATGGTAGCTATCGTAGCGAAGAACTGAATCTGGTTATTATTGGTCCGAGCGCAGA
TATCATTCAGTTTGAATGTAAATCCTTTGGCCACGAAGTTCTGAATCTGACCCGTAATGGTTATGGTA
GTACCCAGTATATTCGTTTCAGTCCGGATTTTACCTTTGGCTTTGAAGAAAGCCTGGAAGTTGATACA
AATCCGCTGTTAGGTGCAGGTAAATTTGCAACCGATCCGGCAGTTACCCTGGCACATGAACTGATTCA
TGCCGGTCATCGTCTGTATGGTATTGCAATTAATCCGAACCGTGTGTTCAAAGTGAATACCAACGCAT
ATTATGAAATGAGCGGTCTGGAAGTGTCATTTGAAGAACTGCGTACCTTTGGTGGTCATGATGCCAAA
TTTATCGATAGCCTGCAAGAAAATGAATTTCGCCTGTACTACTATAACAAATTCAAGGATATTGCGAG
CACCCTGAATAAAGCCAAAAGCATTGTTGGCACCACCGCAAGCCTGCAGTATATGAAAAATGTGTTTA
AAGAAAAATATCTGCTGAGCGAAGATACCAGCGGTAAATTTAGCGTTGACAAACTGAAATTCGATAAA
CTGTACAAGATGCTGACCGAGATTTATACCGAAGATAACTTCGTGAAGTTTTTCAAAGTGCTGAACCG
CAAAACCTACCTGAACTTTGATAAAGCCGTGTTCAAAATCAACATCGTGCCGAAAGTGAACTATACCA
TCTATGATGGTTTTAACCTGCGCAATACCAATCTGGCAGCAAACTTTAATGGTCAGAACACCGAAATC
AACAACATGAACTTTACCAAACTGAAGAACTTCACCGGTCTGTTCGAATTTTACAAACTGCTGTGTGT
TCGTGGCATTATTACCtcCAAAACCAAAAGCCTGGATAAAGGTTATAACAAAGCCCTGAATGACCTGT
GCATTAAAGTGAATAATTGGGACCTGTTTTTTAGCCCGAGCGAAGATAACTTTACCAACGATCTGAAT
AAAGGCGAAGAAATTACCAGCGATACCAATATTGAAGCAGCCGAAGAAAACATTAGCCTGGATCTGAT
TCAGCAGTATTATCTGACCTTCAACTTTGATAACGAGCCGGAAAATATCAGCATTGAAAATCTGAGCA
GCGATATTATTGGTCAGCTGGAACTGATGCCGAATATTGAACGTTTTCCGAACGGCAAAAAATACGAG
CTGGATAAATACACCATGTTCCATTATCTGCGTGCCCAAGAATTTGAACATGGTAAAAGCCGTATTGC
CCTGACCAATTCAGTTAATGAAGCACTGCTGAACCCGAGCCGTGTTTATACCTTTTTTAGCAGCGATT
ACGTGAAAAAGGTGAACAAAGCAACCGAAGCAGCAATGTTTTTAGGTTGGGTTGAACAGCTGGTGTAT
GATTTCACCGATGAAACCAGCGAAGTTAGCACCACCGATAAAATTGCAGATATCACCATTATCATCCC
GTATATTGGTCCGGCACTGAATATTGGCAATATGCTGTATAAAGATGATTTCGTGGGTGCCCTGATTT
TTAGCGGTGCAGTTATTCTGCTGGAATTTATTCCGGAAATTGCCATTCCGGTTCTGGGCACCTTTGCA
CTGGTTAGCTATATTGCAAATAAAGTTCTGACCGTGCAGACCATTGATAATGCACTGAGCAAACGTAA
CGAGAAATGGGATGAAGTGTACAAATATATCGTGACCAATTGGCTGGCCAAAGTTAATACCCAGATTG
ATCTGATCCGCAAAAAAATGAAAGAAGCCCTGGAAAATCAGGCAGAAGCAACCAAAGCCATTATCAAC
TATCAGTATAACCAGTACACCGAAGAAGAGAAAAACAACATCAACTTCAACATCGATGACCTGAGCAG
CAAACTGAATGAAAGCATCAATAAGGCCATGATTAACATCAACAAATTTCTGAATCAGTGCAGCGTGA
GCTATCTGATGAATAGCATGATTCCGTATGGTGTGAAACGCCTGGAAGATTTTGATGCAAGCCTGAAA
GATGCGCTGCTGAAATATATCTATGATAATCGTGGCACCCTGATTGGCCAGGTTGATCGTCTGAAAGA
TAAAGTTAACAATACCCTGAGTACCGACATTCCGTTTCAGCTGAGCAAATATGTTGATAATCAGCGTC
TGCTGAGCACCTTTACCGAATATATCAAGAACATCATCAACACCAGCATTCTGAATCTGCGCTATGAA
AGCAATCATCTGATCGATCTGAGCCGTTATGCAAGCAAAATCAACATTGGTAGCAAAGTGAACTTCGA
CCCGATTGATAAAAACCAGATTCAGCTGTTTAATCTGGAAAGCAGCAAAATCGAAGTGATCCTGAAAA
ACGCCATTGTGTATAACAGCATGTATGAGAATTTCTCGACCAGCTTTTGGATTCGCATTCCGAAATAC
TTTAATAGCATCAGCCTGAACAACGAGTACACCATTATTAACTGCATGGAAAACAATAGCGGTTGGAA
AGTGAGCCTGAATTATGGTGAAATTATCTGGACCCTGCAGGATACCCAAGAAATCAAACAGCGTGTTG
TGTTCAAATACAGCCAGATGATTAATATCAGCGACTATATCAACCGCTGGATCTTTGTTACCATTACC
AATAATCGCCTGAATAACAGCAAGATCTATATTAACGGTCGCCTGATTGATCAGAAACCGATTAGCAA
TCTGGGCAATATTCATGCGAGCAACAACATTATGTTTAAACTGGATGGTTGCCGTGATACCCATCGTT
ATATTTGGATCAAATACTTCAACCTGTTTGATAAAGAACTGAACGAAAAAGAAATTAAAGACCTGTAC
GACAACCAGAGCAATTCCGGTATTCTGAAAGACTTTTGGGGAGATTATCTGCAGTATGACAAACCGTA
TTATATGCTGAACCTGTATGACCCGAACAAATATGTGGATGTGAACAATGTTGGTATCCGTGGCTATA
TGTATCTGAAAGGTCCGCGTGGTAGCGTTggtACCACCAACATTTATCTGAATAGCAGCCTGTATCGC
GGTACGAAATTTATCATTAAAAAGTATGCCAGCGGCAACAAGGATAATATTGTGCGTAATAATGATCG
CGTGTACATTAACGTTGTGGTGAAGAATAAAGAATATCGCCTGGCAACCAATGCAAGCCAGGCAGGCG
TTGAAAAAATTCTGAGCGCACTGGAAATTCCGGATGTTGGTAATCTGAGCCAGGTTGTTGTTATGAAA
AGCAAAAATGATCAGGGCATCACCAACAAGTGCAAAATGAATCTGCAGGACAATAACGGCAACGATAT
TGGTTTTATTGGCTTCCACCAGTTCAACAATATTGCGAAACTGGTTGCAAGCAATTGGTATAATCGTC
AGATTGAACGTAGCAGTCGTACCCTGGGTTGTAGCTGGGAATTTATCCCTGTGGATGATGGTTGGGGT
GAACGTCCGCTGTAA
SEQ ID NO: 138
(Modified BoNT/A1 (M1144L) Nucleic Acid Sequence)
ATGCCGTTTGTGAACAAACAGTTCAACTATAAAGATCCGGTGAACGGTGTTGATATCGCCTATATCAA
AATTCCGAATGCAGGTCAGATGCAGCCGGTTAAAGCCTTTAAAATCCATAACAAAATTTGGGTGATTC
CGGAACGTGATACCTTTACCAATCCGGAAGAAGGTGATCTGAATCCGCCTCCGGAAGCAAAACAGGTT
CCGGTTAGCTATTATGATAGCACCTATCTGAGCACCGATAACGAGAAAGATAACTATCTGAAAGGTGT
GACCAAACTGTTTGAACGCATTTATAGTACCGATCTGGGTCGTATGCTGCTGACCAGCATTGTTCGTG
GTATTCCGTTTTGGGGTGGTAGCACCATTGATACCGAACTGAAAGTTATTGACACCAACTGCATTAAT
GTGATTCAGCCGGATGGTAGCTATCGTAGCGAAGAACTGAATCTGGTTATTATTGGTCCGAGCGCAGA
TATCATTCAGTTTGAATGTAAATCCTTTGGCCACGAAGTTCTGAATCTGACCCGTAATGGTTATGGTA
GTACCCAGTATATTCGTTTCAGTCCGGATTTTACCTTTGGCTTTGAAGAAAGCCTGGAAGTTGATACA
AATCCGCTGTTAGGTGCAGGTAAATTTGCAACCGATCCGGCAGTTACCCTGGCACATGAACTGATTCA
TGCCGGTCATCGTCTGTATGGTATTGCAATTAATCCGAACCGTGTGTTCAAAGTGAATACCAACGCAT
ATTATGAAATGAGCGGTCTGGAAGTGTCATTTGAAGAACTGCGTACCTTTGGTGGTCATGATGCCAAA
TTTATCGATAGCCTGCAAGAAAATGAATTTCGCCTGTACTACTATAACAAATTCAAGGATATTGCGAG
CACCCTGAATAAAGCCAAAAGCATTGTTGGCACCACCGCAAGCCTGCAGTATATGAAAAATGTGTTTA
AAGAAAAATATCTGCTGAGCGAAGATACCAGCGGTAAATTTAGCGTTGACAAACTGAAATTCGATAAA
CTGTACAAGATGCTGACCGAGATTTATACCGAAGATAACTTCGTGAAGTTTTTCAAAGTGCTGAACCG
CAAAACCTACCTGAACTTTGATAAAGCCGTGTTCAAAATCAACATCGTGCCGAAAGTGAACTATACCA
TCTATGATGGTTTTAACCTGCGCAATACCAATCTGGCAGCAAACTTTAATGGTCAGAACACCGAAATC
AACAACATGAACTTTACCAAACTGAAGAACTTCACCGGTCTGTTCGAATTTTACAAACTGCTGTGTGT
TCGTGGCATTATTACCtCCAAAACCAAAAGCCTGGATAAAGGTTATAACAAAGCCCTGAATGACCTGT
GCATTAAAGTGAATAATTGGGACCTGTTTTTTAGCCCGAGCGAAGATAACTTTACCAACGATCTGAAT
AAAGGCGAAGAAATTACCAGCGATACCAATATTGAAGCAGCCGAAGAAAACATTAGCCTGGATCTGAT
TCAGCAGTATTATCTGACCTTCAACTTTGATAACGAGCCGGAAAATATCAGCATTGAAAATCTGAGCA
GCGATATTATTGGTCAGCTGGAACTGATGCCGAATATTGAACGTTTTCCGAACGGCAAAAAATACGAG
CTGGATAAATACACCATGTTCCATTATCTGCGTGCCCAAGAATTTGAACATGGTAAAAGCCGTATTGC
CCTGACCAATTCAGTTAATGAAGCACTGCTGAACCCGAGCCGTGTTTATACCTTTTTTAGCAGCGATT
ACGTGAAAAAGGTGAACAAAGCAACCGAAGCAGCAATGTTTTTAGGTTGGGTTGAACAGCTGGTGTAT
GATTTCACCGATGAAACCAGCGAAGTTAGCACCACCGATAAAATTGCAGATATCACCATTATCATCCC
GTATATTGGTCCGGCACTGAATATTGGCAATATGCTGTATAAAGATGATTTCGTGGGTGCCCTGATTT
TTAGCGGTGCAGTTATTCTGCTGGAATTTATTCCGGAAATTGCCATTCCGGTTCTGGGCACCTTTGCA
CTGGTTAGCTATATTGCAAATAAAGTTCTGACCGTGCAGACCATTGATAATGCACTGAGCAAACGTAA
CGAGAAATGGGATGAAGTGTACAAATATATCGTGACCAATTGGCTGGCCAAAGTTAATACCCAGATTG
ATCTGATCCGCAAAAAAATGAAAGAAGCCCTGGAAAATCAGGCAGAAGCAACCAAAGCCATTATCAAC
TATCAGTATAACCAGTACACCGAAGAAGAGAAAAACAACATCAACTTCAACATCGATGACCTGAGCAG
CAAACTGAATGAAAGCATCAATAAGGCCATGATTAACATCAACAAATTTCTGAATCAGTGCAGCGTGA
GCTATCTGATGAATAGCATGATTCCGTATGGTGTGAAACGCCTGGAAGATTTTGATGCAAGCCTGAAA
GATGCGCTGCTGAAATATATCTATGATAATCGTGGCACCCTGATTGGCCAGGTTGATCGTCTGAAAGA
TAAAGTTAACAATACCCTGAGTACCGACATTCCGTTTCAGCTGAGCAAATATGTTGATAATCAGCGTC
TGCTGAGCACCTTTACCGAATATATCAAGAACATCATCAACACCAGCATTCTGAATCTGCGCTATGAA
AGCAATCATCTGATCGATCTGAGCCGTTATGCAAGCAAAATCAACATTGGTAGCAAAGTGAACTTCGA
CCCGATTGATAAAAACCAGATTCAGCTGTTTAATCTGGAAAGCAGCAAAATCGAAGTGATCCTGAAAA
ACGCCATTGTGTATAACAGCATGTATGAGAATTTCTCGACCAGCTTTTGGATTCGCATTCCGAAATAC
TTTAATAGCATCAGCCTGAACAACGAGTACACCATTATTAACTGCATGGAAAACAATAGCGGTTGGAA
AGTGAGCCTGAATTATGGTGAAATTATCTGGACCCTGCAGGATACCCAAGAAATCAAACAGCGTGTTG
TGTTCAAATACAGCCAGATGATTAATATCAGCGACTATATCAACCGCTGGATCTTTGTTACCATTACC
AATAATCGCCTGAATAACAGCAAGATCTATATTAACGGTCGCCTGATTGATCAGAAACCGATTAGCAA
TCTGGGCAATATTCATGCGAGCAACAACATTATGTTTAAACTGGATGGTTGCCGTGATACCCATCGTT
ATATTTGGATCAAATACTTCAACCTGTTTGATAAAGAACTGAACGAAAAAGAAATTAAAGACCTGTAC
GACAACCAGAGCAATTCCGGTATTCTGAAAGACTTTTGGGGAGATTATCTGCAGTATGACAAACCGTA
TTATATGCTGAACCTGTATGACCCGAACAAATATGTGGATGTGAACAATGTTGGTATCCGTGGCTATA
TGTATCTGAAAGGTCCGCGTGGTAGCGTTcTGACCACCAACATTTATCTGAATAGCAGCCTGTATCGC
GGTACGAAATTTATCATTAAAAAGTATGCCAGCGGCAACAAGGATAATATTGTGCGTAATAATGATCG
CGTGTACATTAACGTTGTGGTGAAGAATAAAGAATATCGCCTGGCAACCAATGCAAGCCAGGCAGGCG
TTGAAAAAATTCTGAGCGCACTGGAAATTCCGGATGTTGGTAATCTGAGCCAGGTTGTTGTTATGAAA
AGCAAAAATGATCAGGGCATCACCAACAAGTGCAAAATGAATCTGCAGGACAATAACGGCAACGATAT
TGGTTTTATTGGCTTCCACCAGTTCAACAATATTGCGAAACTGGTTGCAAGCAATTGGTATAATCGTC
AGATTGAACGTAGCAGTCGTACCCTGGGTTGTAGCTGGGAATTTATCCCTGTGGATGATGGTTGGGGT
GAACGTCCGCTGTAA
SEQ ID NO: 139
(Polypeptide Sequence of BoNT/A2 - UniProt D3IV23)
MPFVNKQFNYKDPVNGVDIAYIKIPNAGQMQPVKAFKIHNKIWVIPERDTFTNPEEGDLN
PPPEAKQVPVSYYDSTYLSTDNEKDNYLKGVTKLFERIYSTDLGRMLLTSIVRGIPFWGG
STIDTELKVIDTNCINVIQPDGSYRSEELNLVIIGPSADIIQFECKSFGHDVLNLTRNGY
GSTQYIRFSPDFTFGFEESLEVDTNPLLGAGKFATDPAVTLAHELIHAEHRLYGIAINPN
RVFKVNTNAYYEMSGLEVSFEELRTFGGHDAKFIDSLQENEFRLYYYNKFKDVASTLNKA
KSIIGTTASLQYMKNVFKEKYLLSEDTSGKFSVDKLKFDKLYKMLTEIYTEDNFVNFFKV
INRKTYLNFDKAVFRINIVPDENYTIKDGFNLKGANLSTNFNGQNTEINSRNFTRLKNFT
GLFEFYKLLCVRGIIPFKTKSLDEGYNKALNDLCIKVNNWDLFFSPSEDNFTNDLDKVEE
ITADTNIEAAEENISLDLIQQYYLTFDFDNEPENISIENLSSDIIGQLEPMPNIERFPNG
KKYELDKYTMFHYLRAQEFEHGDSRIILTNSAEEALLKPNVAYTFFSSKYVKKINKAVEA
FMFLNWAEELVYDFTDETNEVTTMDKIADITIIVPYIGPALNIGNMLSKGEFVEAIIFTG
VVAMLEFIPEYALPVFGTFAIVSYIANKVLTVQTINNALSKRNEKWDEVYKYTVTNWLAK
VNTQIDLIREKMKKALENQAEATKAIINYQYNQYTEEEKNNINFNIDDLSSKLNESINSA
MININKFLDQCSVSYLMNSMIPYAVKRLKDFDASVRDVLLKYIYDNRGTLILQVDRLKAE
VNNTLSTDIPFQLSKYVENKKLLSTFTEYIKNITNTSILSIVVDKDDELIDLSRYGAEIY
RGDKVFYNSIDKNQIKLINLESSTIEVILKNAIVYNSMYENFSTSFWIKIPKYFSKINLN
NEYTIINCIENNSGWKVSLNYGEIIWTLQDNKQNIQRVVFKYSQMVNISDYINRWIFVTI
TNNRLTKSKIYINGRLIDQKLISNLGNIHASNKIMFKLDGCRDPGRYIVIKYFNLFDKEL
NEKEIKDLYDSQSNSGILKDFWGDYLQYDKPYYMLNLYDPNKYVDVNNIGIRGYMYLKGP
RGSVVTTNIYLNSTLYMGTKFIIKKYASGNEDNIVRNNDRVYINVVVKNKEYRLATNASQ
AGVEKILSALEIPDVGNLSQLVVMKSKDDQGIRNKCKMNLQDNNGNDIGLIGFHQFNNIA
KLVASNWYNRQVGKASRTFGCSWEFIPVDDGWGESSQ
SEQ ID NO: 140
(Polypeptide Sequence of BoNT/A5 v.1 - UniProt C7BEA8)
MPFVNKQFNYKDPVNGVDIAYIKIPNAGQMQPVKAFKIHNKIWVIPERDTFTNPEEGDLN
PPPEAKQVPVSYYDSTYLSTDNEKDNYLKGVTKLFERIYSTELGRMLLTSIVRGIPFWGG
STIDTELKVIDTNCINVIQPDGSYRSEELNLVIIGPSADIIQFECKSFGHDVLNLTRNGY
GSTQYIRFSPDFTFGFEESLEVDTNPLLGAGKFATDPAVTLAHELIHAGHRLYGIAINPN
RVFKVNTNAYYEMSGLEVSFEELRTFGEHDAKFIDSLQENEFRLYYYNKFKDIASTLNKA
KSIVGTTASLQYMKNVFKEKYLLSEDTSGKFSVDKLKFDKLYKMLTEIYTEDNFVKFFKV
LNRKTYLNFDKAVFKINIVPEVNYTIYDGFNLRNTNLAANFNGQNTEINNMNFTKLKNFT
GLFEFYKLLCVRGIITSKTKSLDEGYNKALNDLCIKVNNWDLFFSPSEDNFTNDLNKGEE
ITSDTNIEAAEENISLDLIQQYYLTFNFDNEPENISIENLSSDIIGQLELMPNIERFPNG
KKYELDKYTMFHYLRAQEFEHGKSRIVLTNSVNEALLNPSSVYTFFSSDYVRKVNKATEA
AMFLGWVEQLVYDFTDETSEVSTTDKIADITIIIPYIGPALNIGNMLYKDDFVGALIFSG
AVILLEFIPEIAIPVLGTFALVSYIANKVLTVQTIDNALSKRNEKWGEVYKYIVTNWLAK
VNTQIDLIRKKMKEALENQAEATKAIINYQYNQYTEEEKNNINFNIGDLSSKLNDSINKA
MININKFLNQCSVSYLMNSMIPYGVKRLEDFDASLKDALLKYIYDNRGTLIGQVDRLKDK
VNNTLSTDIPFQLSKYVDNQRLLSTFTEYIKNIINTSILNLRYESNHLIDLSRYASEINI
GSKVNFDPIDKNQIQLFNLESSKIEIILKNAIVYNSMYENFSTSFWIKIPKYFSKINLNN
EYTIINCIENNSGWKVSLNYGEIIWTLQDNKQNIQRVVFKYSQMVAISDYINRWIFITIT
NNRLNNSKIYINGRLIDQKPISNLGNIHASNNIMFKLDGCRDPQRYIWIKYFNLFDKELN
EKEIKDLYDNQSNSGILKDFWGNYLQYDKPYYMLNLYDPNKYVDVNNVGIRGYMYLKGPR
GSIVTTNIYLNSSLYMGTKFIIKKYASGNKDNIVRNNDRVYINVVVKNKEYRLATNASQA
GVEKILSVLEIPDVGNLSQVVVMKSKNDQGIRNKCKMNLQDNNGNDIGFIGFHQFNNIDK
LVASNWYNRQIERSSRTFGCSWEFIPVDDGWGESPL
SEQ ID NO: 141
(Polypeptide Sequence of BoNT/A5 v.2 - UniProt C1IPK2)
MLFVNKQFNYKDPVNGVDIAYIKIPNAGQMQPVKAFKIHNKIWVIPERDTFTNPEEGDLN
PPPEAKQVPVSYYDSTYLSTDNEKDNYLKGVTKLFERIYSTELGRMLLTSIVRGIPFWGG
STIDTELKVIDTNCINVIQPDGSYRSEELNLVIIGPSADIIQFECKSFGHDVLNLTRNGY
GSTQYIRFSPDFTFGFEESLEVDTNPLLGAGKFATDPAVTLAHELIHAGHRLYGIAINPN
RVFKVNTNAYYEMSGLEVSFEELRTFGEHDAKFIDSLQENEFRLYYYNKFKDIASTLNKA
KSIVGTTASLQYMKNVFKEKYLLSEDTSGKFSVDKLKFDKLYKMLTEIYTEDNFVKFFKV
LNRKTYLNFDKAVFKINIVPEVNYTIYDGFNLRNTNLAANFNGQNTEINNMNFTKLKNFT
GLFEFYKLLCVRGIITSKTKSLDEGYNKALNDLCIKVNNWDLFFSPSEDNFTNDLNKGEE
ITSDTNIEAAEENISLDLIQQYYLTFNFDNEPENISIENLSSDIIGQLELMPNIERFPNG
KKYELDKYTMFHYLRAQEFEHGKSRIVLINSVNEALLNPSSVYTFFSSDYVRKVNKATEA
AMFLGWVEQLVYDFTDETSEVSTTDKIADITIIIPYIGPALNIGNMLYKDDFVGALIFSG
AVILLEFIPEIAIPVLGTFALVSYIANKVLTVQTIDNALSKRNEKWGEVYKYIVTNWLAK
VNTQIDLIRKKMKEALENQAEATKAIINYQYNQYTEEEKNNINFNIGDLSSKLNDSINKA
MININKFLNQCSVSYLMNSMIPYGVKRLEDFDASLKDALLKYIYDNRGTLIGQVDRLKDK
VNNTLSTDIPFQLSKYVDNQRLLSTFTEYIKNIINTSILNLRYESNHLIDLSRYASEINI
GSKVNFDPIDKNQIQLFNLESSKIEIILKNAIVYNSMYENFSTSFWIKIPKYFSKINLNN
EYTIINCIENNSGWKVSLNYGEIIWTLQDNKQNIQRVVFKYSQMVAISDYINRWIFITIT
NNRLNNSKIYINGRLIDQKPISNLGNIHASNNIMFKLDGCRDPHRYIWIKYFNLFDKELN
EKEIKDLYDNQSNSGILKDFWGNYLQYDKPYYMLNLYDPNKYVDVNNVGIRGYMYLKGPR
GSIVTTNIYLNSSLYMGTKFIIKKYASGNKDNIVRNNDRVYINVVVKNKEYRLATNASQA
GVEKILSVLEIPDVGNLSQVVVMKSKNDQGIRNKCKMNLQDNNGNDIGFIGFHQFNNIDK
LVASNWYNRQIERSSRTFGCSWEFIPVDDGWGESPL
SEQ ID NO: 142
(Polypeptide Sequence of BoNT/A6 - ACW83608.1, Accession # FJ981696)
MPFVNKQFNYKDPVNGVDIAYIKIPNAGQMQPVKAFKIHNKIWVIPERDTFTNPEEGDLNPPPEAKQVPV
SYYDSTYLSTDNEKDNYLKGVTKLFERIYSTDLGRMLLTSIVRGIPFWGGSTIDTELKVIDTNCINVIQP
DGSYRSEELNLVIIGPSADIIQFECKSFGHEVLNLTRNGYGSTQYIRFSPDFTFGFEESLEVDTNPLLGA
GKFATDPAVTLAHELIHAGHRLYGIAINPNRVFKVNTNAYYEMSGLEVSFEELRTFGGHDAKFIDSLQEN
EFRLYYYNKFKDIASTLNKAKSIVGTTASLQYMKNVFKEKYLLSEDTSGKFSVDKLKFDKLYKMLTEIYT
EDNFVKFFKVLNRKTYLNFDKAVFKINIVPKVNYTIYDGFNLRNTNLAANFNGQNTEINNMNFAKLKNFT
GLFEFYKLLCVRGIITSKTKSLDKGYNKALNDLCIKVNNWDLFFSPSEDNFTNDLNKGEEITSDTNIEAA
EENISLDLIQQYYLTFNFDNEPENISIENLSSDIIGQLELMPNIERFPNGKKYELDKYTMFHYLSAQEFE
HGKSRIDLTNSVNEALLNPSHVYTFFSSDYVKKVNKATEAAMFLGWVEQLVYDFTDETSEVSTTDKIADI
TIIIPYIGPALNIGNMLYKDDFVGALIFSGAVILLEFIPEIAIPVLGTFAIVSYIANKVLTVQTINNALS
KRNEKWDEVYKYTVTNWLAKVNTQIDLIREKMKKALENQAEATKAIINYQYNQYTEEEKNNINFNIDDLS
SKLNESINSAMININKFLDQCSVSYLMNSMIPYAVKRLKDFDASVRDVLLKYIYDNRGTLIGQVDRLKDK
VNNTLSTDIPFQLSKYVDNQRLLSTFTEYIKNIINTSILSLRYENNHLIDLSRYASKINIGSRVNFDPID
KNQIQLFNLESSKIEVILKNAIVYNSMYENFSTSFWIKIPKYFSEISLNNEYTIINCIENNSGWKVSLNY
GEIIWTLQDNKQNIQRVVFKYSQMVAISDYINRWIFITITNNRLTKSKIYINGRLIDQKPISNLGNIHAS
NKIMFKLDGCRDPRRYIMIKYFNLFDKELNEKEIKDLYDSQSNSGILKDFWGNYLQYDKPYYMLNLFDPN
KYVDVNNVGIRGYMYLKGSRSTLLTTNIYLNSGLYMGTKFIIKKYASGNKDNIVRNNDRVYINVVVNNKE
YRLATNASQAGVEKILSALEIPDIGNLSQVVVMKSKNDQGIRNKCKMNLQDNNGNDIGFIGFHKFNDIYK
LVASNWYNRQIEISSRTFGCSWEFIPVDDGWGEKPL
SEQ ID NO: 143
(Polypeptide Sequence of BoNT/A7 - GenBank: AFV13854.1, Accession#
JQ954969.1)
MPFVNKQFNYKDPVNGVDIAYIKIPNAGQMQPVKAFKIHNKIWVIPERDIFTNPEEGDLNPPPEAKQVPV
SYYDSTYLSTDNEKDNYLKGVTKLFERIYSTDLGRMLLTSIVRGIPFWGGSTIDTELKVIDTNCINVIQP
DGSYRSEELNLVIIGPSADIINFECKSFGHDVLNLTRNGYGSTQYIRFSPDFTFGFEESLEVDTNPLLGA
GKFAIDPAVTLAHELIHAGHRLYGIAINPNRVFKVNTNAYYEMSGLEVSFEELRTFGGHDAKFIDSLQEN
EFRLYYYNKFKEVASILNKAKSIIGTTASLQYMKNVFKEKYLLSEDTSGKFSVDKLRFDKLYKMLTEIYT
EDNFVKFFKVLNRKTYLNFDKAVFKMNIVPEVNYTIYDGFNLRNTNLAANFNGQNTEINNMNFTKLKNFT
GLFEFYKLLCVRGIITSKTKSLDEGYNKALNDLCIKVNNWDLFFSPSEDNFTNDLNKGEEITSDTNIEAA
EENISSDLIQQYYLTFNFDNEPENISIENLSSDIIGQLELMPNIERFPNGKKYELDKYTMFHYLRAQEFE
YGNSRIVLINSVNEALLNPSSVYTFFSSDYVKKANEATEAAMFLGWVEQLVYDFTDETSEVSTMDKIADI
TIIVPYIGPALNIGNMVYKKKFEEALIFSGAVILLEFVPEIVLPILGTFALVSYTSNKVLTVRTIDNALS
KRNEKWEEVYKYIVTNWLAKVNTQINLIRKKMKEALENQAEATKAIINYQYNQYTEEEKNNINFNIGDLS
SKLNDSINKAMININKFLDQCSVSYLMNSMIPQGVKQLKDFDTSLRDSLLKYIYDNRGTLIGQVDRLKDK
VNNTLSTDIPFQLSKYADNQRLLSTFTEYIKNIINTSILNLRYESNHLIDLSRYASKINIGSRVNFDPID
KNQIQLFNLESSKIEVILKNAIVYNSMYENFSTSFWIKIPKYFSKINLNNEYTIINCIENNSGWKVSLNY
GEIIWTLQDNEQNIQRVVFKYSQMVNISDYINRWIFVTITNNRLTKSKIYINGRLIDQKPISNLGNIHAS
NKIMFKLDGCRDPHRYILIKYFNLFDKELNEKEIKDLYDNQSNSGILKDFWGDYLQYDKPYYMLNLYDPN
KYIDVNNIGIRGYMYLKGPRGSVTTTNIYLNSMLYMGTKFIIKKHASGNKDNIVRNNDRVYINVLVKNKE
YRLATNASQAGGEKILSAVEIPDVGNLSQVVVMKSKNDQGIRNKCKMNLQDNNGNDIGFIGFHQFNNIAK
LVASNWYNRQIGKTSVTLGCSWELIPVDYGWGESSL
SEQ ID NO: 144
(Polypeptide Sequence of BoNT/A8 - GenBank: AJA05787.1, Accession#
KM233166)
MPFVNKQFNYKDTVNGIDIAYIKIPNAGQMQPVKAFKIHNKIWVIPERDTFTNPKEGDLNPPPEAKQVPV
SYYDSTYLSTDNEKDNYLKGVTKLFERIYSTDLGRMLLTSIVRGIPFWGGSTIDTELKVIDTNCINVIQP
DGSYRSEELNLVIIGPSADIIQFECKSFGHDVLNLTRNGYGSTQYIRFSPDFTFGFEESLEVDTNPLLGA
GKFATDPAVTLAHELIHAEHRLYGIAINPNRVFKVNTNAYYEMSGLEVSFEELRTFGGHNAKFIDSLQEN
EFRLYYYNKFKDIASTLNKAKSIVGTTASLQYMKNVFKEKYLLSEDTSGKFSVDKLKFDKLYKMLTEIYT
EDNFVKFFKVLNRKTYLNFDKAVFKINIVPDENYTIKDGFNLKNTNLAANFNGQNTEINSRNFTKLKNFT
GLFEFYKLLCVRGIIPFKTKSLDEGYNKALNDLCIKVNNWDLFFSPSEDNFTNDLDKVEEITSDTNIEAA
EENISLDLIQQYYLTFDFDNEPENISIENLSSDIIGQLEPMPNIERFPNGKKYELDKYTMFHYLRAQEFE
HSKSRIALTNSVNEALLNPSRVYTFFSSDYVKKVNKATEAAMFLGWVEQLVYDFTDETSEVSTTDKIADI
TIIIPYIGPALNIGNMLYKDDFVGALIFSGAVILLEFIPEIAIPVLGTFALVSYIANKVLTVQTIDNALS
KRNEKWDEVYKYIVTNWLAKVNTQIDLVRKKMKEALENQAEATKAIINYQYNQYTEEEKNNINFNIDDLS
SKLNESINSAMTNINKFLDQCSVSYLMNSMIPYAVKRLKDFDASVREVLLKYIYDNRGTLILQVDRLKDK
VNNTLSADIPFQLSKYVDNKKLLSTFTEYIKNITNTSILSIVVDKDGRLIDLSRYGAEIYNGDKVSYNSI
DKNQIKLINLESSAIEVILKNAIVYNSMYENFSTSFWIKIPKYFSKINLNNEYTIINCIENNSGWKVSLN
YGEIIWTLQDNQQNIQRVVFKYSQMVNISDYINRWIFVTITNNRLDKSKIYINGRLIDQKPISNLGNIHA
SNNIMFKLDGCRDPRRYIVIKYFNLFDKELNEKEIKDLYDNQSNSGILKDFWGDYLQYDKPYYMLNLYDP
NKYVDVNNIGIRGYMYLKGPRGSVVTTNIYLNSTLYMGTKFIIKKYASGNKDNIVRNNDRVYINVVVKNK
EYRLATNALQAGVEKILSALEIPDVGNLSQVVVMKSKNDQGIRNKCKMNLQDNNGNDIGLIGFHQFNNIA
KLVASNWYNRQVGKASRTFGCSWEFIPVDDGWGESSQ

### EXAMPLES

### Materials & Methods

### Forced Oxidation Study

BoNT molecules were subjected to forced oxidation by exchanging the buffering solution using 0.5 mL Amicon spin columns. The buffer used to oxidise was 0.001% Hydrogen Peroxide in 20 mM Citrate buffer pH 5.0. Following buffer exchange samples were incubated in the dark for the desired period up to 72 hours before the oxidation reaction was quenched by the addition of excess methionine. LC MS peptide mapping was used to calculate oxidation of the molecules tested. Samples were denatured by the addition of 1M Urea in 100 mM Tris pH 7.6 before being reduced and alkylated by the addition of DTT and Iodoacetamide respectively, they were then incubated in the dark for 30 minutes. Samples were then buffer exchanged into 1M Urea in 100 mM Tris pH 7.6 using 0.5 mL Amicon spin columns for digestion. Trypsin was added and the samples were incubated at 37° C. for 4 hours to digest the protein. LC MS was carried out on a Waters SYNAPT G2-Si (Tof) mass spectrometer coupled to an acquity H-Class Bio UPLC. Aliquots of 10 microliters were injected onto an Acquity BEH C18 column 130 Å, 1.7 μm, 2.1×150 mm and analysed by a 93 minute UPLC gradient at a flow rate of 0.2 ml/min. The UPLC gradient is shown in the table below, mobile phase A was composed of 0.02% TFA in water and mobile phase B was composed of 0.02% TFA in acetonitrile.

| Time | % A | % B |
|---|---|---|
| Initial | 100 | 0 |
| 2 | 100 | 0 |
| 5 | 95 | 5 |
| 29 | 80 | 20 |
| 34.5 | 79 | 21 |
| 38.5 | 76 | 24 |
| 73.5 | 65 | 35 |
| 77.5 | 10 | 90 |
| 78.5 | 5 | 95 |
| 80 | 95 | 5 |
| 82 | 5 | 95 |
| 83 | 100 | 0 |
| 93 | 100 | 0 |

Data were acquired using an MS^{E} method comprised of a low energy and a high collision energy scan run on UNIFI software.

### Cell-Based Activity Assay

The cell-based activity assay uses an engineered neuronal rodent clonal cell line. Addition of toxins leads to internalisation via receptor binding, translocation and proteolytic cleavage of the toxin target. The cell line expresses full-length SNAP-25 (natural proteolytic target of BoNT/A), the cleavage of which is measured in the assay. The potency of toxin samples was determined relative to a Reference Standard by a parallel curve test, and by comparing the EC₅₀ of Test Samples with the EC₅₀ of the Reference Standard. On day one cells were dispensed into inner wells of 3 tissue culture plates and allowed to settle for 15-25 minutes then incubated at 37° C. with 5% CO₂ for 1 to 1.5 hours. Media was then changed from growth media to assay media and cells were incubated overnight at 37° C. with 5% CO₂. On day two, samples for investigation were diluted in assay media and added to wells at the desired concentration, the plates were then incubated at 37° C. with 5% CO₂ for 72 hours. On day five the fluorescence was measured using the Tecan INFINITE M1000 PRO plate reader.

### Computer Modelling

Computational analysis to prioritise M1144 substitutions was performed on the BoNT/A crystal structure (3BTA.pdb) using Molecular Operating Environment visualisation software (MOE—Chemical Computing Group ULC). Average structure properties for mutants were evaluated from ensembles generated by the residue scan module. Variation to surface area and hydrophilic/hydrophobic patches and the predicted impact on the 3D confirmation and intramolecular interactions compared to the wild type molecule were assessed. Mutations were prioritised that minimised disruption to the protein structure and the interaction network of the wild-type M1144.

### Mutation of BoNT/A

Mutation of the BoNT/A gene sequence (codon optimised for expression in E. coli) was performed by site directed mutagenesis of the M1144 codon in the expression vector using the Q5 kit from NEB (E0554S). Primers were designed to change the ATG (Methionine) codon to GTG (Valine) or CTG (Leucine) using the NEBase Changer software. The plasmid was amplified by PCR using the resulting primers. The resulting DNA was then treated with a Kinase/Ligase/DpnI mixture to ligate the amplified DNA and digest the plasmid DNA template. The reaction mixture was then mixed with chemically competent E. coli and transformed using heat shock at 42° C. Transformants were selected using antibiotic selection on LB agar plates. Colonies were picked to inoculate overnight cultures. The cultures were harvested by centrifugation and plasmid DNA was prepared for sequencing analysis using a Wizard Plus SV Miniprep Kit (Promega). Mutations were confirmed by Sanger Sequencing. All plasmids used contained a promoter region for the gene of interest under the control of the lac operator and as such expression was inducible by the addition of IPTG.

### Expression, Activation, and Purification of Modified BoNT/A

Expression was initiated with a 100 mL culture inoculated from the selected cell bank and grown overnight at 37° C. in the presence of the selection antibiotic using modified terrific broth. The overnight culture was then used to inoculate the 1 L main culture that was then incubated at 37° C. until an OD_{600 nm} of 0.5-1.0 AU. When the desired OD_{600 nm} was reached the temperature was dropped to 16° C. for 1 hr. Following the temperature drop expression was induced using 1 mM IPTG and the culture was grown for 20 hrs before harvesting by centrifugation.

Cells were resuspended in 3 mL/g wet cell weight in 50 mM Tris pH 8.0, 200 mM NaCl and lysed by sonication (Misonix 3000 sonicator) on ice. The sample was conditioned to load the capture column by the addition of ammonium sulphate (NH₄)₂SO₄ and was then clarified by centrifugation prior to the chromatography step. Target protein was captured using Hydrophobic Interaction Chromatography (HIC) and was eluted over an ammonium sulphate gradient (1M-0M (NH₄)₂SO₄). Fractions were analysed by SDS-PAGE and those containing BoNT were pooled and conditioned for intermediate purification using Anion Exchange Chromatography (AIEX). The conditioned sample was loaded to the Ion exchange column and eluted over an increasing sodium chloride (NaCl) gradient from 25 mM to 1 M. Fractions containing BoNT were pooled following SDS-PAGE analysis and activated using Lys-C. 0.5 pg Lys-C per mg of total protein was added and activation was performed at 2-8° C. for 18 hours. The sample was then conditioned by the addition of NaCl before a final polishing chromatography step using HIC. Protein was eluted over a decreasing NaCl gradient (3 M-0M). Fractions containing activated BoNT were pooled and concentrated to 0.5-1.0 mg/mL and buffer exchanged by size exclusion chromatography into PBS pH 7.2 prior to storage at −80° C.

### Example 1

### Oxidation of Botulinum Neurotoxin A (BoNT/A)

Botulinum neurotoxin A (BoNT/A) was subjected to forced oxidation via exposure to the oxidising agent hydrogen peroxide, as described above. Mass spectrometry was then utilized to assess the level of oxidation of peptides within the BoNT/A molecule and specific sites of oxidation identified/assessed due to a change in mass. Based on the analysis, a number of oxidised BoNT/A peptides were identified.

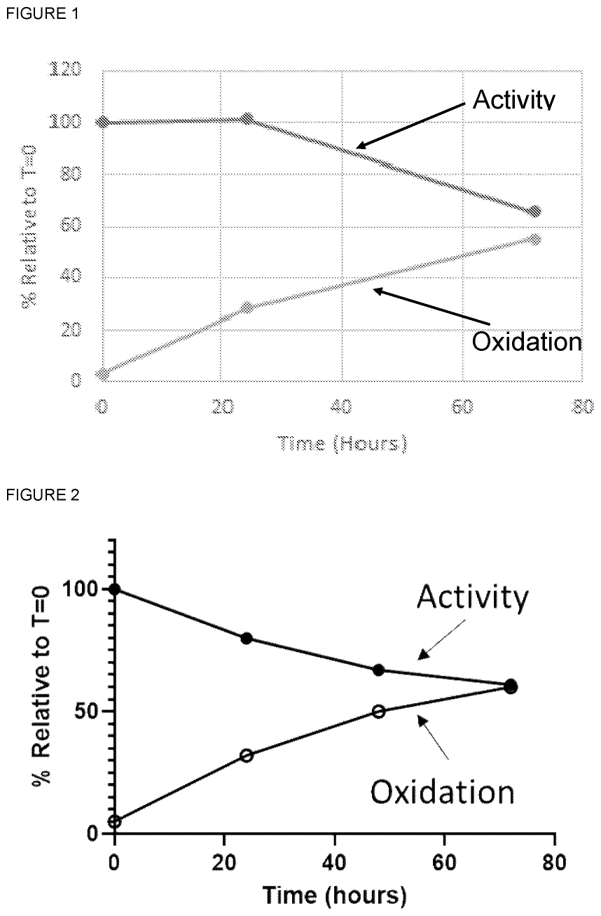

The oxidised BoNT/A was also tested in a cell-based activity assay as described above. It was found that there was a correlation between the number of peptides oxidised at a methionine residue corresponding to position 1144 (M1144) of BoNT/A (FIG. 1 and FIG. 2, “Oxidation”) and activity (FIG. 1 and FIG. 2, “Activity”). In other words, oxidation at M1144 was found to be a principal cause of oxidation-dependent loss of BoNT/A activity.

M1144 is an amino acid present in the C-terminal portion of the He domain of BoNT/A (Hec domain) at a SV2c target binding region.

### Example 2

### Modelling Modification of BoNT/A

Having identified M1144 as a candidate for oxidative sensitivity, a mutation was introduced at this site in an attempt to prevent oxidation at this residue. Computer modelling was performed using an existing BoNT/A crystal structure to predict and score the structural effect of various modifications.

Based on the modelling, the following substitutions were considered: M1144V (valine), M1114G (glycine), M1144L (leucine), M1144T (threonine), M1144A (alanine), and M11441 (isoleucine). M1144V, M1144G, and M1144L were short-listed owing to their lower energy value and reduced impact on hydrophobic and hydrophilic surfaces (versus M1144T), due to an absence of a large side chain, thereby guaranteeing less disturbance to neighbouring side chains (versus M1144A), and due to side chain position being less disruptive (versus M11441), respectively.

### Example 3

Substitution of M1144 with Valine

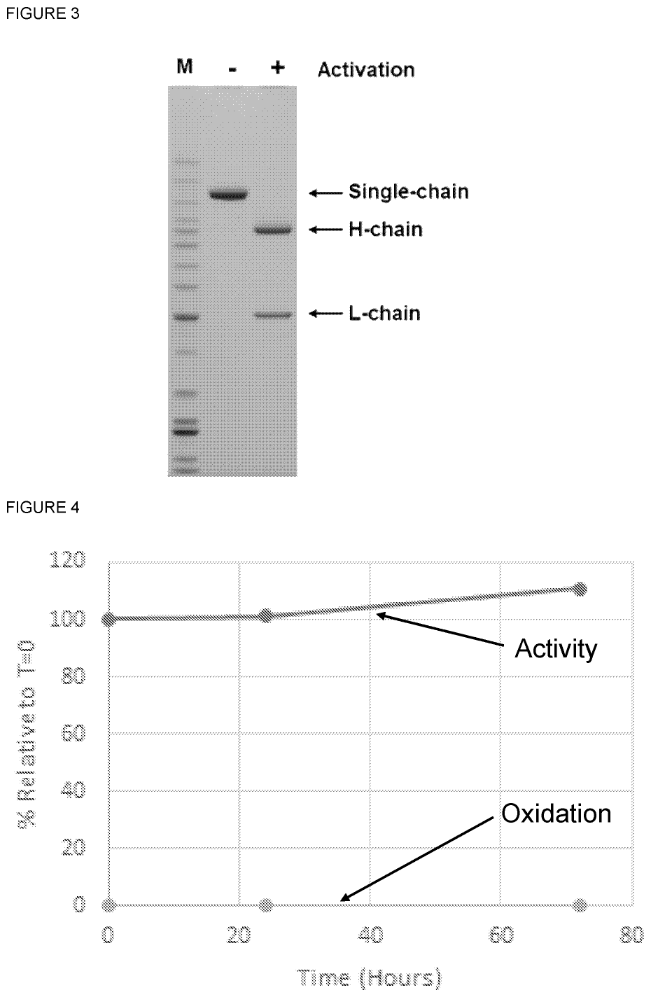

Mutation of M1144 to valine was carried out as described above to produce a modified BoNT/A. The single-chain modified BoNT/A was expressed in E. coli and purified using column chromatography prior to proteolytic activation using Lys-C. FIG. 3 shows that the single-chain modified BoNT/A was of a high level of purity (˜100%, FIG. 3 “−”) and that Lys-C cleaved BoNT/A into the corresponding di-chain form (activation was ˜100%, FIG. 3 “+”).

The modified di-chain BoNT/A was subjected to forced oxidation (as described above) and activity was assessed via the cell-based assay. FIG. 4 shows that the M1144V substitution prevented oxidation at position 1144 (see “Oxidation”). However, surprisingly, the modification not only prevented oxidation-dependent loss of BoNT/A activity, but significantly increased activity of the modified BoNT/A (see FIG. 4, “Activity”).

### Example 4

Substitution of M1144 with Leucine

Mutation of M1144 to leucine was carried out as described above to produce a modified BoNT/A. The single-chain modified BoNT/A was expressed, purified, and activated as described.

### Example 5

### Activity of BoNT/A M1144V and M1144L

BoNT/A M1144L and M1144V were compared with unmodified BoNT/A in a cell-based activity assay. EC₅₀ (half maximal effective concentration) values were determined and the relative activities calculated as follows:

$$( Unmodified BoNT / A {EC}_{50} / Modified BoNT / A {EC}_{50} ) \times 100 = relative activity$$

The activity of unmodified BoNT/A was set at 100%.

TABLE 1
Relative activity of BoNT/A M1144V,
M1144L, and unmodified BoNT/A.

| Molecule | Relative Activity |
|---|---|
| Unmodified | 100% |
| BoNT/A | |
| BoNT/A M1144V | 317% |
| BoNT/A M1144L | 225% |

The table above shows that both BoNT/A M1144V and M1144L were substantially more active than unmodified BoNT/A even under non-oxidising (or low oxidising) conditions.

### Example 6

### Binding of BoNT/A M1144L and BoNT/A M1144V to SV2c

Binding experiments were performed on a Biacore 8K (Cytiva) at 25° C. Immobilisation was performed using HBS-EP+ (10 mM Hepes, 150 mM NaCl, 3 mM EDTA and 0.05% v/v Tween 20, pH 7.4) as the running buffer. Using standard EDC/NHS amine coupling, GST-SV2c was coupled to Fc2 of a series S CM5 sensor chip (Cytiva) to a density of ˜50 Resonance Units (RU). Fc1 was blank immobilised by EDC/NHS before blocking with 1M ethanolamine (Cytiva).

For kinetics measurements, HBS-EP+ supplemented with 0.1 mg/ml BSA was used as the running buffer at a flow rate of 30 μl/min. Botulinum toxins were injected in a 1:1 dilution series comprising 500 to 7.8 nM using single cycle methodology. Association was monitored over 7 increasing concentrations for 200 seconds followed by a single 600 second dissociation. Following each cycle the surface was regenerated with a 60 second injection of 10 mM glycine pH 1.5 (Cytiva) at 10 μl/min.

Binding affinity and kinetics were determined by fitting the double referenced binding curves using the heterogenous binding model (Biacore Insight Evaluation software).

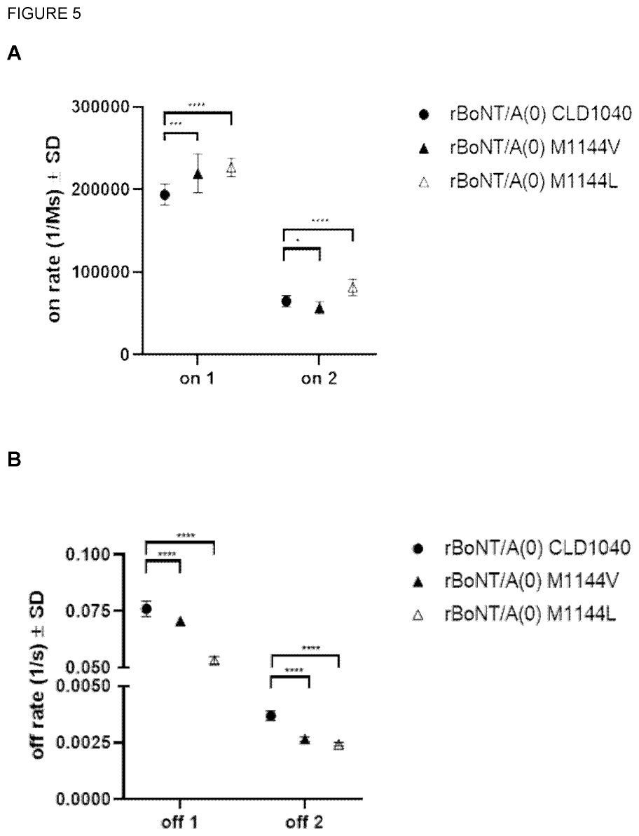

The binding affinity and kinetics of BoNT/A M1144L and BoNT/A M1144V were compared to wild-type BoNT/A expressed in CLD1040 (“BoNT/A CLD1040”). Results are presented in the tables below, as well as graphically in FIGS. 5A and B.

TABLE 2
Association and dissociation rates (ka and kd, respectively) and
dissociation constant (K_{D}) for heterogenous binding model K_{D}1.

| | ka1 | kd1 | K_{D}1 |
|---|---|---|---|
| Molecule | (1/Ms ± SD) | (1/s ± SD) | (nM ± SD) |
| BoNT/A | 1.94E+5 ± | 7.60E−2 ± | 393.5 ± |
| (CLD1040) | 1.27E+4 | 3.44E−3 | 13.1 |
| BoNT/A M1144V | 2.19E+5 ± | 7.06E−2 ± | 324.0 ± |
| | 2.36E+4 | 1.66E−3 | 19.2 |
| BoNT/A M1144L | 2.27E+5 ± | 5.33E−2 ± | 234.8 ± |
| | 1.10E+4 | 1.63E−3 | 6.5 |

TABLE 3
Association and dissociation rates (ka and kd, respectively) and
dissociation constant (K_{D}) for heterogenous binding model K_{D}2.

| | ka2 | kd2 | K_{D}2 |
|---|---|---|---|
| Molecule | (1/Ms ± SD) | (1/s ± SD) | (nM + SD) |
| BoNT/A | 6.49E+4 ± | 3.68E−3 ± | 57.4 ± |
| (CLD1040) | 6.83E+3 | 2.17E−4 | 3.4 |
| BoNT/A M1144V | 5.70E+4 ± | 2.65E−3 ± | 47.4 ± |
| | 7.05E+3 | 1.06E−4 | 5.4 |
| BoNT/A M1144L | 8.18E+4 ± | 2.42E−3 ± | 30.1 ± |
| | 9.98E+3 | 7.92E−5 | 3.3 |

Compared to wild-type BoNT/A, BoNT/A M1144V exhibited a statistically-significant increase in affinity for SV2c in both K_{D}1 and K_{D}2. BoNT/A M144L exhibited an even greater (statically-significant) increase in affinity for SV2c in both K_{D}1 and K_{D}2.

In conclusion, said increased affinity is likely the underlying mechanism of action for the improved activity of BoNT/A M1144V and M1144L.

### Example 7

Substitution of M1144 with Glycine

Mutation of M1144 to glycine was carried out as described above for substitution of M1144 with valine to produce a modified BoNT/A. The single-chain modified BoNT/A was expressed, purified, and activated as described.

### Example 8

### Activity of BoNT/A M1144L and BoNT/A M1144G in Oxidising and Non-Oxidising Conditions

Preparations of BoNT/A M1144L and BoNT/A M1144G were subjected to forced oxidation as described above and their activities tested in a cell-based assay. There was no statistically-significant difference in activity of BoNT/A M1144L or BoNT/A M1144G after 72 hours in oxidising conditions compared to BoNT/A M1144L or BoNT/A M1144G (respectively) prior to exposure to oxidising conditions.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. A modified clostridial neurotoxin comprising a botulinum neurotoxin A (BoNT/A) heavy chain C-terminal (H_{cc}) domain, wherein the H_{cc} domain comprises an amino acid modification at the methionine at a position corresponding to position 1144 of SEQ ID NO: 2 (M1144), wherein the amino acid modification results in increased oxidative resistance of the modified clostridial neurotoxin as compared to an otherwise identical clostridial neurotoxin lacking the modification.

2. A method for producing a modified clostridial neurotoxin, the method comprising expressing a nucleic acid encoding the modified clostridial neurotoxin of claim 1 in a suitable host cell, thereby producing the modified clostridial neurotoxin.

3. (canceled)

4. The modified clostridial neurotoxin of claim 1, wherein the modification is a substitution of M1144 with an amino acid resistant to oxidation.

5. The modified clostridial neurotoxin of claim 1, wherein the modified H_{cc} domain comprises the amino acid sequence of RX₁×₂VX₃TTNIYLNSX₄LYX₅GT (SEQ ID NO: 102), wherein:
X₁ is D or G;
X₂ is S or N;
X₃ is an amino acid that is resistant to oxidation;
X₄ is S or T;
and X₅ is M or R.

6. (canceled)

7. The modified clostridial neurotoxin of claim 1, wherein M1144 is substituted with valine, glycine, leucine, threonine, or isoleucine.

8. The modified clostridial neurotoxin of claim 1, wherein the modification is a deletion of M1144.

9. The modified clostridial neurotoxin of claim 1, wherein the modified H_{cc} domain comprises the amino acid sequence of RX₁×₂VTTNIYLNSX₃LYX₄GT (SEQ ID NO: 111), wherein:
X₁ is D or G;
X₂ is S or N;
X₃ is S or T; and
X₄ is M or R.

10. (canceled)

11. The modified clostridial neurotoxin of claim 1, wherein the BoNT/A H_{cc} domain is a BoNT/A1 H_{cc} domain, a BoNT/A3 H_{cc} domain, or a BoNT/A4 H_{cc} domain.

12-13. (canceled)

14. The modified clostridial neurotoxin of claim 1, comprising an amino acid sequence having at least 90% sequence identity to any one of SEQ ID NOs: 63-69, 71-77, 79-85, 87-93, and 95-101.

15. The modified clostridial neurotoxin of claim 1, wherein the modified clostridial neurotoxin is a modified BoNT/A1, a modified BoNT/A3, or a modified BoNT/A4.

16. The modified clostridial neurotoxin of claim 1, wherein the modified clostridial neurotoxin is a modified BoNT/A1 further comprising modification of one or more of:
the asparagine at position 886, 905, 918, 930, 954, 1006, 1025, 1026, 1032, 1043, 1046, 1052, 1080, 1188, 1216, 1242, or 1243;
the glutamine at position 915, 991, 995, or 1229;
the glutamic acid at position 920, 992, 1081, or 1083;
the serine at position 995 or 1274;
the aspartic acid at position 1058, 1086, or 1213;
the histidine at position 1064; the glycine at position 1215; or
the threonine at position 1277;
wherein the residue positions correspond to positions in SEQ ID NO: 2.

17. (canceled)

18. The modified clostridial neurotoxin of claim 1, comprising an amino acid sequence having at least 90% sequence identity to any one of SEQ ID NOs: 3-9, 12-18, 21-27, 30-36, 39-45, 47-53, and 55-61.

19. The modified clostridial neurotoxin of claim 1, wherein the modified clostridial neurotoxin is a single-chain modified clostridial neurotoxin.

20. The modified clostridial neurotoxin of claim 1, wherein the modified clostridial neurotoxin is a di-chain modified clostridial neurotoxin comprising a light chain and a heavy chain joined together by a disulphide bond.

21-25. (canceled)

26. A nucleic acid encoding the modified clostridial neurotoxin of claim 1.

27. (canceled)

28. A method of activating a modified clostridial neurotoxin, the method comprising contacting the single-chain modified clostridial neurotoxin of claim 19 with a protease that cleaves the single-chain modified clostridial neurotoxin at a cleavage site located between the light chain and heavy chain, thereby converting the single-chain modified clostridial neurotoxin into a di-chain modified clostridial neurotoxin, wherein the light chain and heavy chain are joined together by a disulphide bond.

29. A di-chain modified clostridial neurotoxin obtainable by the method of claim 28.

30. A pharmaceutical composition comprising the modified clostridial neurotoxin of claim 1 and a pharmaceutically acceptable carrier, excipient, adjuvant, propellant and/or salt.

31-32. (canceled)

33. A method of treating a disorder, the method comprising administering the modified clostridial neurotoxin of claim 1 to a subject, wherein the disorder is selected from: a condition associated with unwanted immune secretion, strabismus, blepharospasm, squint, dystonia, torticollis, a neuromuscular disorder or condition of ocular motility, a cosmetic disorder, writer's cramp, bruxism, Wilson's disease, tremor, tics, segmental myoclonus, spasms, spasticity due to chronic multiple sclerosis, spasticity resulting in abnormal bladder control, animus, back spasm, charley horse, tension headaches, levator pelvic syndrome, spina bifida, tardive dyskinesia, Parkinson's disease, stuttering, hemifacial spasm, eyelid disorder, cerebral palsy, focal spasticity, spasmodic colitis, neurogenic bladder, anismus, limb spasticity, anal fissure, achalasia, dysphagia, lacrimation, hyperhydrosis, excessive salivation, excessive gastrointestinal secretions, muscle pain, headache pain, cancer, uterine disorders, uro-genital disorders, urogenital-neurological disorders, chronic neurogenic inflammation, and a smooth muscle disorder.

34. (canceled)

35. A method of cosmetic treatment, the method comprising administering the modified clostridial neurotoxin of claim 1, to a subject.

36-37. (canceled)
